(19) United States
(12) Patent Application Publication
(10) Pub. No.: US 2009/0169582 A1
(43) Pub. Date: Jul. 2, 2009

(54) RECOMBINANT LACTOBACILLUS AND USE OF THE SAME

(75) Inventors: Kaw Yan Chua, Singapore (SG); Lay Hong Renee Lim, Singapore (SG); Li Kiang Tan, Singapore (SG)

Correspondence Address: KENYON & KENYON LLP, ONE BROADWAY, NEW YORK, NY, 10004, US

(73) Assignee: NATIONAL UNIVERSITY OF SINGAPORE, Singapore (SG)

(21) Appl. No.: 12/297,691

(22) PCT Filed: Apr. 4, 2007
(86) PCT No.: PCT/SG2007/000090

§ 371 Date: Jan. 20, 2009

Related U.S. Application Data
(60) Provisional application No. 60/793,706, filed on Apr. 20, 2006.

(51) Int. Cl. A61K 39/07; C12N 1/21; A61P 37/08; A61P 37/00

(52) U.S. Cl. 424/200.1; 435/252.31

(57) ABSTRACT

The invention relates to recombinant lactobacillus, use of the same and a pharmaceutical composition including the same. The recombinant lactobacillus includes a heterologous nucleic acid sequence. The heterologous nucleic acid sequence encodes at least an immunogenic fragment of the mite allergens Der p 1, Der p 2, or Blo t 5, or an immunogenic homolog thereof. A respective fragment of Der p 1 includes at least 8% of the amino acid sequence of the mite allergen. A method of modulating the immune response to an allergen in a mammal as well as a pharmaceutical composition and a kit are also disclosed.

### FIELD OF THE INVENTION

The present invention relates to a recombinant lactobacillus and use of the same. The recombinant lactobacillus includes a heterologous nucleic acid sequence encoding at least an immunogenic fragment of a mite allergen, or an immunogenic homolog thereof. The present invention also relates to a method of modulating the immune response to an allergen in a mammal as well as to pharmaceutical compositions and kits.

### BACKGROUND OF THE INVENTION

Allergic diseases are thought to affect between 25 to 40 percent of the population in developed countries, with more than half the US population being sensitized to one or more allergens. Allergy rates are rapidly increasing, especially among children, at a rate of approximately five percent per year in the UK. Allergic reactions such as allergic rhinitis (hay fever), asthma, and hives (urticaria) are an oversensitivity of the immune system response, i.e. a pathological response of the immune system to a respective allergen. In a susceptible person, a normally harmless substance such as grass pollen or house dust, leads to an overreaction of the immune response, so that the respective substance, the allergen, is perceived as a threat and is attacked. Sensitivity to allergens of mites, in particular a house dust mite, is one of the most important factors contributing to the development of allergic asthma, rhinitis and atopic dermatitis. An allergic reaction to a mite allergen may also cause symptoms of hay fever, such as sneezing, runny nose and itchy, watery eyes. Mite allergy constitutes a complex worldwide problem, with sanitary and economical implications. While at least 45% of young people in the US and 15% of the general population of Germany are allergic to dust mite allergens, mite allergy is not restricted to the human “indoor” environment. Many more mite species have been found that can induce sensitization, the symptoms of which are encountered in occupational settings.

An allergic disease is characterized by an increased ability of B-lymphocytes to produce an antibody type known as immunoglobulin E (IgE). Evidence suggests that IgE plays for instance a major role in asthma. For example children or adults who respond in a modified form, in which levels of IgG and IgG4 immunoglobulins but not IgE are increased, are not at increased risk of asthma. The synthesis of IgE is a result of collaboration between subsets of T helper cells, CD4⁺ and B cells. A pivotal role in e.g. atopic allergy is played by T cells, and cytokines synthesized by type 2 helper T cells (Th2) contribute significantly to the disease pathogenesis. It has been postulated that a shift in a balance between “allergy-promoting” Th-2 cells and “infection-fighting” Th-1 cells may be involved in the onset of allergy. Th-1 cells generate interferon (IF)-γ, interleukin (IL)-2, and tumor necrosis factor (TNF)-β, while Th-2 cells generate IL-4, IL-5, IL-6, IL-10, and IL-13. Current therapeutic and prophylactic strategies of vaccine design for allergy are geared towards restoration of immune regulation by promoting the development of Th-1 or T regulatory (Tr) cells that are able to down-regulate the Th-2 effector phase.

The first time an allergy-prone individual is exposed to an allergen, his or her immune system generates large amounts of the corresponding IgE antibody. These IgE molecules bind to high affinity Fc receptors (FcεRI) on the surfaces of mast cells (in tissue) or basophils (in the circulation). A subsequent exposure to an allergen causes the allergen to bind and crosslink these IgE molecules, resulting in a stimulation of the respective e.g. mast cells. This causes a rapid release of, among others, histamine and of newly formed mediators such as prostaglandins and leukotrienes.

The most important mite species as indoor allergen source in both Europe and Australia, as well as worldwide, is Dermatophagoides pteronyssinus (Der p). Major allergens of this species are the proteins Der p 1 and Der p 2. In tropical and subtropical regions the most relevant mite species is Blomia tropicalis (Bt). In these latter regions mite polysensitization to Der p 1, Der p 2 and Blo t 5 is highly prevalent. Present treatments include the use of steroids for symptomatic relief and immunotherapy using crude mite extracts, both having problem with regards to efficacy and compliance. As such there is a constant need for improved and effective therapeutic strategies.

Currently the most important aspect in the management of mite allergy is a reduction of the exposure to the mites. This includes the reduction of domestic temperature and humidity levels, the use of high filtration vacuum cleaners, frequent washing of bedding, and avoidance of matter that tends to collect dust such as wall hangings, carpets, books, etc. Current therapies include the use of histamine H-receptor antagonists, decongestants, or a combination of both. Antagonists and inverse agonists of the Histamine H-receptor, in particular the H₁-receptor, so called “antihistamines”, interfere with the action of histamine, which is released from mast cells and basophils once an allergen has bound to surface IgE on mast cells and basophils. However, antihistamines are only efficacious if administered prior to the allergen-challenge.

Decongestants relieve the swelling of nasal membranes by narrowing the blood vessels that supply the nose membranes lining. They therefore reduce one of the symptoms associated with allergies (and colds), the stuffiness of the nose, without addressing mechanisms underlying the allergic reaction. Nasal sprays such as topical nasal steroids and cromolyn sodium also can be used to treat allergy symptoms. Immunotherapy (also known as desensitization or allergy shots) is the application of small but increasing amounts of allergen at regular intervals. It is believed to increase the tolerance of the immune system to the respective allergen. Immunotherapy has been found effective to varying degrees. Its usefulness has however been limited by the potential for adverse effects, particularly anaphylaxis, and the relatively crude nature of the allergen extracts that are available. In an attempt to overcome these problems, naturally occurring isoforms of allergens from plants and trees have been shown to have a reduced capacity of being bound by IgE as a result of the substitution or deletion of amino acids.

Additionally, suppressive effects of lactic acid-producing bacteria on the development of allergy have been reported. Heat-killed L. paracasi 33 and L. acidophilus have been found to reduce symptoms of allergic rhinitis (Peng, G-C et al., Pediatric Allergy and Immunology, (2005), 16, 433-438; Ishida, Y et al., J. Dairy Sci. (2005), 88, 527-533). Administration of L. paracasei GM-080 has been reported to reduce IgE levels in mice that inhaled purified Der p 5 (U.S. Pat. No. 6,994,848). It has been suggested that a combined application of L. casei casei and dextran, but not L. casei casei alone, may prevent an increase in pollen-specific IgE, activation regulated chemokine (TARC), and interferon γ (IFN-γ) levels (Ogawa, T. et al., FEMS Immunol. Med. Microbiol. (2006), doi: 10.1111/j.1574-695X.2006.00046.x). Murooka et al. transformed L. casei K95-5 and L. plantarum NCL21 with a vector encoding the allergens Der f 1 and Der f 7 (JP 2002-281966). Kruisselbrink et al. (Clin. Exp. Immunol. [2001], 126-128) furthermore examined the effect of intranasal administration of recombinant L. plantarum 256 that expresses an immuno-dominant T-cell epitope of Der p 1 to C57BL/6 mice. An induction of peptide specific T-cell proliferation with some Th-1 properties in addition to a reduction in the Th-2 cytokine IL-5 in treated mice was observed.

Present treatments of mite allergy are thus unsatisfactory and disease prevention is not possible; thus there is a constant need for improved and effective therapeutic and prophylactic strategies.

Accordingly it is an objective of the present invention to provide a means suitable for modulating the immune response in mite allergy. This objective is solved by the subject matter of the appending independent claims.

### SUMMARY OF THE INVENTION

In one aspect the present invention provides a recombinant lactobacillus. The recombinant lactobacillus includes a heterologous nucleic acid sequence. This nucleic acid sequence encodes at least an immunogenic fragment of any one mite allergen of Der p 1, Der p 2, and Blo t5, or an immunogenic homolog thereof. The nucleic acid sequence may thus encode an entire mite allergen, or a respective immunogenic homolog thereof. The recombinant lactobacillus is also provided for use in therapy.

In a further aspect the invention provides a pharmaceutical composition. The pharmaceutical composition includes a recombinant lactobacillus as described above, and a pharmaceutically acceptable carrier or diluent.

In another aspect the invention provides a pharmaceutical kit. The pharmaceutical kit includes a composition as described above. It further includes an allergen or an immunogenic fragment thereof.

In a further aspect the invention provides a method of modulating the immune response to an allergen in a mammal. The method includes administering a composition as described above.

In yet a further aspect the invention relates to the use of a recombinant lactobacillus as described above in the manufacture of a pharmaceutical composition and a pharmaceutical kit for modulating the immune response to an allergen.

### BRIEF DESCRIPTION OF THE DRAWINGS

The invention will be better understood with reference to the detailed description when considered in conjunction with the non-limiting examples and the accompanying drawings, in which:

FIG. 1 depicts schematically a lactobacillus/E. coli shuttle vector (A) and an intermediate vector used to generate a dust mite allergen expression construct.

FIG. 2 depicts schematically a further lactobacillus/E. coli shuttle vector.

FIG. 3 shows a further expression vector that can be included in a recombinant lactobacillus of the present invention.

FIG. 4 shows another expression vector that can be included in a recombinant lactobacillus of the present invention.

FIG. 5 shows the Western immunoblot detection of heterologous expression of Der p 2 in two strains of lactobacilli, L. casei Shirota and L. rhamnosus gg.

FIG. 6 shows the translocation of L. casei Shirota-eGFP into both T- and B-cell region of Peyer's patches.

FIG. 7 shows the translocation of intact L. casei Shirota-eGFP into the vacoules of mono- and polymorphic cells in Peyer's Patches by transmission electron microscopy.

FIG. 8 shows the induction of TGF-β production in T-cells co-cultured in-vitro with L. casei Shirota.

FIG. 9 depicts the increase in Der p 2-specific T-cells proliferation and regulatory CD4⁺CD25⁺ T-cells in mice fed with recombinant Lc/Dp2.

FIG. 10 depicts a prophylactic regimen used in animal studies.

FIG. 11 shows Der p 2-specific immunoglobulin responses.

FIG. 12 depicts a cytokine profile of spleen T-cells.

FIG. 13 depicts a cytokine profile of mesenteric lymph node (MLN) cells.

FIG. 14 shows the profiles of cytokines of the broncholalveolar lavage fluid (BALF) in mice.

FIG. 15 depicts BALF analysis and lung histology in mice.

FIG. 16 depicts a therapeutic regimen used in animal studies.

FIG. 17 depicts Der p 2-specific immunoglobulin responses in mice.

FIG. 18 shows a profile of selected cytokines in spleen T-cells.

FIG. 19 shows a profile of selected cytokines in mesenteric lymph nodes cells.

FIG. 20 depicts the pathophysiological changes in the lungs and a broncholalveolar fluid analysis.

FIG. 21 shows the profiles of cytokines of the BALF.

FIG. 22 depicts a treatment model of mice presensitized with Der p 2 allergen.

FIG. 23 illustrates the systemic immunoglobulin response and T cell cytokines.

FIG. 24 shows a treatment model hypothesis.

FIG. 25 shows a schematic of the experimental protocol for the analysis of the effect of subcutaneous priming of on mice (without application of adjuvant).

FIG. 26 depicts the Kinetics of Der p 2-specific humoral response in mice.

FIG. 27 depicts an RT-PCR analysis on cytokine profiles of splenic CD4⁺ T-cells.

FIG. 28 depicts cytokine profiles of lymph nodes in culture.

FIG. 29 shows cytokine profiles of SP cultures.

FIG. 30 depicts the proliferation and cytokine response of antigen-specific TH2 cells upon co-culture with CD4⁺ CD25⁺ cells.

FIG. 31 depicts a regimen used in animal studies with recombinant L. casei Shirota expressing the Blo t 5 allergen.

FIG. 32 depicts the analysis of Blo t 5-specific serum immunoglobulins by ELISA (cf. also FIG. 31).

FIG. 33 depicts the cytokine analysis of mice sacrificed in the regimen depicted in FIG. 34.

FIG. 34 depicts the nucleic acid sequence and the amino acid sequence of the allergen Blo t 5 in the expression vector pLP400.

FIG. 35 depicts the nucleic acid sequence and the amino acid sequence of the allergen Der p 2 in the expression vector pLP500.

Table I depicts TH2 cytokine profiles determined of splenic CD4⁺ T-cells by real-time PCR.

### DETAILED DESCRIPTION OF THE INVENTION

The present invention provides a recombinant lactobacillus. Lactobacilli are well known gram positive bacteria that vary in morphology from long, slender rods to short coccobacilli, which frequently form chains. The recombinant lactobacillus of the present invention may be any lactobacillus. Currently 91 species of the genus lactobacillus are known. Examples of a respective lactobacillus include, but are not limited to, Lactobacillus casei, Lactobacillus acidophilus, Lactobacillus fermentum, Lactobacillus gasseri, Lactobacillus pentosus, Lactobacillus plantarum, Lactobacillus sporogenes, Lactobacillus brevis, Lactobacillus delbrueckii, Lactobacillus salivarius, Lactobacillus hilgardii, Lactobacillus lactis, Lactobacillus rhamnosus, Lactobacillus johnsonii, Lactobacillus leishmanis, Lactobacillus jensenii, Lactobacillus reuteri, Lactobacillus sakei, Lactobacillus curvatus, Lactobacillus cellobiosus, Lactobacillus crispatus, Lactobacillus caucasicus, and Lactobacillus helveticus, to name a few. While many lactobacilli, such as Lactobacillus reuteri, Lactobacillus casei, Lactobacillus plantarum, and Lactobacillus acidophilus are able to colonize the gastrointestinal tract, i.e. “implantable”, some lactobacilli such as L. delbrueckii bulgaricus and L. lactis are considered transient, non-implanting flora-. The present invention applies to any respective strain of lactobacillus of the present invention, whether implantable or not. For convenience of use (cf. below) it may in some embodiments be desired to select a strain that is able to implant.

Any subspecies and strain of a respective lactobacillus may be used. As an illustrative example, there are several known subspecies of L. casei, such as L. casei subspecies casei, L. casei subspecies paracasei. Examples of strains of Lactobacillus casei include L. casei strain KE99, L. casei strain CRL 431, L. casei strain BL155, L. casei strain Shirota, and L. casei N19. Examples of strains of Lactobacillus rhamnosus include, but are not limited to, L. rhamnosus strain MTCC 1408, L. rhamnosus strain HN001, L. rhamnosus strain Lcr35 and L. rhamnosus strain GG. L. rhamnosus GG also known Lactobacillus GG (Gorbachi & Goldini) was initially classified under L. acidophilus. It has been suggested to be classified as a strain of L. casei and also been proposed to be reclassified as a unique species L. zeae. In the following it will be referred to as L. rhamnosus GG.

The recombinant lactobacillus includes a heterologous nucleic acid sequence encoding at least an immunogenic fragment of a mite allergen, or an immunogenic homolog thereof. Accordingly, the respective nucleic acid sequence corresponds to the amino acid sequence of a polypeptide. Therefore, the at least immunogenic fragment of the mite allergen encoded by the heterologous nucleic acid sequence includes, or is, a polypeptide. By “fragment” in reference to an allergen is meant any amino acid sequence present in a polypeptide of a respective allergen. In some embodiments the term “fragment” refers to the absence of posttranslational modifications, such as a saccharide or saccharide chain, which are present in a respective naturally occurring allergen. In such embodiments the amino acid sequence of a respective allergen fragment may be of any length, whether the entire length or a part of the full length sequence of any naturally occurring form, including a variant, of the allergen. In other embodiments the term “fragment” refers to any amino acid sequence present in a polypeptide of a respective allergen that is shorter than the full length sequence of a naturally occurring form of the allergen. The naturally occurring form of a respective allergen is understood to be a mature full-length protein that is typically derived from a precursor protein. As an illustrative example, of the allergen Der p 1a preproenzyme, termed DERP1_DERPT, is known to exist in vivo, which is a precursor of the UniProtKB/Swiss-Prot accession number P08176 (secondary accession number Q24616) and the NCBI accession number AAB60215. This precursor is of 320 amino acids, whereas the mature and fully active allergen Der p 1 of the NCBI accession numbers 2AS8_A and 2AS8_B is of 222 amino acids. Similar precursors are known of other mite allergens, such as the allergen Der p 2 precursor (Der p II) (DPX) of the UniProtKB/Swiss-Prot accession number P49278 termed ALL2_DERPT. In such embodiments posttranslational modifications that are found in a respective naturally occurring allergen may be present in a fragment to any degree.

In some embodiments the nucleic acid sequence included in the lactobacillus of the invention encodes a mite allergen fragment, which includes at least 8% of the amino acid sequence of the naturally occurring, i.e. the mature full-length protein, form of the respective mite allergen. As an illustrative example, the recombinant lactobacillus may include a heterologous nucleic acid sequence encoding at least an immunogenic fragment of the mite allergen Der p 1 (or a respective immunogenic homolog thereof). The fragment may in this case include at least 8% of the amino acid sequence of Der p 1. In some of these embodiments such a fragment may thus include any part or parts of the amino acid sequence that correspond(s) to 8-100% of the entire amino acid sequence of a naturally existing mite allergen, such as 10-100%, for example 15-100%, including 25-100%. In some embodiments such a fragment may thus include a part that is an immunogenic homolog (cf. below) of a respective part of the amino acid sequence of a naturally existing mite allergen.

The term “heterologous” when used in reference to a nucleic acid sequence or molecule, means a nucleic acid sequence not naturally occurring in the respective bacillus or cell, into which the nucleic acid molecule has been (or is being) introduced. A heterologous nucleic acid sequence thus originates from a source other than the respective bacillus or cell and can occur naturally or non-naturally. A respective heterologous nucleic acid sequence may for example be integrated into the lactobacillus chromosome or into any other nucleic acid molecule that is present in the lactobacillus, such as a vector (cf. also below) or an RNA molecule.

Mite allergens are divided into specific groups based on their biochemical composition, sequence homology, and molecular weight. The designation for a characterized allergen is the first three letters of the genus, the first letter of the species name, and a final number. The final number designates the order in which the allergen was isolated or the number for other already characterized allergens to which it is homologous. Mite allergens encoded completely, as a fragment thereof, or in form of a respective immunogenic homolog, by the heterologous nucleic acid sequence, which is included in a lactobacillus of the present invention, are typically Der p 1, Der p 2, and Blo t 5. As an illustrative example, the recombinant lactobacillus may be Lactobacillus casei, and the heterologous nucleic acid sequence included therein may encode the mite allergen Der p 2.

As already indicated above, the heterologous nucleic acid sequence included in the recombinant lactobacillus of the invention encodes at least an immunogenic fragment of a mite allergen, or an immunogenic homolog thereof. The term “allergen” as used herein refers to a molecule that is capable of inducing an allergy in an individual or an animal. As already described above, an allergen is capable of inducing an immune response, i.e. to stimulate lymphocytes to produce antibody or to attack the allergen directly. Accordingly an allergen is an antigen that is recognized by the immune system and may cause an allergic reaction. Such an allergic reaction may be caused by any form of direct contact with the allergen such as ingestion, e.g. eating or drinking, inhalation, or direct contact, e.g. via the skin. Typically an antigen, including an allergen, it is a polypeptide such as a protein, or a polysaccharide. In the context of the present invention the term antigen refers to any polypeptide, which may include any modification such as a saccharide or a lipid. It also refers to short peptides known as haptens, which are typically coupled to a carrier molecule of larger size than the hapten, e.g. a protein, or to a cell.

The term “immunogenic”, as used herein refers to the capability of matter of evoking an immune response, i.e. of being immunologically active. Accordingly, when used in the context of a fragment of an allergen, a respective fragment may in some embodiments in itself be able to cause an immune response, for instance when administered to an individual or animal. It should however be noted that an immunogenic fragment of an allergen need not in itself possess the capability of evoking an immune response. Its capability of being immunologically active may in some embodiments rather depend on the fragment being coupled to additional matter. In some embodiments this coupling to additional matter may for instance occur in vivo, for example by binding to a protein. Thus in some embodiments an immunogenic fragment of an allergen includes, or is, a hapten, which needs to be coupled to a carrier molecule or to a cell in order to show its immunogenic properties. Where an immunogenic fragment of an allergen is included in a heterologous nucleic acid sequence, it may therefore be of any sequence length or size, as long as an obtained peptide (transcribed and translated in vitro, ex vivo or in vivo) is capable of evoking an immune response, whether in itself or when coupled to additional matter. In typical embodiments the mite allergen or the fragment thereof is capable of binding to at least one IgE antibody. A respective antibody may for instance by an antibody of an individual or an animal being allergic or sensitive to mites, such as dust mites. Generally, a respective fragment of an allergen includes at least one epitope. In some embodiments, however, two or more fragments of an allergen may need to be combined to obtain a respective epitope, for instance when coupled to the same carrier molecule. An epitope, also called antigenic determinant, is a part of an antigen molecule—in this case an allergen molecule—that can be recognized and bound by an antigen-binding site of an antibody or by a T-cell receptor. Different antibodies and T-cell receptors bind to different epitopes of an antigen. Two epitopes of Der p 2 are for instance known, to which T cells from Japanese patients with allergic rhinitis are able to bind, two further epitopes are known to be bound by T helper cells from the same patients.

The term “immunogenic homolog” as used herein when used in reference to a mite antigen or an immunogenic fragment thereof, means a polypeptide having a high degree of homology to a respective naturally existing mite antigen and which can be specifically recognized and bound by at least one antibody or T-cell receptor that is active against the corresponding naturally existing mite antigen. In typical embodiments, such a fragment may have at least 60% sequence identity with the corresponding amino acid sequence of a naturally existing mite antigen (including an immunogenic fragment thereof). In some embodiments, a respective fragment has at least 80%, such as at least 85%, at least 90% or at least 95% sequence identity with the corresponding amino acid sequence of a naturally existing mite antigen. By “sequence identity” is meant a property of sequences that measures their similarity or relationship. This term refers to the percentage of pair-wise identical residues obtained after a homology alignment of an amino acid sequence, or a nucleic acid sequence, of a known mite antigen with an amino acid or a nucleic acid sequence, respectively, in question, wherein the percentage figure refers to the number of residues in the longer of the two sequences.

Also encompassed by the present invention are nucleic acid sequences substantially complementary to the above nucleic acid sequence. “Substantially complementary” as used herein refers to the fact that a given nucleic acid sequence is at least 90, for instance at least 95, and in some embodiments 100% complementary to another nucleic acid sequence. The term “complementary” or “complement” refers to two nucleotides that can form multiple favourable interactions with one another. Such favourable interactions include Watson-Crick base pairing. A nucleotide sequence is the complement of another nucleotide sequence if all of the nucleotides of the first sequence are complementary to all of the nucleotides of the second sequence.

In some embodiments the recombinant lactobacillus is furthermore capable of expressing the at least immunogenic fragment of a mite allergen, or a respective immunogenic homolog thereof. In such embodiments the respective sequence, encoding the allergen, fragment or immunogenic homolog thereof, may be operably linked to a promoter effective to initiate transcription in a host cell. The recombinant nucleic acid can also contain a transcriptional initiation region functional in a cell, a sequence complementary to an RNA sequence encoding a kinase polypeptide and a transcriptional termination region functional in a cell. In one embodiment the recombinant lactobacillus expresses the mite allergen, or a fragment thereof, or a respective immunogenic homolog.

The recombinant lactobacillus may be obtained from a naturally occurring lactobacillus by introducing the heterologous nucleic acid sequence therein. As an illustrative example, the sequence encoding at least an immunogenic fragment of a mite allergen, or an immunogenic homolog thereof, may be included in a heterologous nucleic acid molecule, such as a heterologous polynucleotide. A nucleic acid molecule encoding an allergen of the invention and an operably linked promoter may be introduced into the lactobacillus either as a nonreplicating DNA or RNA molecule, which may either be a linear molecule or a closed covalent circular molecule. As an illustrative example, a DNA molecule may be stably integrated into chromosome of the lactobacillus. A vector may be employed which is capable of integrating the desired gene sequence into the lactobacillus chromosome. As an illustrative example, the use of the plasmid pAMβ1 to integrate the gene for L-lactate dehydrogenase into the chromosome of Lactobacillus delbrueckii by replacing the gene of D-lactate dehydrogenase has been disclosed in U.S. Pat. No. 5,747,310. Where desired, lactobacilli which have stably integrated the introduced DNA into their chromosome can be selected by also introducing one or more markers which allow for selection of host cells which contain the expression vector.

The term “nucleic acid molecule” as used herein refers to any nucleic acid in any possible configuration, such as single stranded, double stranded or a combination thereof. Nucleic acids include for instance DNA molecules (e.g., cDNA or genomic DNA), RNA molecules (e.g., mRNA), analogues of the DNA or RNA generated using nucleotide analogues or using nucleic acid chemistry, and PNA (protein nucleic acids). DNA or RNA may be of genomic or synthetic origin and may be single or double stranded. In the present invention typically, but not necessarily, an RNA or a DNA molecule is included in the recombinant lactobacillus. Such nucleic acid molecule can be e.g. mRNA, cRNA, synthetic RNA, genomic DNA, cDNA synthetic DNA, a copolymer of DNA and RNA, oligonucleotides, etc. A respective nucleic acid molecule may furthermore contain non-natural nucleotide analogues and/or be linked to an affinity tag or a label (cf. above).

Many nucleotide analogues are known and can be present in nucleic acid sequence included in the lactobacillus of the invention. A nucleotide analogue is a nucleotide containing a modification at for instance the base, sugar, or phosphate moieties. As an illustrative example, a substitution of 2′-OH residues of siRNA with 2′F 2′O-Me or 2′H residues is known to improve the in vivo stability of the respective RNA. Modifications at the base moiety include natural and synthetic modifications of A, C, G and T/U different purine or pyrimidine bases, such as uracil-5-yl, hypoxanthin-9-yl and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

In some embodiments the heterologous nucleic acid may be included in a heterologous nucleic acid molecule, e.g. a plasmid or vector, that does not integrate into a lactobacillus chromosome. Accordingly, in this case the recombinant lactobacillus includes a heterologous nucleic acid molecule in addition to its chromosome. A respective vector may contain one or more regulatory sequences, such as a promoter, an enhancer, a silencer or a terminator sequence. Such regulatory sequences may control, e.g. facilitate, replication of the vector or transcription and/or translation of encoded sequences. An illustrative example of a respective vector is an expression vector. In some embodiments the allergen may be under the control of an inducible promoter. As an illustrative example, an expression system controlled by the antimicrobial peptide nisin has been used in Lactobacillus plantarum (Pavan, S. et al., Applied and Environmental Microbiology (2000) 66, 4427-4432). In other embodiments the allergen may be expressed in a constitutively active manner. As an illustrative example, constitutive expression of a fusion protein of proteinase PrtB and the tetanus toxin mimotope under the control of the PrtB promoter of Lactobacillus delbrueckei bulgaricus in Lactobacillus johnsonii has been disclosed by Scheppler et al. (Vaccine [2002] 20, 2913-2920).

The term “vector” relates to a single or double-stranded circular nucleic acid molecule that can be introduced, e.g. transfected, into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding an allergen or a fragment thereof can be inserted into a vector by cutting the vector with restriction enzymes and ligating the two pieces together. Numerous vectors have for example been developed based on cryptic plasmids that originate from lactic acid producing bacteria (for an overview cf. Shareck et al., Critical Reviews in Biotechnology (2004), 24, 155-208). Cryptic plasmids are extrachromosomal DNA elements that do not have any apparent function, i.e. encode no recognizable phenotype, besides their replication function. Sigma-replicating and theta-replicating plasmids are the most common plasmids in lactic acid producing bacteria.

Examples of cryptic plasmids originating from Lactobacillus plantarum include, but are not limited to, pcaT, pA1, pLP1, p 8014-1, pC30il, pLB4, pLP2000, pLP9000, pLKL, pLKS and pMD5057. Examples of vectors originating from Lactobacillus fermentum include, but are not limited to, pLY2, pLY4, pLEM3, pLF1311, and pKC5b. Examples of cryptic plasmids originating from Lactobacillus acidophilus include, but are not limited to, p1, p3, pPM4, pLA103, pLA105, and pLJ106. Numerous further cryptic plasmids from other Lactobacillus species are known in the art (cf. e.g. Shareck et al., supra). Any of the aforementioned plasmids may be used to generate a vector, including an expression vector, to obtain a lactobacillus according to the present invention. Examples of cryptic plasmids originating from Lactobacillus plantarum include, but are not limited to, pcaT, pA1, pULP8, pULP9, pLP825, pLP82H, pLPC37, pPSC1, pPSC11, pPSC22, pLPV106, pLPIII, pLEM5, pLEM7, and pLFVM2, to name just a few. Examples of cryptic plasmids originating from Lactobacillus fermentum include, but are not limited to, pLY2, pLY4, pLEM5, pLEM7, pLFVM2, and pSP1.

A cryptic plasmid such as for instance one of the above examples, may for instance serve in the construction of a shuttle vector that can be used to obtain the recombinant lactobacillus of the present invention. As an illustrative example, the cryptic plasmid pLC494 isolated from Lactobacillus casei has been used to construct a Lactobacillus/E. coli shuttle vector (pJLE4941) by genetic engineering technology using isolated plasmid pLC494 and isolated C. perfringens/E. coli plasmid pJIR418 (An, H-Y, Miyamoto, T., Plasmid (2006) 55, 128-134). A respective shuttle vector can replicate in both respective host species, i.e. E. coli and a lactobacillus. Another illustrative example of a Lactobacillus/E. coli shuttle vector is the plasmid pLE16 constructed from the L. delbrueckii bulgaricus plasmid pLB10 and the E. coli plasmid pBR328. Two further illustrative examples of a expression vector originating from a cryptic plasmid are pLP400 and pLP500, two further Lactobacillus/E. coli shuffle vectors. As yet another example, the E. coli expression vector pLF22 has been adapted for use in lactobacilli by including a replicon of the cryptic plasmid pLF1311 from Lactobacillus fermentum.

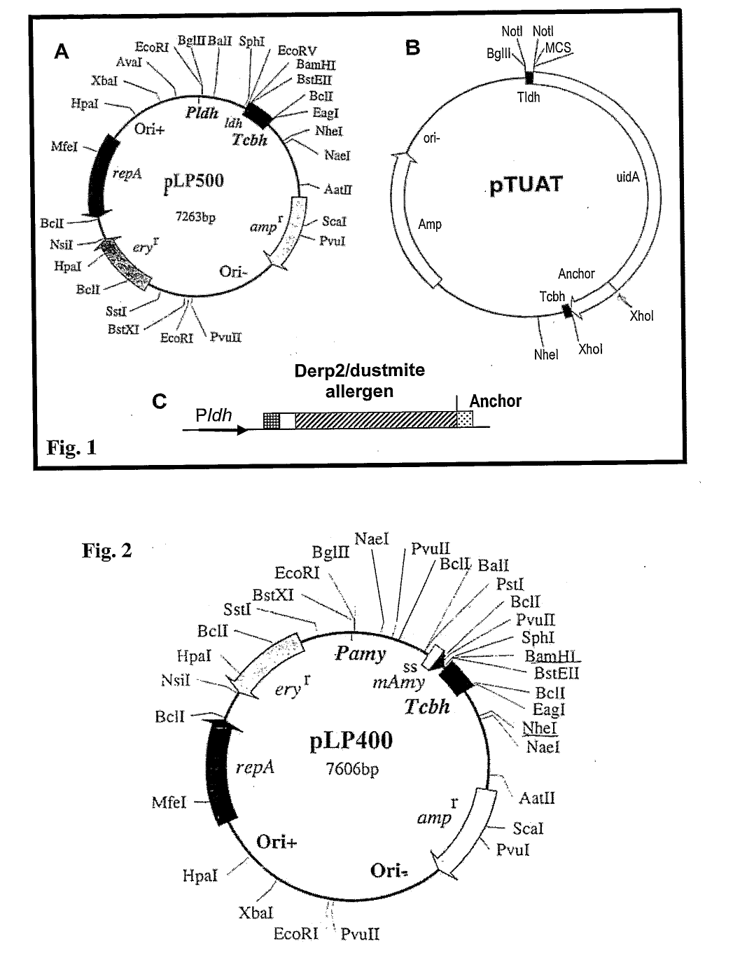

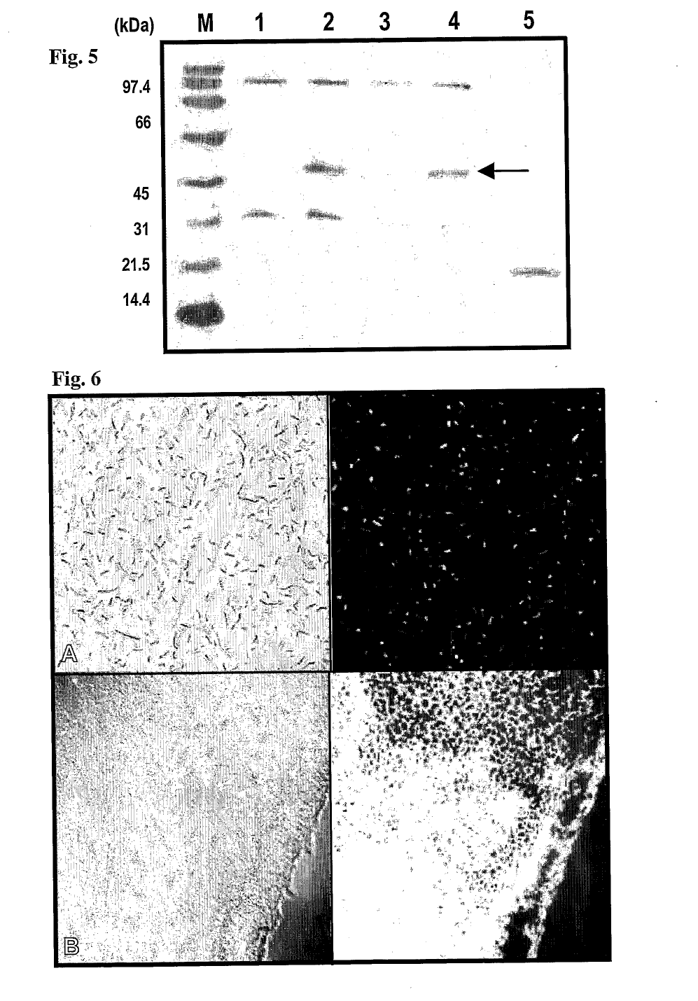

As an illustrative example of obtaining a recombinant lactobacillus according to the present invention, the mite allergen gene Der p2 can be genetically engineered and cloned into the pL500 lactobacillus/E. coli shuttle vector (cf. FIG. 1 and the following examples) or the corresponding vector pL400. The pLP400 and pLP500 shuttle vectors contain expression signals and replication elements derived from lactobacillus DNA sequences. These recombinant vectors pLP400 and pL500 carrying the Der p2 gene can be introduced into a lactobacillus. Various methods of introducing nucleic acids into bacilli are known to those in the art, such as transformation, transfection, injection or electroporation. A respective vector may e.g. be electroporated into a lactobacillus such as L. casei Shirota (L.c) or L. rhamnosus gg (L.gg). FIG. 5 illustrates the detection of the intracellular expression of Der p 2 in these two strains, following electroporation, by means of a Western immunoblot (FIG. 5).

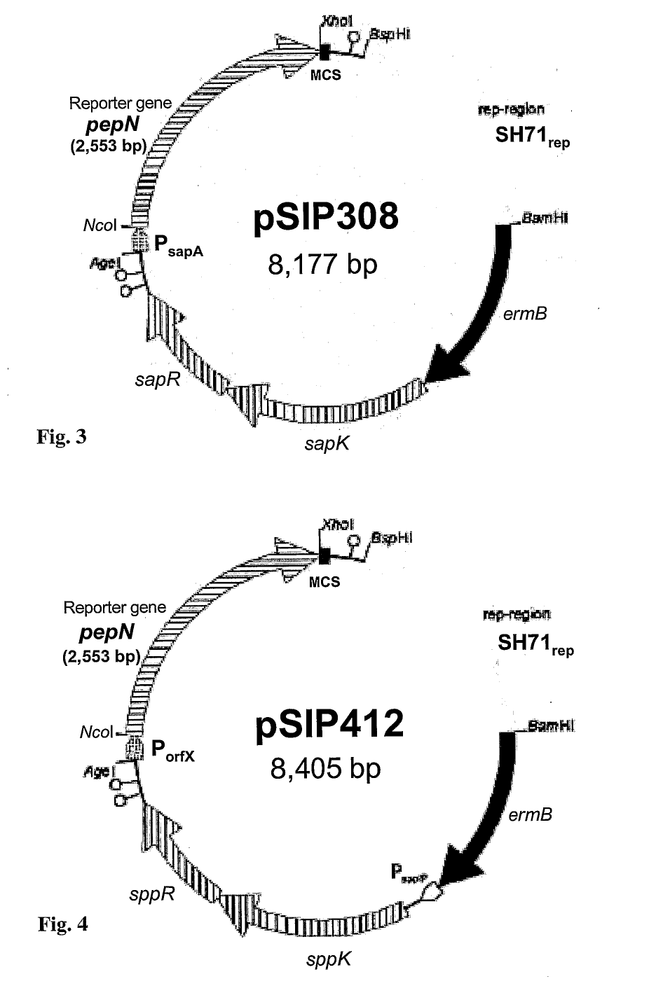

As another illustrative example, the lactobacillus expression vector pSIP308, a vector obtained from the plasmid pSIP300 or the lactobacillus expression vector pSIP412, a vector obtained from the plasmid pSIP401, may likewise be genetically engineered and introduced into a lactobacillus (cf. FIG. 3 and FIG. 4). Sørvig et al. (Microbiology (2005) 151, 2439-2449) have recently disclosed the generation of these and other related expression vectors as well as their use in L. plantarum and L. sakei. Those skilled in the art will be aware of the fact that some modifications may be required in order to use these vectors for other lactobacilli such as e.g. L. casei or L. fermentum.

Typically the mite allergen is Der p 1, Der p 2 or Blo t 5 (cf. also below). The allergen Der p 1 may for instance be encoded by the 1099 base pair sequence of the NCBI accession number U11695. It may also be encoded by a sequence that is or includes the 650 base pair sequence of the NCBI accession number AY947536 or the 591 base pair sequence of the NCBI accession number AF276239.

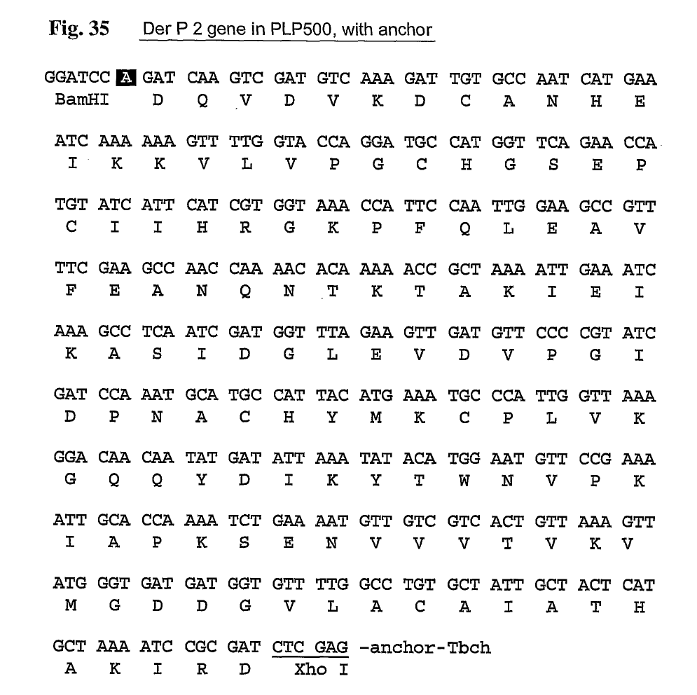

In some embodiments where the mite allergen is Der p 2, this allergen is encoded by the sequence of SEQ ID NO: 1 (cf. FIG. 35). In some embodiments where the mite allergen is Blo t 5, this allergen is encoded by the sequence of encoded by a sequence that is or includes the 537 base pair sequence of the NCBI accession number U59102. In some embodiments where the mite allergen is Blo t 5, the at least immunogenic fragment of the mite allergen is encoded by the sequence of SEQ ID NO: 2 (cf. FIG. 34). The sequence of SEQ ID NO: 2 encodes a C-terminal fragment of 117 amino acids of the 134 amino acid sequence of the UniProtKB/Swiss-Prot accession number O96870 (secondary accession number Q17283; corresponding to NCBI accession numbers O96870 and AAD10850). In one respective embodiment the heterologous nucleic acid includes the sequence of SEQ ID NO: 1 or of SEQ ID NO: 2, respectively. In other embodiments the heterologous nucleic acid includes a functional equivalent of SEQ ID NO: 1 or of SEQ ID NO: 2. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or the entire amino acid sequence obtained from the nucleic acid sequence of SEQ ID NO: 1 or of SEQ ID NO: 2 can be transcribed by a nucleic acid sequence significantly different from that shown in SEQ ID NO: 1 or in SEQ ID NO: 2. Nevertheless, the encoded amino acid sequence thereof is preserved.

In addition, the nucleic acid sequence may include a nucleotide sequence which results from the addition, deletion or substitution of at least one nucleotide to the 5′-end and/or the 3′-end of the nucleic acid sequences of SEQ ID NO: 1 and of SEQ ID NO: 2, or a derivative thereof. Any nucleotide or polynucleotide may be used in this regard, provided that its addition, deletion or substitution does not abolish the immunogenic properties of the respective transcribed polypeptide. As an illustrative example, the nucleic acid molecule encoding at least an immunogenic fragment of a mite allergen or an immunogenic homolog thereof, may, as necessary, have restriction endonuclease recognition sites added to its 5′-end and/or 3′-end.

In some embodiments the mite allergen is Der p 2, and the heterologous nucleic acid sequence encodes an immunogenic homolog of Der p 2. This heterologous nucleic acid sequence may for example have a nucleic acid sequence of at least 80% identity to the nucleic acid sequence of SEQ ID NO: 1 (cf. above). In some of these embodiments the heterologous nucleic acid sequence may have a nucleic acid sequence of at least 90% identity to the nucleic acid sequence of SEQ ID NO: 1, such as an identity of at least 95%. Correspondingly, in some embodiments the mite allergen is Blo t 5, and the heterologous nucleic acid sequence encodes an immunogenic homolog of Blo t 5. This heterologous nucleic acid sequence may for example have a nucleic acid sequence of at least 80% identity to the nucleic acid sequence of SEQ ID NO: 2. In some of these embodiments the heterologous nucleic acid sequence may have a nucleic acid sequence of at least 90% identity to the nucleic acid sequence of SEQ ID NO: 2, such as an identity of at least 95%.

The present invention also features the recombinant lactobacillus as described above for use in therapy. An illustrative example of a respective therapy is the modulation of the immune response to an allergen as described in detail below. In embodiments for use in therapy the recombinant lactobacillus is provided in a form in which it is suitable for being administered to an organism, e.g. a mammal such as a human. As an illustrative example, the recombinant lactobacillus may be provided as included in food. Examples of respective forms of food including the recombinant lactobacillus include, but are not limited to yogurt, sauerkraut, pickles, Korean kimchi, cheese, buttermilk sourdough bread and silage.

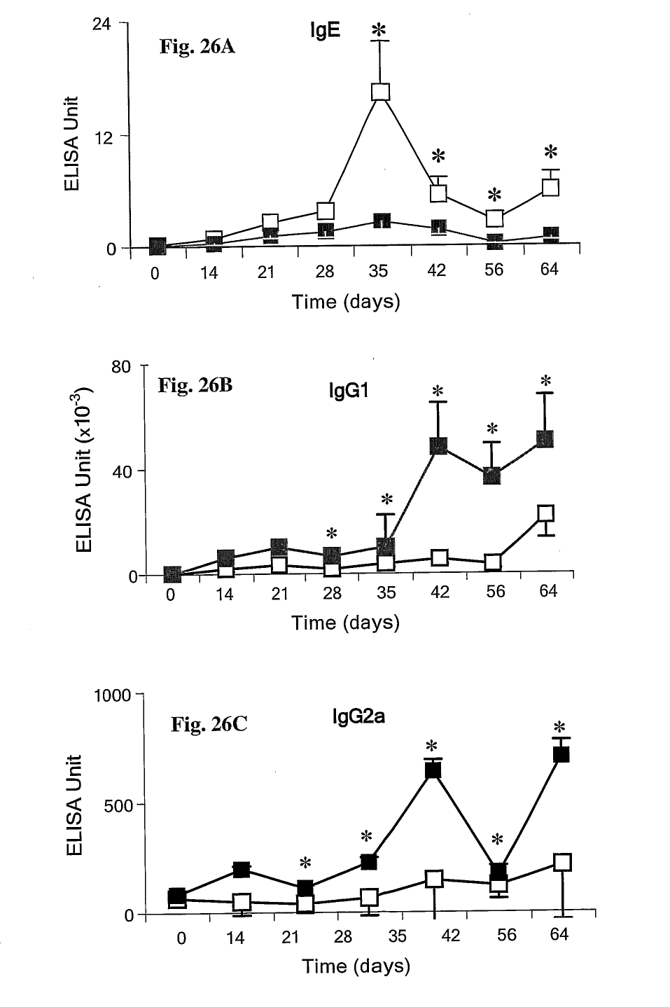

A further example of a respective form suitable for administration is a pharmaceutical composition. A pharmaceutical composition according to the present invention includes a recombinant lactobacillus as described above. The recombinant lactobacillus may be of any activity status. It may for instance be alive and fully vivid, metabolizing and replicating (cf. e.g. FIG. 25 and FIG. 26). As a further example, the recombinant lactobacillus may, at least to a degree, be inactivated, e.g. by heat treatment. A heat inactivation may for instance be desired in order to destroy heat-labile complement proteins. In some embodiments the recombinant lactobacillus may be dead. In some of these embodiments the recombinant lactobacillus may thus be intact, whether alive or dead. In other embodiments it may be disintegrating or disintegrated. The structure of the recombinant lactobacillus may for instance be partly or entirely collapsed, including the presence of cell debris to any degree.

In some embodiments the pharmaceutical composition includes a therapeutically effective amount of the recombinant lactobacillus. As used herein, the phrase “therapeutically effective amount” refers to an amount of the recombinant lactobacillus which will, at dosages and for periods of time necessary, achieve a desired therapeutic result. It may for instance relieve or alleviate fully or at least to some extent one or more of the symptoms of the allergic condition being treated when being administered. The precise therapeutically effective amount of the recombinant lactobacillus will depend on a number of factors including, but not limited to, the disease state, the age, sex and weight of the subject being treated, the sensitivity of the subject to the respective allergen, and the severity of the allergy, the nature of the formulation, and the route of administration, and will ultimately be at the discretion of the attendant physician or veterinarian. Dosage regimens may be adjusted to provide the optimum therapeutic response. Typically, the recombinant lactobacillus will be given for treatment in the range of about 10⁹ to about 10¹¹ CFU (colony forming units) per recipient (animal) per day, such as in the range of about 5×10¹⁰ CFU per day. Acceptable daily dosages may be from about 10⁹ CFU to about 10¹¹ CFU per recipient (animal)/day, and in particular from about 5×10¹⁰ CFU to about 5×10¹¹ CFU/day, for example. It is understood that different quantities of the pharmaceutical composition may be administered in order to achieve an effective amount of administration, or that a pharmaceutical composition with an adapted relative amount of the lactobacillus may be used for administration. As an illustrative example, the pharmaceutical composition may include an amount in a range that is suitable to achieve a daily dose of about 5×10¹⁰ CFU to about 5×10¹¹ CFU of the recombinant lactobacillus as a therapeutically effective amount when administered.

In some embodiments the pharmaceutical composition includes a prophylactically effective amount of the recombinant lactobacillus. As used herein, the phrase “prophylactically effective amount” refers to an amount of the recombinant lactobacillus, at dosages and for periods of time necessary, which will achieve a desired prophylactic result, such as prevent fully or at least to some extent one or more of the symptoms of an allergic condition being treated when being administered. A prophylactically effective amount can be determined as described above for the therapeutically effective amount. For any particular subject, specific dosage regimens may be adjusted over time according to the individual need and the professional judgement of the person administering or supervising the administration of the compositions. For prophylaxis, the recombinant lactobacillus may for example be given in the range of about 10⁹ CFU to about 10¹⁰ CFU of recipient (animal) per day, such as in the range of 5×10⁹ CFU per day. As an illustrative example, the pharmaceutical composition may include an amount in a range that is suitable to achieve a daily dose of about 10⁹ CFU to about 10¹⁰ CFU of the recombinant lactobacillus as a prophylactically effective amount, when administered.

The pharmaceutical composition furthermore includes a pharmaceutically acceptable carrier, diluent or excipient. Any carrier or diluent may be employed that does not obviate the immunomodulatory activity of the recombinant lactobacillus. If desired, a carrier or diluent may be chosen that does not affect the immunomodulatory activity of the recombinant lactobacillus at all. The carrier can be a solvent or dispersion medium containing, for example, water (such as e.g. physiological saline, aqueous sodium caroboxymethyl cellulose, or aqueous polyvinylppyrrolidone, ethanol, a polyol such as glycerol, propylene glycol and liquid polyethylene glycol, suitable mixtures thereof and vegetable oils. An illustrative example of a suitable carrier are liposomes. Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical composition can additionally include for instance lubricating agents (such as talc, magnesium stearate, and mineral oil), wetting agents, emulsifying and suspending agents, preserving agents such as methyl- and propylhydroxy-benzoates, sweetening agents, and flavoring agents. A stabilizer may also be included in the pharmaceutical composition. Examples of suitable stabilizers include, but are not limited to, an alkali metal hydrogen phosphate salt, glutamate, serum albumin, gelatin, or casein. An adjuvant may also be included in the pharmaceutical composition, for example a surface active substance such as hexadecylamine, octadecylamine, an octadecyl amino acid ester, lysolecithin, dimethyl-dioctadecylammonium bromide, methoxyhexadecylgylcerol, and a pluronic polyol, polyamines, such as pyran, dextran-sulfate, poly IC, carbopol; peptides such as muramyl dipeptide, dimethylglycine, tuftsin, an oil emulsion, and a mineral gel such as aluminum hydroxide, aluminum phosphate. The adjuvant may be, for example, alum or a composition containing a vegetable oil, isomannide monooleate and aluminum mono-stearate. Further examples of an adjuvant include microparticles or beads of biocompatible matrix materials. The pharmaceutical composition may further include a preservative such as an antibacterial and antifungal agent, for example, parabens, chlorobutanol, phenol, sorbic acid, or thimerosal.

The pharmaceutical composition may for example be of solid form such as tablets or pills or of liquid form. As an illustrative example of a liquid form, a composition that includes the recombinant lactobacillus for administration orally or by injection, may be an aqueous solution, a suitably flavored syrup, an aqueous or oil suspension, or a flavoured emulsion with edible oils such as sesame oil, coconut oil, cottonseed oil, or peanut oil, or an elixir.

In some embodiments the pharmaceutical composition further includes at least one of a corticosteroid, an antihistamine, a leukotriene modifying agent, a mast cell degranulation inhibitor (mast cell stabilizer), a decongestant and a β2-adrenoceptor agonist.

Corticosteroids are generally capable of reducing or eliminating allergic inflammation. Including a corticosteroid into a pharmaceutical composition of the invention may for instance target the prevention of airway remodeling and the achievement of normal lung function in asthma. Examples of suitable corticosteroids include, but are not limited to cortisol, hydrocortisone, hydrocortisone acetate, corticosterone, dexamethasone, prednisone, methylprednisolone prednisolone, clobetasone, methylprednisolone, prednicarbate, flumetasone, fluocinolone, mometasone, betamethasone, fluocortolone, fluocinolone, amcinoid, fluocinoid, halcinoid, fluticasone and triamcinolone.

Antihistamines such as H₁-antihistamines, H₂-antihistamines or H₄-antihistamines, are molecules that are able to block the effects of histamine by binding to histamine receptors. Typically the histamine has been released during an immune response, for example from mast cells. Some molecules, such as e.g. the H₂-antihistamines cimetidine and tiotidine have been found to be inverse agonists (at the H₂-receptor). However most antihistamines are presently believed to be receptor antagonists. Examples of H₁-antihistamines include, but are not limited to, ethylenediamines such as mepyramine (pyrilamine) or antazoline, ethanolamines such as diphenhydramine, carbinoxamine, doxylamine, clemastine, or dimenhydrinate, alkylamines such as pheniramine, chlorphenamine (chlorpheniramine), dexchlorphenamine, brompheniramine or triprolidine, piperazines such as cyclizine, hydroxyzine, or meclizine and tricyclic H₁-antihistamines such as promethazine, alimemazine (trimeprazine), cyproheptadine, azatadine or loratadine. Further examples of H₁-antihistamines include, but are not limited to dimetindene, acrivastine, astemizole, cetirizine, levocetirizine, loratadine, mizolastine, terfenadine, loratadine, desloratadine, fexofenadine, azelastine, levocabastine and olopatadine.

Examples of H₂-antihistamines include cimetidine, tiotidine, lafutidine, famotidine, and ranitidine. An illustrative example of a H₄-antihistamine, believed to be a H₄-receptor antagonist, is thioperamide.

Leukotrienes are molecules released by blood inflammatory cells in tissues responding to allergic reactions and to inflammatory stimulants. Leukotriene modifying agents (also called anti-leukotrienes) reduce the effect of leukotrienes by interfering with their biosynthesis or the respective receptors. Leukotriene modifying agents have bronchodilatory effects and anti-inflammatory effects. Examples of suitable leukotriene receptor antagonists include, but are not limited to, Montelukast and Zafirlukast. An illustrative example of a leukotriene modifying agent that interferes with leukotriene biosynthesis is the lipoxygenase inhibitor Zileuton.

A mast cell degranulation inhibitor blocks the release of histamine and other mediators from mast cells. Two illustrative examples of a suitable mast cell inhibitor are Cromolyn and Nedocromil.

An illustrative example of a decongestant (supra) is a methylxanthine derivative, such as e.g. caffeine, theophylline, theobromine, aminophylline, doxofylline, pentoxifylline. Two further illustrative examples are ephedrine and pseudoephedrine.

β2-adrenoceptor agonists are strong bronchodilators. They are particularly useful in the treatment of asthma. Examples of β2-adrenoceptor agonists include, but are not limited to terbutaline, orciprenaline, soprenaline, fenoterole, salbutamol, and formoterol.

In some embodiments the pharmaceutical composition further includes an allergen, an immunogenic fragment of an allergen, including a respective immunogenic homolog of each, as further detailed below. A respective allergen may for instance be an insect allergen, a mite allergen (such as a dust mite allergen, including a storage mite allergen), a plant allergen or any other compound causing an allergic reaction in a mammal, including a human. In some embodiments the allergen is cross-reactive with the mite allergen encoded by the heterologous nucleic acid sequence of the recombinant lactobacillus. Typically such a cross-reactive allergen includes at least one common epitope with the at least immunogenic fragment of a mite allergen, or immunogenic homolog thereof, encoded by the respective sequence included in the recombinant lactobacillus, for example expressed by the lactobacillus (cf. also below). As an illustrative example, a number of allergens of other invertebrates are known to be cross-reactive with mite allergens, such as cockroach allergens, silverfish (Lepisma saccharina) and chironomids allergens, shrimp allergens and snail allergens.

The present invention furthermore features a pharmaceutical kit that includes a pharmaceutical composition as described above. In addition to a pharmaceutical composition that includes the recombinant lactobacillus of the invention, the pharmaceutical kit includes at least an immunogenic fragment of an allergen, or an immunogenic homolog thereof. Typically the respective allergen or immunogenic fragment thereof (including a respective immunogenic homolog), as included in the pharmaceutical kit, is included in a pharmaceutical composition. The allergen or allergen fragment (including an immunogenic fragment thereof) and the lactobacillus may be included in the pharmaceutical kit in any combination. They may for example be part of the same pharmaceutical composition or part of two separate pharmaceutical compositions that are included within the same pharmaceutical kit.

The allergen may be part of the pharmaceutical composition or be separately included in the pharmaceutical kit. The pharmaceutical kit may for instance be a multi-part pharmaceutical pack where an allergen or an allergen fragment (including respective immunogenic homologs) is maintained separately from a pharmaceutical composition (which may include the recombinant lactobacillus according to the present invention). It may then be admixed prior to administration or be intended to be administered separately from the pharmaceutical composition. The allergen may be any matter with allergenic properties, typically a protein, a polypeptide or a polysaccharide. Usually the allergen is an allergen involved in allergic disease. The allergic disease may be any form of immune system oversensitivity. The allergic disease may manifest itself in symptoms such as itching, skin rash or hives, eczema, dermatitis (atopic dermatitis or contact dermatitis), drainage from the nose or eyes, sinus pressure, sore throat, wheezing, coughing, shortness of breath, swelling of the mouth, lips, or throat, or digestive problems. It may for instance be a skin allergy or a respiratory allergy such as asthma. The allergic disease may be a response to any specific allergen. In some embodiments the allergic disease is mite allergy, such as for example dust mite allergy, including house dust mite allergy.

In some embodiments the allergen included in the pharmaceutical kit, or in the pharmaceutical composition as described above, is included in a therapeutically effective amount or in a prophylactically effective amount. Similarly to the recombinant lactobacillus, the exact therapeutically, or respectively prophylactically, effective amount of the recombinant lactobacillus will depend on a number of factors as e.g. the type of allergen and as also indicated above. Typically, the amount of the allergen is in the range of about 1-1000 μg. It is understood that in some embodiments the allergen is administered weekly, and in some embodiments it is administered monthly. In further embodiments the allergen is administered weekly at the beginning of treatment, whereafter its administration is gradually reduced to monthly administrations for maintenance. Accordingly, the dosage of the allergen is dependent on the type and mode of delivery. As an illustrative example, for injections a daily dose may be in the range of about 0.1 to about 10 μg. For sublingual or oral delivery, a daily dose may for example be in a range of about 10 to about 100 μg/delivery. The pharmaceutical kit may for example include a pharmaceutical composition, which includes an amount in a range that is suitable to achieve a dose of about 1 μg to about 100 μg of the allergen as an effective amount.

In some embodiments the allergen included in the pharmaceutical kit is a mite allergen. In some embodiments this mite allergen is an allergen of a domestic mite. In some embodiments the nucleic acid sequence encodes a dust mite allergen, such as e.g. a house dust mite allergen or a storage mite allergen. The term “dust mite” as used herein refers to a mite that is present in dust. Accordingly the term “house dust mite” is understood to refer to a mite present in house dust. House dust mites therefore include, but are not limited to, the suborder Astigmata and family Pyroglyphidae. Thirteen mite species have so far been identified in house dust. As already partly indicated above, three of them, Dermatophagoides farinae, Dermatophagoides pteronyssinus, and Euroglyphus maynei, which are all found in temperate climates, are worldwide domestically common and are the major source of mite allergens. An illustrative example of a house dust mite in tropical climates is the storage mite Blomia tropicalis (Family Echymyopodidae). Numerous other storage mites can be found in homes and are a potent source of allergens. Examples of further species are included in, but not limited to, the families Glycyphagidae (Glycyphagus domesticus and Lepidoglyphus destructor), Acaridae (Tyrophagus putrescentiae and Acarus siro), and Chortoglyphidae (Chortoglyphus ancutatus). Lepidoglyphus destructor has for instance been found to be the most important allergen in the dust of farms (hay dust and house dust) on the Swedish island of Gotland. Further examples of mites that can be present in dust in homes include predaceous mites (e.g. Cheyletus) and parasitic pacific spider mites, including 2-spotted spider mites, of plants (Tetranychidae and Tarsonemidae).

In other embodiments the mite may for example be a water mite such as Hydrachnidiae, the ear mite (Otodectes cynotis), the demodex mite (Demodex canis) of dogs, the citrus red mite (Panonychus citri), the house mouse mite (Liponyssoides sanuineus), the tropical rat mite (Ornithonyssus bacoti), the bird mite (Ornithonyssus bursa), the chicken mite (Dermanyssus gallinae), the northern fowl mite (Ornithonyssus sylviarum), the mange mite (Trixacarus caviae), the sheep scab mite (Psoroptes ovis), the straw itch mite (Pyemotes tritici), the bamboo mite (Stigmaeopsis longus), the European red mite (Panonychus ulmi), the wheat curl mite (Aceria tosichella), the brown wheat mite (Petrobia latens), the banks grass mite (Oligonychus pratensis), the strawberry spider mite (Tetranychus turkestani), the clover mite (Bryobia praetiosa Koch), or the Varroa mite (Varroa jacobsoni) of honey bees, to name only a few. A number of these mites can for example also be present in house dust. A mite allergen included in the pharmaceutical kit may also be an allergen, or an immunogenic fragment thereof, of a mite that originates from domestic animals, e.g. cattle, such as sheep, pig and cat. Examples include, but are not limited to, Chorioptes bovis, Psoroptes ovis, Sarcoptes suis and Notoedres cati. Mite allergens are proteins or parts of proteins that originate from a mite, in particular a mite body or mite feces. Group 1, 3, 4, 6, 8 and 9 allergens of Dermatophagoides are for example enzymes, whereas group 10, 11 and 13 allergens are known to be tropomyosins, paramyosins and fatty acid-binding proteins, respectively. Most mite allergens, including dust mite allergens, originate from the digestive tract of a mite and are accordingly found at high levels in mite feces. Typical examples of mite allergens are enzymes originating from the mite's digestive tract. As an illustrative example, the allergen Der p 6 from Dermatophagoides pteronyssinus has been characterized in terms of substrate affinity as mite chymotrypsin. As a further illustrative example, Der p 1 from Dermatophagoides pteronyssinus, is a cysteine protease. A further illustrative example of a mite allergen is an enzyme associated with the molting process that occurs as a mite changes from one life stage to a subsequent one. As an illustrative example, Der p 2 from Dermatophagoides pteronyssinus has been found to be sequentially homologous to esr16, a protein from moths that is expressed coincident with molting. Yet another illustrative example of a mite allergen is a component of mite saliva that is left in the environment on food substrates where mites feed.

Examples of a mite allergen include, but are not limited to, Der p, 1 proper p 1, Der p 2, Der p 3, Der p 4, Der p 5, Der p 7, Der p 8, Der p 9, Der p 10, Der p 11, Der p 14, Der p 15, Der p 18, Der f 1, Der f 2, Der f 3, Der f 4, Der f 5, Der f 6, Der f 7, Der f 10, Der f 11, Der f 15, Der f 16, Der f 18, Der ml, Eur m 1, Eur m 2, Her f2, Blot 1, Blot 3, Blo t 5, Blo t 12, Fel d 1, Mag 1, Mag 3, Tyr p 2, Lep d 1, Lep d 2, Lep d 5, Lep d 7, Lep d 10, and Lep d 13. In some embodiments the mite allergen (including a fragment thereof or respective homolog) included in the pharmaceutical kit includes at least one common epitope (cf. supra) with the mite allergen (including a fragment thereof or respective homolog) expressed by the recombinant lactobacillus. In this respect it should be noted that some allergens are shared by different mite species, while other antigens are unique to a selected mite species. As an example, Dermatophagoides farinae shares several allergens with Dermatophagoides pteronyssinus and Tyrophagus putrescentiae. While any mite allergen may be used throughout the present invention, it may therefore in some embodiments be desired to select an antigen that is shared with e.g. only few or no other mite species than the species of interest. In one embodiment the mite allergen is thus for example a mite allergen that is cross-reactive with the at least immunogenic fragment of an allergen, or immunogenic homolog thereof, expressed by the recombinant lactobacillus. In another embodiment the mite allergen included in the pharmaceutical kit is the mite allergen, mite allergen fragment, or respective homolog expressed by the recombinant lactobacillus.

A respective allergen or fragment thereof, including an immunogenic homolog, may be obtained from any source. It may for example originate from a natural source or have been synthesized. It may for instance be enriched, purified or isolated from a source that is known or suspected to contain the allergen. In case of a dust mite allergen, dust, mite saliva, or mite feces may for instance be collected to enrich, purify or isolate allergens or allergen fragments therefrom. The allergen or fragment may also be enriched, purified or isolated from organisms, tissues or cells that naturally produce the polypeptides. Alternatively, the allergen or fragment thereof can be expressed as a polypeptide in any organism, typically in recombinant or transgenic form. In some embodiments the allergen (including an immunogenic homolog thereof) or immunogenic fragment of the allergen (including an immunogenic homolog thereof), is obtained by any one of enrichment, purification and isolation from a recombinant organism, such as a recombinant microorganism. Where it is desired to obtain an allergen or fragment with posttranslational modifications present in the naturally occurring allergen, the selection of an eukaryotic organism, or a prokaryotic organism expressing the required enzymes for posttranslational modification, may be advantageous. An illustrative example of an enriched allergen is an extract of a respective allergen, for instance supplied as a sterile solution intended for subcutaneous, intracutaneous or sublingual administration. Such allergen extracts are commercially available, for example the “PMG Mite Mix/G33G3805” from Hollister-Stier Laboratories, “Staloral” (for sublingual delivery) from Stallergènes, the “Allergenic Extract Standardized Mite” from Greer or the “RX mix house dust mold inhalant injection” from ALK Abello Inc.

The term “enriched” in reference to a molecule such as an allergen or allergen fragment means that the specific molecules constitutes a significantly higher fraction (such as 2-5 fold) of all molecules present in the cells or solution of interest than in normal or diseased cells or in the cells from which the molecule was taken. The term significant here is used to indicate that the level of increase is useful to the person making such an increase, and generally means an increase relative to other matter, e.g. amino acid sequences, of about at least 2-fold, such as at least 5- to 10-fold or even more. The term also does not exclude the presence of allergens from other sources. Such other source of an allergen may, for example, include an allergen from the environment or encoded by a yeast or bacterial genome, or a cloning vector. It is understood that the term is meant to cover only those situations in which man has intervened to increase the proportion of the desired matter, e.g. the desired allergen.

An enrichment may for instance include obtaining a fraction from a cell extract, such as for instance a nuclear fraction, a plasmamembrane fraction, or a microsome fraction. This may be obtained by standard techniques such as centrifugation. Examples of other means of enrichment are filtration or dialysis, which may for instance be directed at the removal of molecules below a certain molecular weight, or a precipitation using organic solvents or ammonium sulphate. The term “purified” in reference to matter, such as an allergen, is understood to be a relative indication in comparison to the original environment of the matter, thereby representing an indication that e.g. the allergen or allergen fragment is relatively purer than in the natural environment. It does therefore not refer to an absolute value in the sense of absolute purity (such as a homogeneous preparation). Compared to the natural level the level of a purified allergen or allergen fragment should be at least 2-5 fold greater (e.g., in terms of μg/ml). Purification of at least one order of magnitude, such as two or three orders of magnitude, is expressly contemplated. The purified allergen or allergen fragment—or the immunogenic homolog thereof—is typically essentially free of contaminating matter that shows an overlapping or similar immunogenic activity (such as a so called cross-reaction), for example 90%, 95%, or 99% pure.

A purification may for instance include a chromatographic technique, for example gel filtration, ion exchange chromatography, affinity purification, hydrophobic interaction chromatography or hydrophobic charge induction chromatography. A purification may also include a combination or a plurality of such techniques and other methods. Another illustrative example of a purification is an electrophoretic technique, such as preparative capillary electrophoresis. An isolation may include the combination of similar methods. The term “isolated” indicates that naturally occurring matter or a naturally occurring sequence has been removed from its normal cellular (e.g. intracellular) environment. Thus, the matter or sequence may be in a cell-free solution or suspension etc., or placed in a different cellular environment. The term does not imply that the matter or sequence is the only the matter or sequence present, but that it is essentially free (usually about 90-95% pure at least) of other matter naturally associated with it.

The allergen or allergen fragment can be isolated from a natural source by methods well known in the art. The natural source may for example be mammalian—for instance human—blood, semen, or tissue. In some embodiments the allergen or allergen fragment may be obtained from an organism or cell that has been altered to express the polypeptide. By means of technologies of genetic manipulation well established in the art a cell or organism can be made to produce a protein which it normally does not produce or which the cell normally produces at lower levels. An illustrative example is a recombinant eukaryotic or prokaryotic host cell or a transgenic organism.

In another embodiment the polypeptide may be synthesized in vitro by e.g. chemical or enzymatic methods starting from amino acids. Chemical methods are well known in the art and involve sequential steps of protection, activation, coupling and selective deprotection of reactive functional groups of the amino acids involved, as well as of the growing peptide chain. Enzymatic strategies involve for example the blockwise coupling of separately produced synthetic fragments by proteases. Where desired, a commercially available automated polypeptide synthesizer may be used.

The allergen (including a fragment thereof or respective homolog) may be provided with a suitable carrier and/or diluent as indicated above. The allergen may for example be chemical coupled to an appropriate carrier protein. As a further example, it may be incorporated into liposomes, or conjugated to polysaccharides and/or other polymers for use in a vaccine formulation.

The present invention furthermore provides a method of modulating, including controlling, the immune response to an allergen in a mammal. The modulation of an immune response to an allergen in a mammal is typically carried out in order to relieve or alleviate fully or at least to some extent one or more of the symptoms of an allergic condition, or to prevent fully or to at least some extent one or more of the symptoms of an allergic condition. The mammal may for instance be a mouse, a rat, a rabbit, a hamster, a dog, a cat, a marmoset, an ape, or a human. In typical embodiments the method of the invention is, or is included in, a method of treating an allergic disease. As used herein, the terms “treating” and “treatment” include alleviating, substantially inhibiting, reducing, slowing, eliminating or reversing the progression of an allergic condition, at least substantially, or to a certain degree, ameliorating clinical or aesthetical symptoms of an allergic condition, at least substantially, or to a certain degree, preventing the appearance of clinical or aesthetical symptoms of such a condition, or preventing or delaying, at least to a certain degree, the reoccurrance of the condition in a previously afflicted subject.

The method includes administering a composition as described above. In this regard, the present invention also relates to the use of a recombinant lactobacillus as described above in the manufacture of a pharmaceutical kit for modulating, including controlling, the immune response to an allergen. In this respect the present invention likewise relates to the use of the respective lactobacillus in modulating the immune response to an allergen. The use as well as the method may for example be in the treatment or prophylaxis of allergy, for example mite allergy, such as dust mite allergy, for instance house dust mite allergy. The allergen is typically a mite allergen, for instance a dust mite allergen (cf. above for examples), such as e.g. a house dust mite allergen. In some embodiments the mite allergen, for modulating the response to which the respective lactobacillus is used, includes at least one common epitope (cf. above) with the mite allergen expressed by the recombinant lactobacillus. In one embodiment the mite allergen is the mite allergen expressed by the recombinant lactobacillus. Examples of a respective allergy include (cf. also above for examples of symptoms), but are not limited to, asthma, rhinitis (hay fever), atopic dermatitis (eczema) and urticaria (hives). A respective allergy may also be associated with symptoms such as coughing, sneezing, nasal congestion, sore throat, postnasal drip, flushing, and nausea.

The recombinant lactobacillus may be administered to the host by any means, as long as the lactobacillus can be used for modulating the immune response to an allergen. Likewise, the entire pharmaceutical kit may be administered by any means. Typically the recombinant lactobacillus is included in a pharmaceutical composition when administered to a mammal. The same generally applies to an antigen if included in the pharmaceutical kit. It should generally also be desired to use a form of administration that is not unnecessarily harmful to the host. The skilled artisan will thus appreciate that the present invention allows for instance oral or sublingual administration of the recombinant lactobacillus. In some embodiments the entire pharmaceutical kit may be administered orally or sublingually.

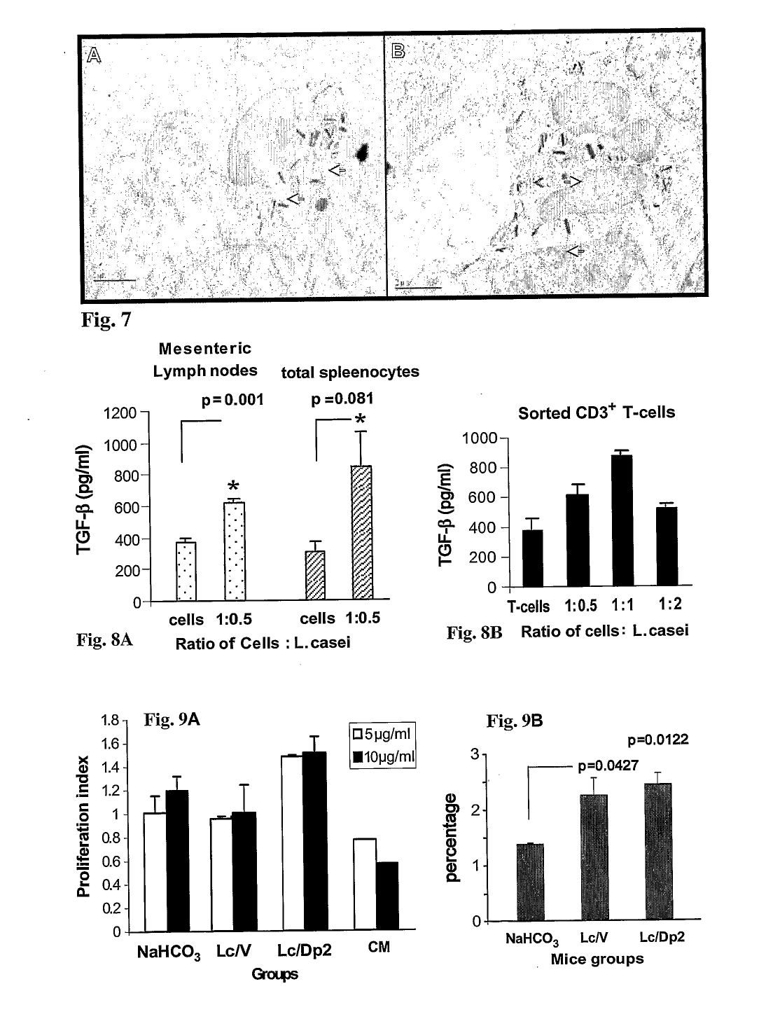

In order to be able to track orally administered lactobacillus it may in some embodiments be desired to mark the lactobacillus or to mark a corresponding lactobacillus administered concomitantly. An illustrative example is the use of enhanced green fluorescence protein (eGFP) in the same vector as the allergen, whether-together with the allergen or on a separate vector instead thereof. FIG. 6 illustrates the expression of eGFP in pL500 vector in L. casei Shirota and the monitoring of orally administered live recombinant lactobacilli in the gastrointestinal tract of mice. The example illustrates that live recombinant L. casei Shirota-eGFP when orally administered to mice is able to translocate into both the T and B-cell regions of the intestinal Peyer's Patches as determined by confocal microscopy (cf. FIG. 6). This is further confirmed by transmission electron microscopy showing intact L. casei Shirota-eGFP in the vacoules of mono- and polymorphic cells in the Peyer's patches (cf. FIG. 7).

The use of a lactobacillus for a prophylactic or therapeutic purposes (cf. also below) has several advantages. Firstly the traditional use of lactobacilli in dairy foods is of the “Generally Recognized as Safe” (GRAS) status. They are used in specified standardized foods such as yogurt, cheese and buttermilk. The pathogenic potential of lactobacilli is very low. Lactobacilli are also probiotic bacteriae in that they eg. prevent intestinal infections, reduce serum cholesterol levels and show anti-carcinogenic activity. The term “probiotic” refers to a living or inactivated organism with beneficial effects on health when ingested. Secondly, different lactobacillus strains are currently being used in consumer food kits (eg. infant milk powder) and as such the recombinant lactobacilli can be developed into a stable and convenient food-grade kit. Consumption of for instance a food grade vaccine kit is convenient and highly compliant when compared to the parenteral route (which is invasive and painful), in addition to the possibility of multiple-doses and large scale/herd immunization which are economical and important in less industrialized countries. Thirdly, although lactobacilli have low intrinsic immunogenicity, the cell wall components of a lactobacillus (eg. peptidoglycan) are capable of not only conferring adjuvant properties on any foreign antigen/allergen expressed or coupled to the bacteria but also an immuno-modulatory effect on immune responses. The delivery of antigens/allergens to mucosal-associated lymphoid tissues in paediatric and immuno-compromised populations by safe, non-invasive means, such as lactobacilli, represents a crucial improvement to prevailing vaccination options.

In some embodiments the method includes repeatedly administering a respective lactobacillus, whether in a pharmaceutical composition or a pharmaceutical kit for repeated administration of the same, or a pharmaceutical composition included therein respectively. Correspondingly, in some embodiments the recombinant lactobacillus is used in a pharmaceutical composition or a pharmaceutical kit for repeated administration of the respective lactobacillus. In some embodiments the entire pharmaceutical composition or pharmaceutical kit is for repeated administration.

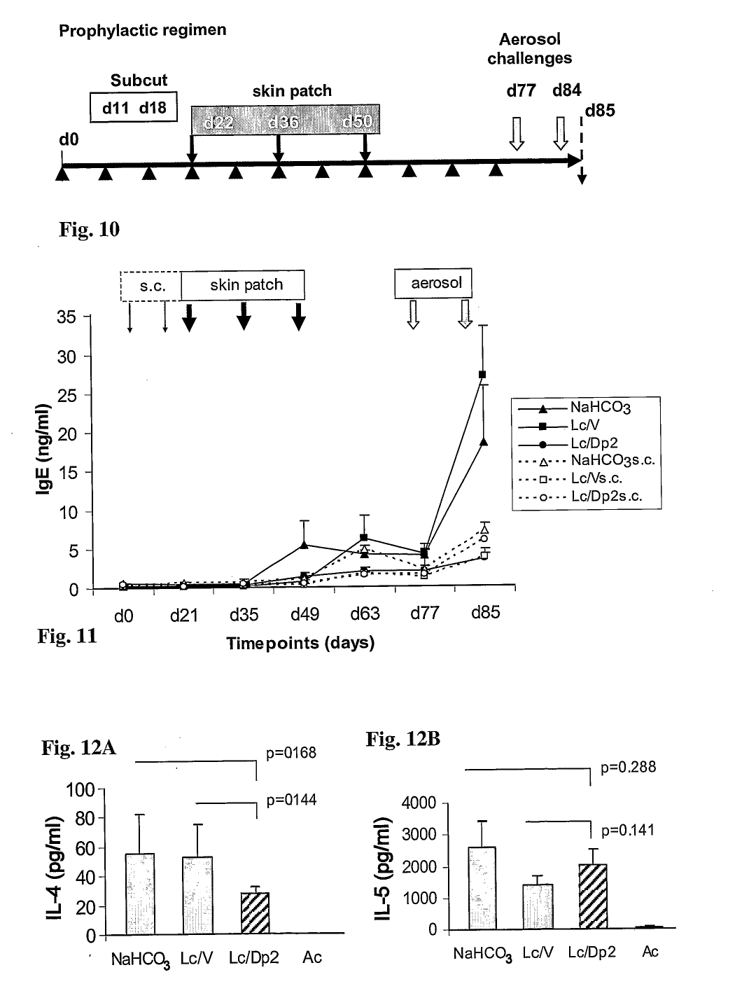

The effective control or modulation of the immune response to an allergen can be monitored by any means known in the art. As an illustrative example, the levels of factors, e.g. polypeptides or proteins, involved in immune responses, in particular those involved in allergic immune responses can be monitored. The appending figures and examples illustrate ways of respective monitoring. FIG. 8 for instance shows that in-vitro co-cultured T-cells from spleen and mesenteric lymph nodes of naïve mice with L. casei Shirota mediate secretion of TGF-β, a regulatory T-cell cytokine. A Der p 2-specific T-cell priming (cf. below) is shown by the Der p 2-specific proliferation of cells from Peyer's patches of mice fed with the recombinant L. casei Shirota/Der p 2 (Lc/Dp2), and not NaHCO₃ (cf. FIG. 9). The respective figure further shows that mice orally administered with either L. casei/pLP500 (Lc/V) or Lc/Dp2 for four consecutive days show an increase in the subset of CD3⁺CD4⁺D25⁺ T-cells in the mesenteric lymph nodes (MLNs) compared to mice fed with NaHCO₃ (FIG. 9B). This indicates an induction of a group of Tr cells in mice fed with probiotics L. casei.

The method of the present invention further features the combined administration of any naturally occurring lactobacillus and an allergen (cf. above for examples). In some embodiments the lactobacillus is selected from the groups consisting of Lactobacillus casei, Lactobacillus acidophilus, Lactobacillus fermentum, Lactobacillus gasseri, Lactobacillus pentosus, Lactobacillus plantarum, Lactobacillus sporogenes, Lactobacillus brevis, Lactobacillus delbrueckii, Lactobacillus salivarius, Lactobacillus hilgardii, Lactobacillus lactis, Lactobacillus rhamnosus, Lactobacillus johnsonii, Lactobacillus leishmanis, Lactobacillus jensenii, Lactobacillus reuteri, Lactobacillus sakei, Lactobacillus cellobiosus, Lactobacillus crispatus, Lactobacillus curvatus, Lactobacillus caucasicus, and Lactobacillus helveticus. In some embodiments the naturally occurring lactobacillus is furthermore administered in combination with the recombinant lactobacillus of the invention. In some of these embodiments the naturally occurring lactobacillus is of the same species as the recombinant lactobacillus. In other embodiments the naturally occurring lactobacillus is administered in combination with another bacterium, for example another probiotic bacterium such as for instance Streptococcus faecalis, Clostridium butyricum or Bacillus mesentericus. In some embodiments a respective lactobacillus may be included in the same pharmaceutical composition as the respective allergen. In other embodiments the lactobacillus and the allergen may be provided separately. They may for instance be included in separate pharmaceutical compositions. Such pharmaceutical compositions may be included in a common pharmaceutical kit.

In some embodiments the method of the invention includes a combined administration (cf. below) of the recombinant lactobacillus of the invention with an allergen or an immunogenic homolog of an allergen. In some embodiments the recombinant lactobacillus may be administered in combination with an immunogenic fragment of an allergen, or an immunogenic homolog thereof. This mite allergen, immunogenic fragment thereof or respective immunogenic homolog may for example be obtained by any one of enrichment, purification and isolation from a recombinant organism, as explained above. In some embodiments it may thus be administered together, for instance concomitantly, with an allergen or a homolog thereof. As an illustrative example, the lactobacillus may be used in the manufacture of a pharmaceutical kit that also includes an allergen. The lactobacillus may thus be part of a pharmaceutical kit together with an allergen. The lactobacillus and the allergen may for instance be part of a pharmaceutical composition included in the pharmaceutical kit. In other embodiments the lactobacillus may be part of a pharmaceutical composition, while the allergen is a separate component or included in a separate component, such as a further pharmaceutical composition, of the pharmaceutical kit.

The respective allergen, allergen fragment or respective homolog that is included in the pharmaceutical kit or in the pharmaceutical composition that includes the lactobacillus is typically a mite allergen, for instance a dust mite allergen (cf. above for examples), such as e.g. a house dust mite allergen. In some embodiments the mite allergen, for modulating the response to which the respective lactobacillus is used, includes at least one common epitope (cf. above) with the mite allergen, mite allergen fragment, or respective homolog expressed by the recombinant lactobacillus. The mite allergen (including a fragment thereof or respective homolog) may for instance be cross-reactive to the allergen (including a fragment thereof or respective homolog) expressed by the lactobacillus. In one embodiment the mite allergen, fragment thereof, or respective homolog, is the mite allergen, fragment thereof, or respective homolog, expressed by the recombinant lactobacillus. The application of the allergen may be carried out by any route and method. The allergen (including a fragment thereof or respective homolog) may for instance be applied sublingually, subcutaneously, intradermally, transdermally, epicutaneously or any combination thereof. As an illustrative example, the allergen or allergen fragment, or an immunogenic homolog thereof (e.g. included in a pharmaceutical composition) may be administered subcutaneously, and the lactobacillus (e.g. included in a pharmaceutical composition) may be administered orally. Like the lactobacillus, the allergen or allergen fragment may be applied once or several times, for instance at selected time intervals. In some embodiments it may thus be administered repeatedly.

The skilled artisan will appreciate not only the advantage of using a lactobacillus (supra), but also the surprising finding of the present inventors that a combined application of the lactobacillus and a respective allergen results in a synergistic effect for the therapy and prevention of allergy, which is also illustrated in the examples below. In one aspect the present invention therefore also provides a double-modality approach for effective therapeutic and prophylactic strategies for allergy.

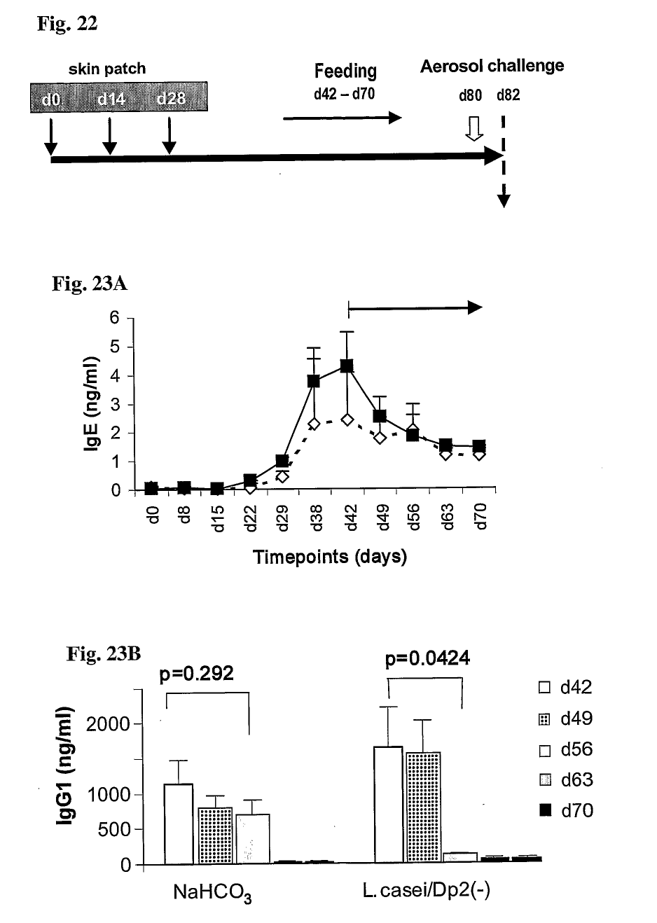

The lactobacillus and the allergen, allergen fragment or respective homolog may be administered independently from each other in an independent dosage. Accordingly, any number of applications of the lactobacillus and the allergen (including a homolog) or fragment thereof may for example be carried out simultaneously or consecutively over time. The lactobacillus may thus e.g. be used as an adjuvant that can modulate, e.g. enhance, the immune response when given at the same time as the allergen. In some embodiments the lactobacillus and the allergen, allergen fragment or respective homolog are used sequentially. During a selected time interval in e.g. a dose regimen only the allergen (including a fragment thereof or respective homolog), only the lactobacillus or only the allegen may be administered. As an illustrative example, where the lactobacillus is administered several times, the allergen may be administered in advance, between two applications of the lactobacillus or after terminating applications of the lactobacillus, and vice versa. In some embodiments the lactobacillus is administered repeatedly, e.g. once a day. The allergen is then administered after one or more applications of the lactobacillus over a period of time. Thereafter only the lactobacillus is further repeatedly administered. It is understood that in such a case the form of administration of the lactobacillus and/or the allergen may change, as for instance depicted in FIG. 10 or FIG. 16. In some embodiments the lactobacillus may be applied first as e.g. described below. In other embodiments the allergen may be applied first, as e.g. shown in FIG. 22.

Accordingly the administration of the allergen (including a fragment thereof or respective homolog) and the lactobacillus may be carried out in form of one or more independent individual doses, such as a so called “prime boost” regimen or method in a prophylactic or therapeutic manner. In such a regimen (or method) the lactobacillus may for example be delivered in a “priming” step and, subsequently the allergen may be delivered in a “boosting” step, or vice versa. Where e.g. the lactobacillus is administered first it may be termed the “primer”, the subsequently administered allergen may be called the “booster”. Where e.g. the allergen is administered first it may be termed the “primer”, the subsequently administered lactobacillus may then be called the “booster”. It is understood that the terms “priming” and “boosting” refer to the effect of the antigen and the lactobacillus on the host organism rather than the order in which they are being administered. Therefore, the primer may be administered before, at the same time or after the booster. An administration after the boosting composition may for instance be desired if the boosting composition is expected to take longer to act.

Such a “prime boost” regimen may for instance be used for prophylaxis, i.e. to reduce, diminish or prevent the immune response to an allergen in advance. In such a case it may also be desired to reduce or prevent the effect of an oversensitivity of the immune system of a host to a corresponding allergen. In such embodiments “priming” may also refer to a method whereby a first administration (e.g. of the antigen) is an immunisation that permits the generation of an immune response upon a second administration with the same antigen (e.g. of the lactobacillus), wherein the second immune response is greater than that achieved where the first immunization is not provided. In some embodiments a respective prophylactic regimen may be used to protect an animal or an individual against allergen sensitization.

In some embodiments the method of controlling the immune response to an allergen in a mammal (e.g. a human being) includes:

(a) providing a recombinant lactobacillus according to the invention or a pharmaceutical composition that includes a recombinant lactobacillus as described above,

(b) administering the recombinant lactobacillus according to the invention or the pharmaceutical composition that includes a recombinant lactobacillus,

(c) providing at least an immunogenic fragment of an allergen (or a respective immunogenic homolog thereof) or a pharmaceutical composition that includes at least an immunogenic fragment of an allergen (or a respective immunogenic homolog thereof) as described above, and

(d) administering the at least immunogenic fragment of an allergen (or a respective immunogenic homolog thereof) or the pharmaceutical composition that includes at least an immunogenic fragment of an allergen (or a respective immunogenic homolog thereof).

As an illustrative example, the method may include the following steps:

(a) priming of a mammal with a therapeutically effective amount of a recombinant lactobacillus according to the invention, or a pharmaceutical composition according to the invention,

b) optionally repeating step a) between one and three times after between a day and about a week;

c) boosting of the animal with a therapeutically effective amount of an allergen; and

d) optionally repeating step c) between one and five times after subsequent time periods of between about a week to about a month.

As indicated above, the allergen may in some embodiments be the allergen or correspond to the allergen expressed by the recombinant lactobacillus according to the invention.

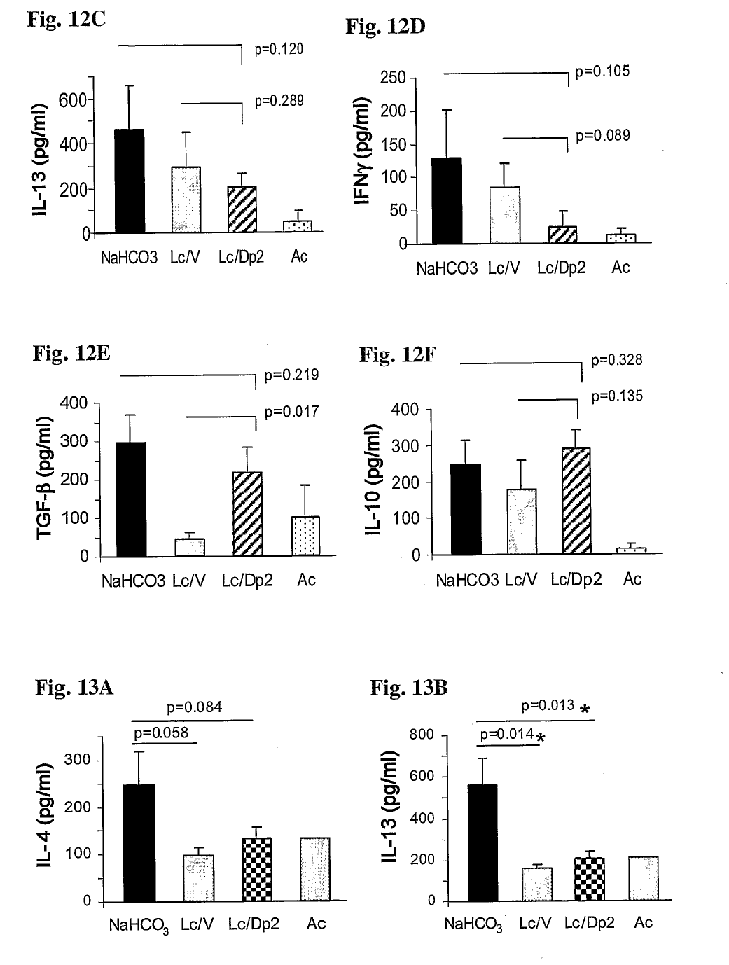

Accordingly, in some embodiments the administration of the lactobacillus serves in priming, while the administration of the antigen (including a fragment thereof or respective homolog) serves in boosting. A respective example is depicted in FIG. 10. In this example orally administered recombinant L. casei Shirota expressing Der p 2 was used in combination with s.c. boosting of Der p 2 in a prophylactic regimen. A comparison to the use of ineffective NaHCO₃ instead of lactobacillus shows that administration of recombinant L. casei Shirota expressing Der p2 alone can suppress IgE generation even after airways challenge (cf. FIG. 11). In contrast thereto, the level of Der p 2-specific serum IgG1 was not affected. Likewise, generation of Th-2 cytokines and pro-inflammatory cytokines by T-lymphocytes was downregulated (FIG. 12).

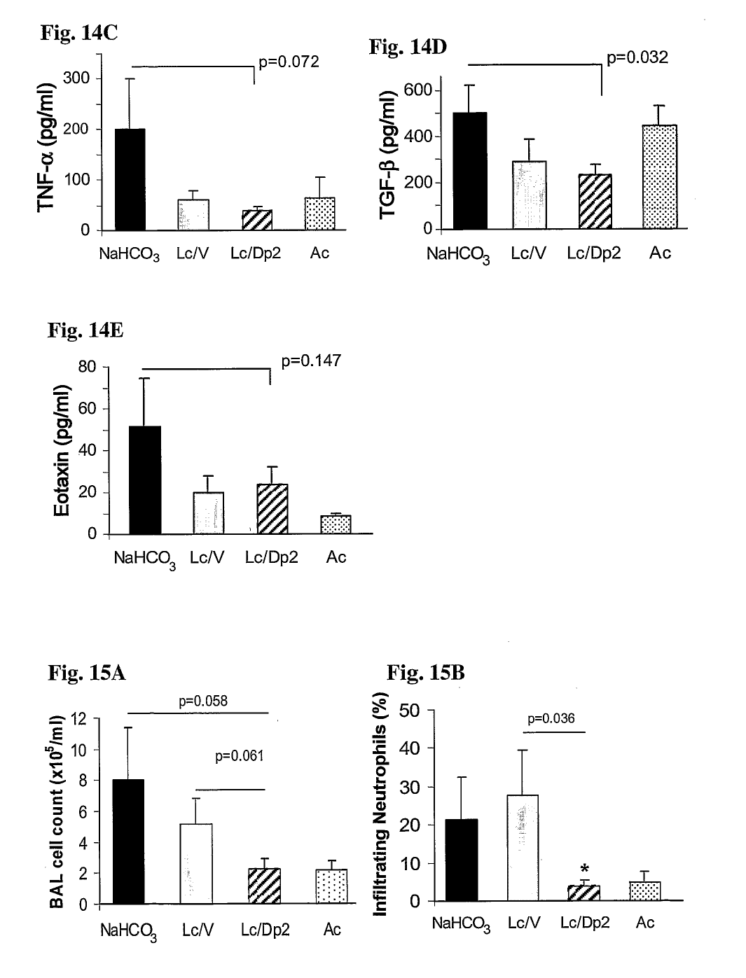

In this example, administration of recombinant L. casei Shirota expressing Der p 2 primed for a mixture of Th-2 and Der p 2-specific Tr cells (cf. also below). These Der p 2-specific Tr cells may be capable of exerting an inhibitory or tolerogenic effect on existing Th-2 cells via regulatory cytokines such as TGF-β1. It was furthermore observed that circulating interleukin-10 (IL-10) cytokine levels in sera of mice that had been administered recombinant L. casei Shirota expressing Der p 2 obtained after airways challenge are significantly reduced compared to control groups. IL-10 is a pleiotropic cytokine, generated by Th2 cells or T regulatory-type lymphocytes. In atopic allergy and asthma an increased expression of interleukin-10 has previously been observed. IL-10 produced by T regulatory cells is anti-inflamatory and capable of modulating Th1-type cytokine and/or Th2 production. Th-2 cytokines are further decreased. Levels of eotaxin were also reduced. Eotaxin is an important chemokine modulating allergic inflammation and serum concentration of eotaxin have previously been found to be elevated in e.g. asthma. A significant reduction in levels of transforming growth factor-β1 (TGF-β1) was also observed (cf. FIG. 14). TGF-β1, a commonly known cytokine for Tr cells survival and function, is generated by eosinophils in the lung and is known to regulate Th-2 cytokine-induced eotaxin release. It also serves as a growth factor for fibroblasts, thus an increase of TGF-β1 in mucosal associated lymphoid tissues of the lung has been reported to augment or exacerbate airway remodeling.

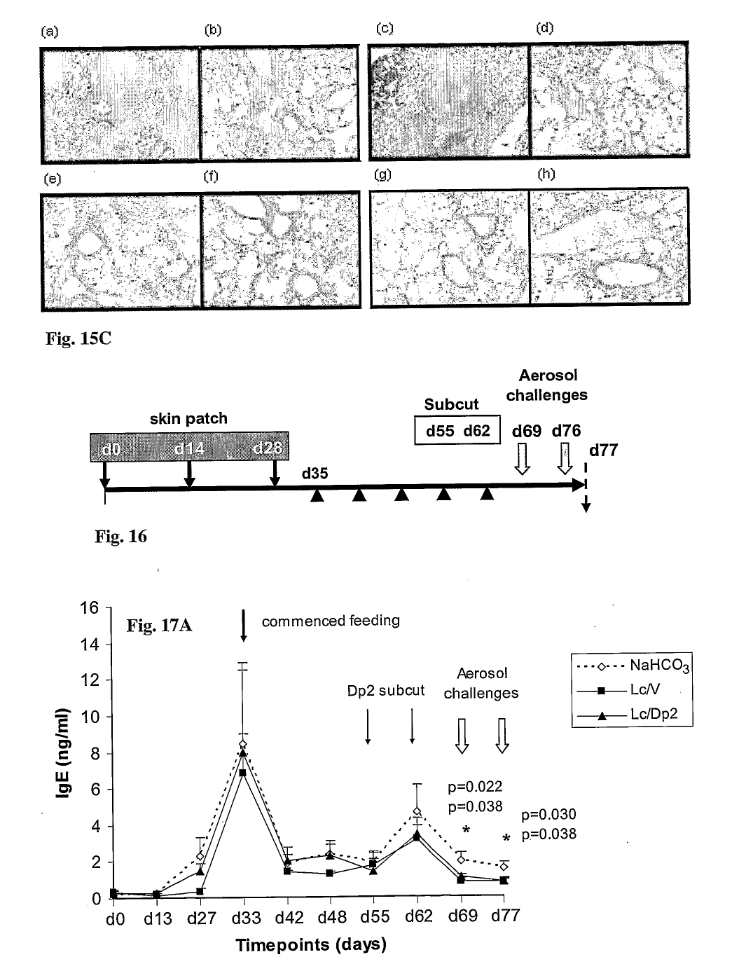

Concurrently, these mice also show reduced cell numbers in the broncholalveolar lavage fluid (BALF) (FIG. 15A), with level similar to that of the Ac group, i.e. the group of mice only treated with the respective allergen and an aerosol challenge. The number of neutrophils, which are part of the inflammatory response to an antigen and which are known to be activated via an IgE receptor, was also significantly reduced in mice that had been administered recombinant L. casei Shirota expressing Der p 2 (FIG. 15B). The number of eosinophils, which is known to increase in allergic diseases, was low. Histological analysis of lung sections obtained 24 h after airways challenge showed different degree (moderate to servere) of airway pathology in mice that had been administered with a negative control (NaHCO₃ or lactobacilli with a vector not encoding a mite allergen). Furthermore inflammatory infiltrates surrounded the bronchoalveolar spaces of these mice. In contrast thereto, lung sections of mice that had been administered recombinant L. casei Shirota expressing Der p 2 showed substantial reduction in lung inflammation and had a profile similar to that of Ac mice (cf. FIG. 15C). As can be inferred from this example, priming with a recombinant lactobacillus in combination boosting with an allergen is effective in down-regulating an allergic response.

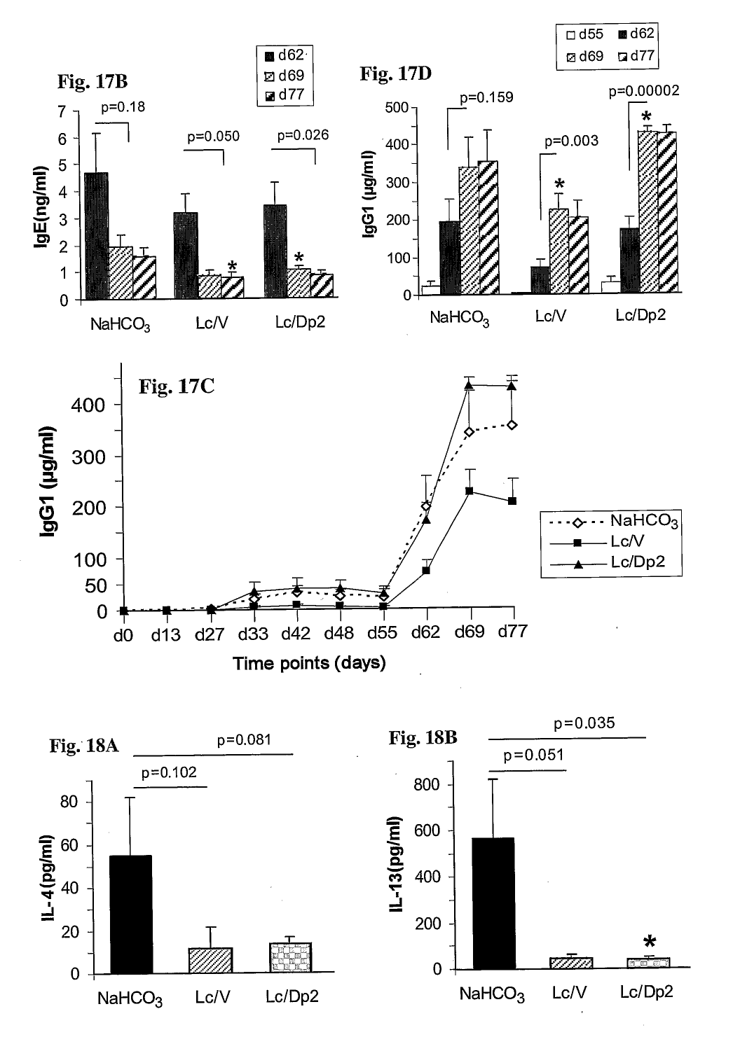

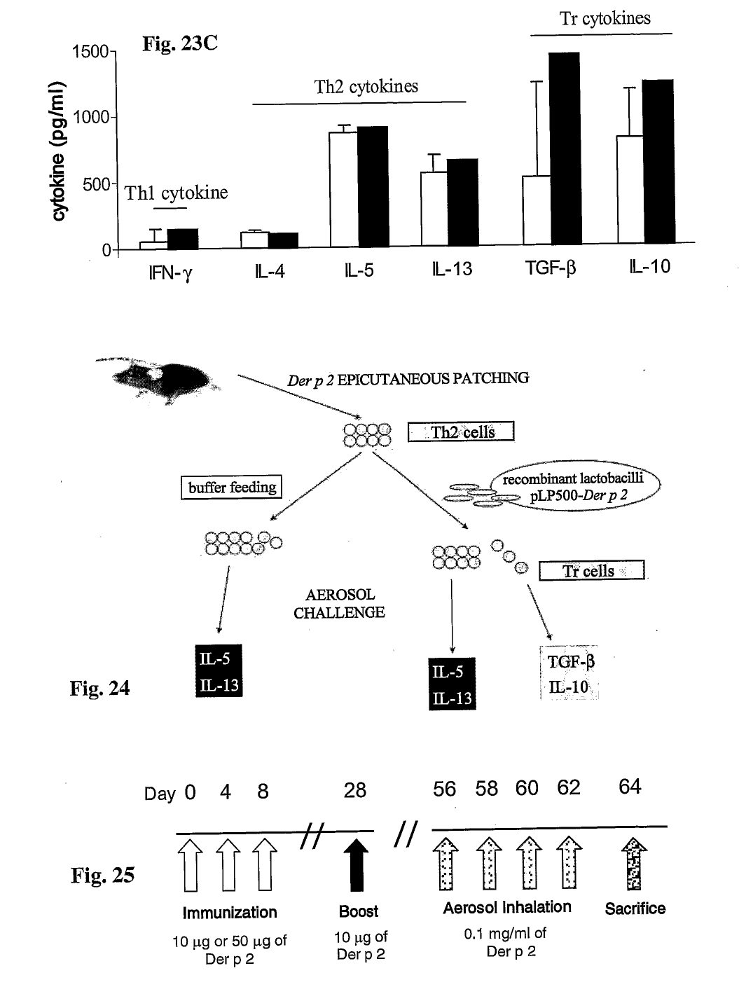

As a further example, the treatment or prophylaxis of an allergic immune response to an allergen may be immunotherapy. A combination of immunization with an allergen in immunotherapy may for instance be combined with the administration of the lactobacillus for therapeutic purposes (cf. below in the appending examples). Such a combination can substantially improve the efficacy and also shorten the duration required for immunotherapy. Serum levels of antigen specific IgE are for instance significantly reduced (cf. FIG. 17.A and FIG. 23A). In order to achieve such effects based on a conventional immunotherapy using the respective antigen alone, high doses of antigen are needed, as depicted in e.g. FIG. 25 and FIG. 26. Only subcutaneous doses of 50 μg antigen (i.e. a high dose) attenuated IgE levels, while doses of 10 μg antigen, i.e. a low dose, caused an increase in IgE levels (cf. FIG. 26A). Therefore the combination of the lactobacillus and the respective antigen reduces the inconvenience of conventional immunotherapy, reduces the frequency and duration of treatment therein, and is likely to reduce the risk of anaphylaxis and to improve efficacy. The potential and efficacy of a recombinant lactobacillus according to the present invention in a prime-boost strategy for the development of a food-based intervention strategy for both prophylaxis and therapy of allergic diseases is also demonstrated by the appending examples.

As will be apparent from the above, the use of a recombinant lactobacillus according to the present invention is advantageous compared to current methods both for allergy therapy and for allergy prevention. Furthermore, a combined application of a respective lactobacillus and an antigen, for instance in immunotherapy, greatly enhances the efficacy of recombinant allergen based immunotherapy. A respective double-modality approach generates the synergistic effect for the effective treatment of allergic diseases. In this regard, allergy prevention by vaccination can likewise be carried out by a combined application of a recombinant lactobacillus of the present invention and an allergen. The recombinant lactobacillus expressing an allergen or a fragment thereof can for instance be used as a food-based antigen-specific prophylactic vaccine to prime the immune system followed by the boosting effect from the administration of an allergen protein. Again, this double-modality approach yields synergistic effects for the prevention of subsequent allergic sensitization.

In order that the invention may be readily understood and put into practical effect, particular embodiments will now be described by way of the following exemplary embodiments and non-limiting examples.

### EXEMPLARY EMBODIMENTS OF THE INVENTION

Exemplary embodiments of a recombinant lactobacillus of the invention, a pharmaceutical kit of the invention and their use are shown in the appended figures.

FIG. 1A depicts schematically a conventional lactobacillus/E. coli shuttle vector, pL500 used for expression of a mite allergen (HDM) gene. Sites for restriction enzymes are indicated. The segment defined by the BamHI and the Nhe I sites was replaced by a sequence encoding the Der p 2 antigen (cf. below). “ldh” indicates lactate dehydrogenase, “Pldh” indicates the promoter sequences of the ldh gene of L. casei. “Tcbh” indicates a transcription terminator sequence of the cbh gene of L. plantarum.

FIG. 1B depicts schematically the intermediate pTUAT vector used for generation of the Der p2/mite allergen expression construct in lactobacilli. Sites for restriction enzymes are indicated.

FIG. 1C depicts schematically a pLP500-HDM expression construct under the control of a constitutive lactate dehydrogenase (ldh) promoter. “Pldh” indicates the promoter sequences of the ldh gene of L. casei. “Anchor” indicates a segment encoding a peptide of 117 amino acids, which is the anchor sequence of L. casei.

FIG. 2 depicts schematically another conventional lactobacillus/E. coli shuttle vector, pL400, likewise used for expression of a mite allergen (HDM) gene. Sites for restriction enzymes are indicated. The segment defined by the BamHI and the Nhe I sites (underlined) was replaced by a sequence encoding the Blo t 5 antigen (cf. below).

FIG. 3 depicts schematically the lactobacillus expression vector pSIP308, a further example of a vector that may be included in a recombinant lactobacillus of the present invention. The reporter gene may be used to include a sequence encoding a mite allergen or a fragment thereof by means of respective restriction enzymes.

FIG. 4 depicts schematically the lactobacillus expression vector pSIP412, yet another example of a vector that may be included in a recombinant lactobacillus of the present invention. A sequence encoding a mite allergen or a fragment thereof may again be introduced at the site of the reporter gene.

FIG. 5 shows the detection of heterologous expression of Der p2 in two strains of lactobacilli, L. casei Shirota and L. rhamnosus gg, by means of Western immunoblot. A total of 3×10⁹ cells was lysed in 1 mL of lysis buffer by sonication. The cell lysate (10 μL) was separated on a 10% Tris-tricine SDS-PAGE gel and subjected to Western immuno-blot assay. A monoclonal immunoglobulin directed against Der p 2 (dilution 1:10,000), biotinylated anti-mouse immunoglobulin (dilution 1:10,000), and peroxidase conjugated—ExtrAvidin (Sigma, dilution 1:5,000) were used. Signals were developed in Superignal® West Pico Chemiluminescent Substrate. Lanes are: “M”: Pre-stain/biotin protein molecular weight marker (BioRad); (1) total lysates from L.gg/pL500, (2) total lysates from L.gg/Der p 2, (3) total lysates from Lcasei/pL500, (4) total lysates from L. casei/Der p 2, and lane (5) recombinant Der p 2 protein produced in yeast (200 ng). The arrow indicates the position of the Der p 2-anchor fusion-protein. It is present in recombinant L. casei Shirota and L. rhamnosus gg, but not in the wildtype used as a control.

FIG. 6 illustrates how L. casei Shirota-eGFP translocates into both T- and B-cell regions of Peyer's patches. The photos depict the view under confocal microscopy. Shown are (A) life L. casei Shirota-eGFP and (B) cryostat sections of Peyer's patches from mice fed with L. casei Shirota-eGFP for four consecutive days. The left shows the location of L. casei Shirota-eGFP bacteria, in (B) respectively in the Peyer's patch section. Immunohistochemically staining with a combination of APC-conjugated anti-mouse THY-1.2 and PE-conjugated anti-mouse CD-19 (staining for T-cell and B-cell regions respectively) of the same field, superimposing all three colours, is depicted on the right, which indicates the location of bacteria in the T-cells and B-cells zone. Confocal microscopy analysis indicated L. casei Shirota-eGFP (cf. left picture) distribution in the T-cell and B-cell region (right), in particular at the regions junction.

FIG. 7 shows the translocation of intact L. casei Shirota-eGFP into the vacoules of mono-(A) and polymorphic (B) cells in Peyer's Patches by transmission electron microscopy. The arrows point to the position of L. casei Shirota-eGFP.

FIG. 8 shows the induction of TGF-β production in T-cells co-cultured in-vitro with L. casei Shirota. (A) Total mesenteric lymph node (MLNs) cells or spleenocytes from naïve mice co-cultured with L. casei at a ratio of 1:0.5 showed a significant increase in TGF-β cytokine production compared to control of cells alone. (B) Sorted CD3⁺ T-cells from spleen of naïve mice co-culture with L. casei at a ration of 1:0.5 or 1:1 also induced an up-regulation of TGF-β production.

FIG. 9 depicts the increase in Der p2-specific T-cells proliferation and regulatory CD4⁺CD25⁺ T-cells in mice fed with recombinant Lc/Dp2. (A) C56BL/6 mice were orally administered with 1×10⁹ cfu/mouse of recombinant Lc/Dp2 or wildtype L.c/V or NaHCO₃ control for one month (three consecutive days per week). Mice were sacrificed at the end of feeding and T-cells isolated from Peyer's patches for proliferation assay using H³-labelled Thymidine incorporation in the absence or presence of 5 μg/ml or 10 μg/ml recombinant yeast Der p2. (B) FACS analysis of cells from mesenteric lymph nodes of mice fed with Lc/Dp2 (n=5), wildtype Lc/V (n=5) or NaHCO₃ (n=6). As shown, the subset of CD4⁺D25⁺ cells in the mesenteric lymph nodes were significantly increase in both Lc/Dp2 and Lc/V group compare that in NaHCO₃ group.

FIG. 10 depicts a prophylactic regimen used. Three group of C57BL/6 mice were established, consisting of C57BL/6 mice (n=5 per group) fed with either NaHCO₃ buffer, heat-killed Lc/V or Lc/Dp2 throughout the experiment. Feedings are indicated by the filled triangles “▴” below the time line. Feeding was carried out with one dose per day for three consecutive days in a week. At d11 and d18, all mice were boosted by subcutaneous immunization of recombinant Der p2 (50 μg/mouse). Subsequently, all mice were sensitized three times by epicutaneous patching (at day 22, 36 and 50) and challenged twice by aerosol with Der p2. Approximately 24 h after the last challenge, mice were sacrificed and broncholalveolar lavage fluid (BALF) obtained for differential cell count and cytokine analysis and the lung for haematoxylin & eosin (H&E) staining. In addition, cells from mesenteric lymph nodes (MLN) and spleen were obtained for primary and secondary T-cell culture and cytokine analyses.

FIG. 11 shows Der p2-specific immunoglobulin responses. The kinetics of Der p2-specific serum IgE for mice receiving s.c. boost with Der p2 showed no significant difference for the three groups whereas mice that did not receive s.c. boost but fed with Lc/Dp2 exhibited significant attenuation of IgE compared to the NaHCO₃ (p=0.042) and Lc/V (p=0.004). The IgE titer at day 85 (cf. the right of FIG. 11), i.e. after the last aerosol challenge, reveals for NaHCO₃ application (cf. ▴ vs. Δ) and Lc/v application (▪ vs. □) significantly lower IgE levels for mice receiving s.c. boost compared to mice that did not receive s.c. boost. Only Lc/Dp2 fed mice ( vs. ◯) exhibit comparable levels. This result shows that feeding mice with Lc/Dp2 can attenuate IgE production with or without s.c. boosting.

FIG. 12 depicts a cytokine profile of spleen T-cells. Spleenocytes were cultured in the presence of Der p2 (10 μg/ml) for 3 days and subsequently for another 4 days in the presence of IL-2. On the ninth day of culture, T-cells were purified by Ficoll density centrifugation. Approximately 1×10⁵ cells/well were cultured for 48 h in the presence of 3×10⁵ APC/well and Der p2 (10 ug/ml). Control wells consisted of cells in the absence of Der p2. Culture media were obtained for cytokine profiling by ELISA. The Der p2-specific spleen T-cells from Lc/Dp2 fed mice compared to NaHCO₃ and Lc/V produced lower levels of Th-2 (IL-4, IL-5, IL-13) and pro-inflammatory cytokines (TNF-α, IFN-γ). However the Lc/Dp2 fed mice produced higher levels of T-regulatory cytokines (TGF-β and IL-10) compared to the Lc/V group (cf. also FIG. 24).

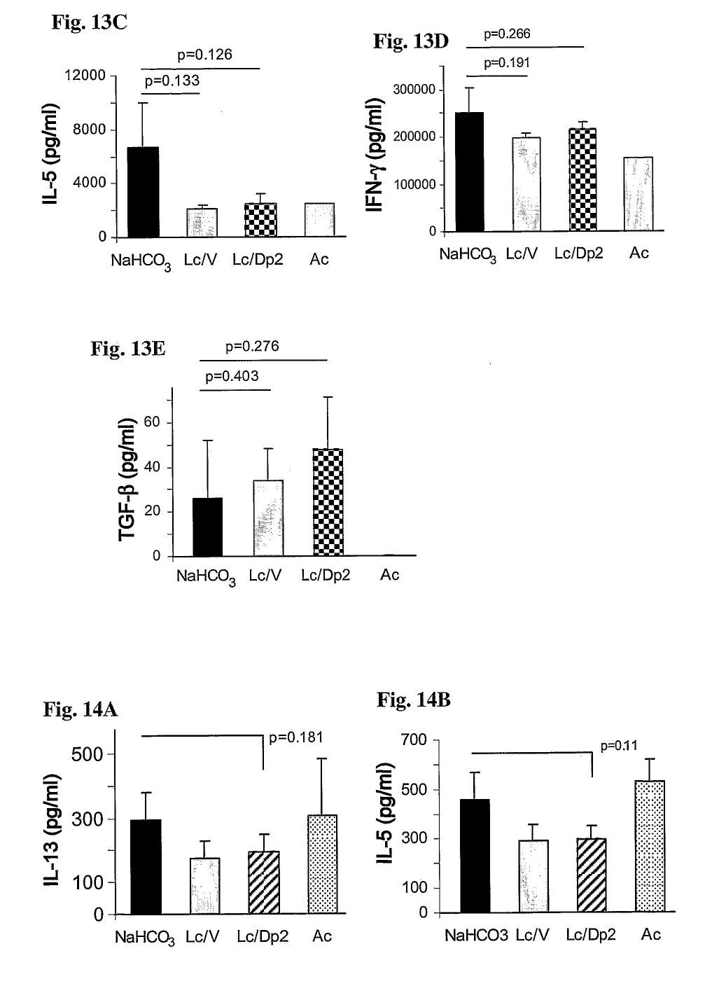

FIG. 13 depicts a cytokine profile of MLN cells. Total cells from MLNs of mice were culture for 48 h in 96-wells plate (3×10⁵ cells/well), in the presence of anti-CD3 and CD28. As shown, the L.c/V and Lc/Dp2 group produced significantly lower levels of IL-13 and a non-significant decrease in IL-4, IL-5, IFN-γ and IL-10 (levels similar to Ac mice) compared to the NaHCO₃ control group (A, B, C and D). In addition, MLN cells from Lc/Dp2 fed mice produced elevated level of TGF-β compared to control groups (D)

FIG. 14 shows the profiles of cytokines of the BALF. The BALF cytokine profile of mice fed with either Lc/V or Lc/Derp2 compared to the NaHCO₃ control indicated a decrease in effector Th2 cytokines (IL-13, IL-5), proinflammatory cytokine (TNF-α), TGF-β and the chemokine eotaxin.

FIG. 15 depicts BALF analysis and lung histology. The BALF and lung were obtained 24 h postaerosol challenge, for analysis and histological H&E staining. The Lc/Dp2 group compared to control groups of NaHCO₃ and Lc/V showed a reduction in BALF cell count, similar to mice receiving only aerosol challenge (Ac) (A). In addition, only Lc/Dp2 fed mice showed a reduction in recruitment of neutrophils (B). All groups exhibited similar percentages of macrophages, monocytes and eosinophils in the BALF. Lung tissues from two representative mice in each group were shown. H&E staining of lung sections from two aerosol control mice (G and H) showed a background of minimal airway inflammation in lung parenchyma with minimal inflammatory infiltrates in the bronchiolar spaces. However, lung tissues of mice from NaHCO₃ (A and B) and Lc/V (C and D) groups showed different degree (moderate to severe) of the airway infiltration inflammatory cells surrounding the airways and bronchiolar spaces. Comparatively, the Lc/Dp2 fed mice (E and F) showed a greater reduction in inflammatory infiltrates, having profile similar to that of Ac group.

FIG. 16 depicts the therapeutic regimen. In the therapeutic regimen, C57BL/6 mice were presensitized by epicutanous patching with Der p2 allergen at day 0, 14 and 28. At day 33, the level of Derp2-specific serum IgE and IgG1 for all mice was determined by ELISA and based on the IgE level, the mice were subsequently divided into three groups (n=6). At day 35, mice were fed orally with either NaHCO₃ buffer, Lc/V or Lc/Dp2 for 5 weeks, one dose per day for three consecutive days in a week. At day 55 and 62, mice received two subcutaneous immunizations of Der p2 (50 μg/mouse) and a week later mice were challenged twice by aerosol with Der p2 (1 mg in 10 ml PBS). Feedings are indicated by the filled triangles “▴” below the depicted time line. Approximately 24 h after the last challenge, mice were sacrificed and BALF obtained for differential cell count and cytokine analysis and the lung for H&E staining. In addition, cells from mesenteric lymph nodes (MLN) and spleen were obtained for primary and secondary T-cell culture and cytokine analysis.

FIG. 17 depicts Der p2-specific immunoglobulin responses. The kinetics of Der p2-specific sera IgE, IgG1 and IgG2a for all three groups of mice were as shown in FIGS. 17 (A, C and E). Mice in all three groups showed a decrease in Der p2-specific IgE one week after the start of feeding (A). The IgE level dropped significantly for Lc/V and Lc/Dp2 fed mice compared to control mice before and after two consecutive aerosol challenges (at day 69 and 77) (B). The Der p2-specific sera IgG1 was significantly elevated for these two groups compared to control mice at day 62 and 69 and remained unchanged even after airways challenged (C and D).

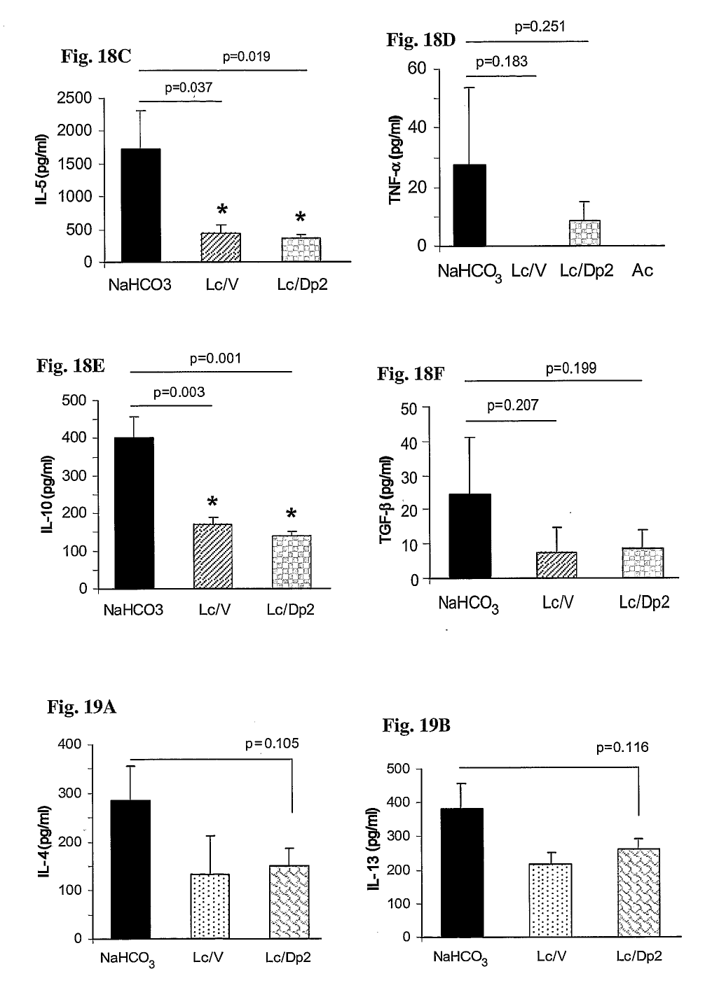

FIG. 18 shows a profile of selected cytokines of spleen T-cells. Splenocytes were culture in the presence of Der p 2 (10 μg/ml) for 3 days and subsequently for another 4 days in the presence of IL-2. On the ninth day of culture, T-cells were purify by Ficoll density centrifugation and approximately 1×10⁵ cells/well were cultured for 48 h in the presence of 3×10⁵ APC/well and Der p2 allergen (10 μg/ml). Control wells consisted of cells without Der p2. Culture media were obtained for cytokine profiling by ELISA. Both the Lc/Dp2 and Lc/V groups showed significant decrease in production of Th-2 cytokines (IL-5, IL-13, IL-10) and a non-significant decrease in IL-4 and TNF-α, compared to NaHCO₃ group (A-E). The TGF-β1 production was also decreased (F). No IFN-γ production is detected in all the three groups.

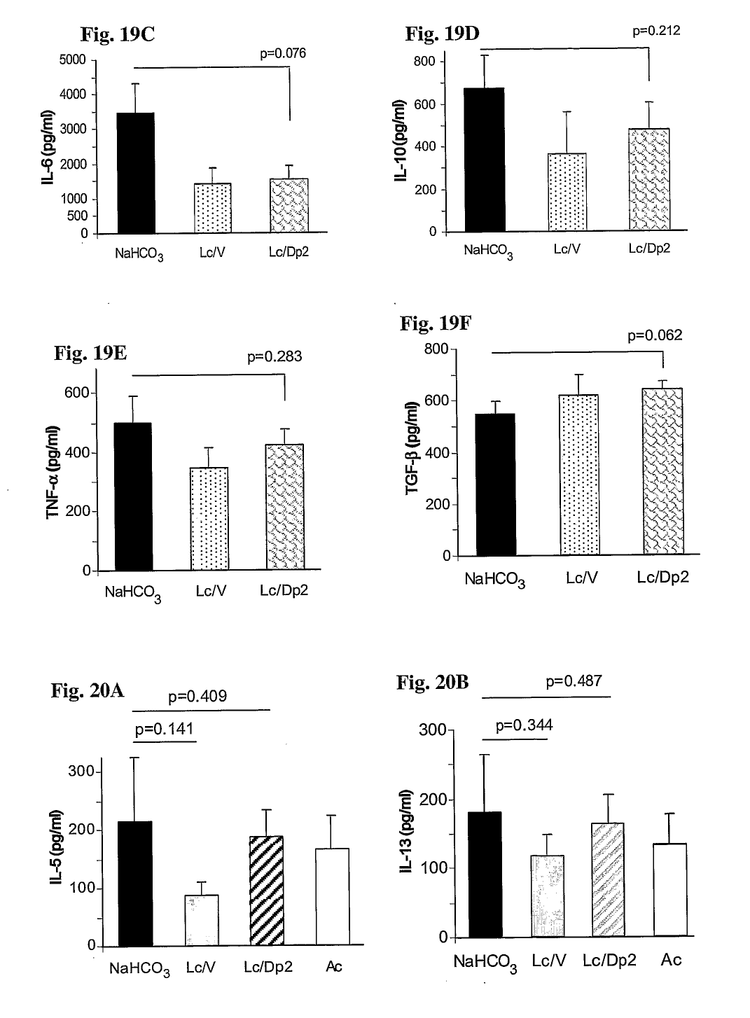

FIG. 19 shows a profile of selected cytokines of mesenteric lymph nodes cells. Total cells from mesenteric lymph nodes (MLNs) were culture for 48 h in 96-wells plate (3×10⁵ cells/well), in the presence of anti-CD3 and CD28. The MLNs cells from both the Lc/Dp2 and Lc/V groups compared to the NaHCO₃ group showed a cytokine profile showed a non-significant decrease in Th-2 and pro-inflammatory cytokines (IL-5, IL-4, IL-13, IL-10 and TNF-α). (A-E). In addition, the TGF-β1 production was elevated in cells from both the LcDp2 and Lc/V groups compared to control group.

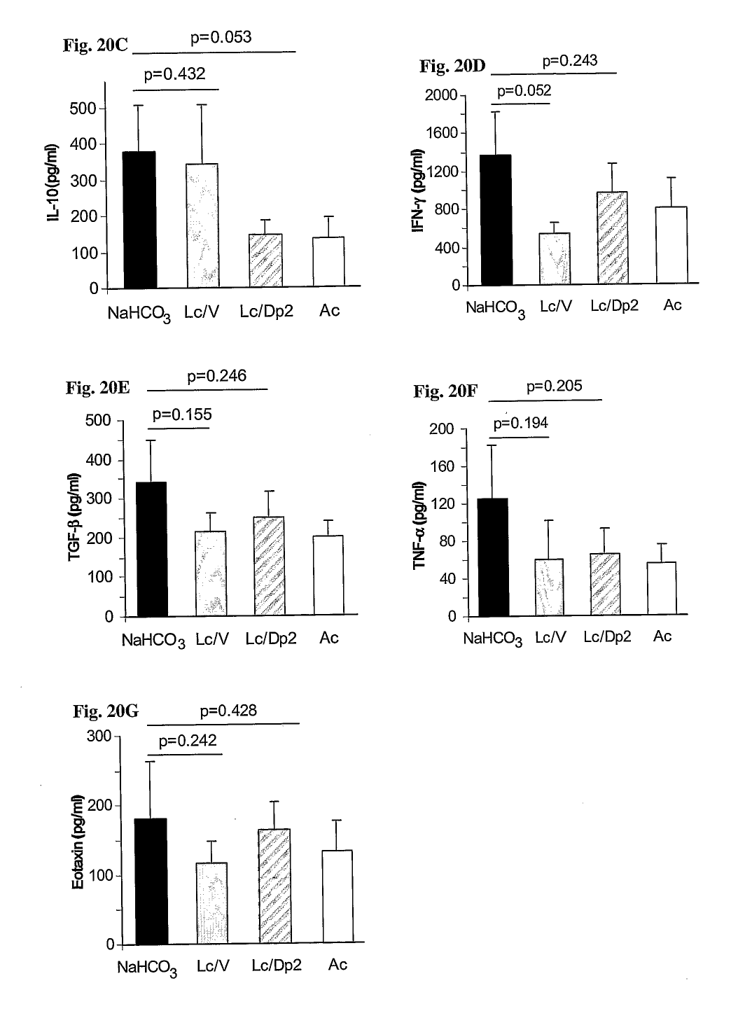

FIG. 20 shows the profiles of cytokines of the BALF. Mice were sacrificed 24 h after the last aerosol challenge and BALF were obtained for cytokine analyses. In both the Lc/V and Lc/Dp2 groups compared to NaHCO₃ group, there is non-significant decrease in both Th-2 and pro-inflammatory cytokines (IL-5, IL-13, IL-4, IFN-γ, TNF-α, TGF-β) and chemokine (eotaxin). Both groups have levels similar to that of Ac control group.

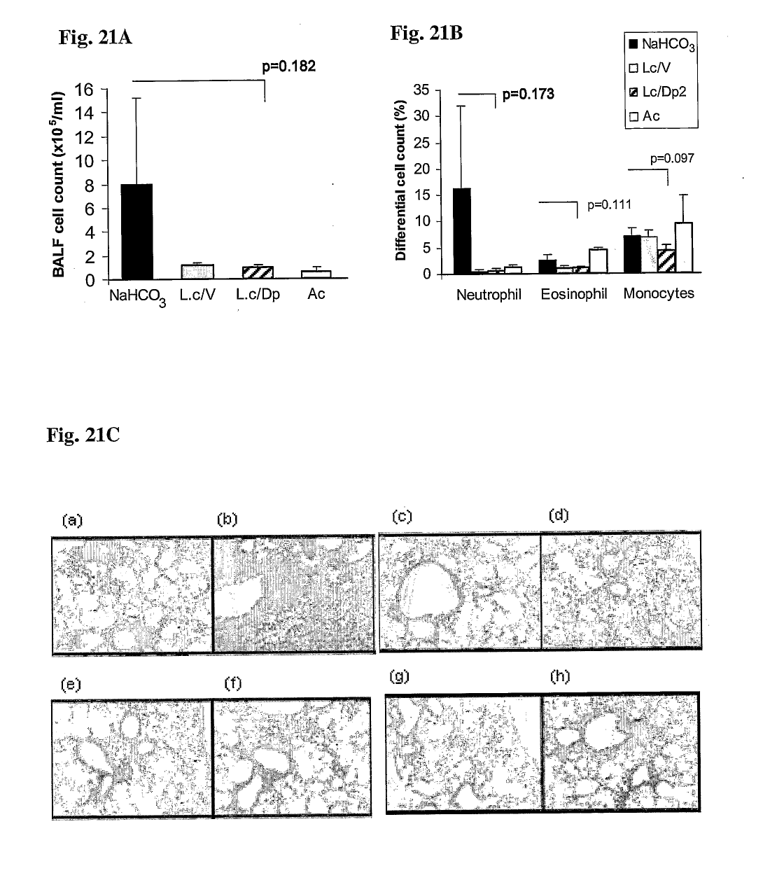

FIG. 21 depicts the pathophysiological changes in the lungs and a bronchoalveolar fluid analysis. Analyses of the BALF indicated that both Lc/V and Lc/Dp2 have similar number of total infiltrating cells with the aerosol control group (Ac) and is non-significantly lower than observed in NaHCO₃ group (A). Differential cell analyses indicated a reduction in neutrophils and eosinophils for both of these groups, no changes in lymphocytes and macrophages counts (B). Only the Lc/Dp2 group exhibit slight reduction in monocytes count in the BALF compared to the other groups. Lung tissues from two representatives in each group were shown. H&E staining of lung sections from two aerosol control mice (G and H) showed a background of minimal airway inflammation in lung parenchyma with minimal inflammatory infiltrates in the bronchiolar spaces. However lung tissues of mice from NaHCO₃ group exhibited different degree (moderate to severe) of airway inflammatory infiltrating cells surrounding the airways and bronchiolar spaces (A and B). Comparatively, both the Lc/V (C and D) and Lc/Dp2 (E and F) fed mice showed a greater reduction in inflammatory infiltrates, having profile similar to that of Ac mice.

FIG. 22 depicts a therapeutic regimen using live recombinant lactobacilli. In the therapeutic regimen, two groups of C57BL6 mice were presensitized by epicutanous patching with recombinant yeast-derived Der p 2 allergen at day 1, 14 and 28. After resting for 14 days, only IgE responders were divided equally into two groups (Lb vs buffer, n=6) for treatment studies. Live recombinant Der p 2 (Lc/Dr p2) were fed to the Lb group. Mice were fed daily for a total of 4 weeks between day 42 and 70 ( [symbol] ). At day 80 both groups were challenged by aerosol with Der p 2 (1 mg in 10 ml PBS). Readings of the enhanced pause (pEnh), an indicator of bronchoconstriction, were taken approximately 24 h. Mice were sacrificed at d 82 for primary and secondary T-cell culture.

FIG. 23 illustrates the systemic immunoglobulin response. After 7 days of active feeding, a 41% attenuation of the Der p2 specific serum IgE (A) was observed in L. casei Shirota/Dp2 group compared to just 27% in the NaHCO₃ control group. In addition, the Der p2-specific serum-IgG1 (B) was significantly attenuated in the L. casei/Dp2 group after 14 days of active feeding, while the NaHCO₃ control group only showed attenuation of IgG1 after 21 days of feeding. In the profile of splenic cytokines from secondary culture in (C) white bars represent the mean of data from 5 control mice. Black bars represent the mean of data obtained from pooled cells of 6 fed mice. The cytokines profile of the spleen T-cells from both buffer-fed mice and pooled cells of recombinant lactobacilli-fed mice (the pooled cells showed poor proliferation, a marker for T regulatory cells) showed no difference in the TH1 (IFNγ) and TH2 (IL-5 and IL-13) cytokines formation. However, mice fed with L. casei Shirota/Dp2 showed an increase in T-regulatory cytokines (IL-10 and TGF-β) production. Buffer-fed mice (n=6) vs pooled cells of recombinant lactobacilli-fed mice.

FIG. 24 depicts a treatment model hypothesis, which is understood not to be meant in any way binding as to a, or the, effect underlying the methods of the present invention. Epicutaneous patching may result in reduced levels of IL-5 from TH2 cells. However, the recombinant lactobacillus may cause an increase in levels of cytokines of Tr cells.

FIG. 25 shows a schematic of the experimental protocol for the analysis of the effect of subcutaneous priming of on mice. Mice were primed by subcutaneous injection with a low dose (LD, 10 μg) or a high dose (HD, 50 μg) of Der p 2 on day 0, 4, 8, followed by a boost with Der p 2 on day 28. Mice were subjected to aerosol inhalation with 0.1 mg/ml of Der p 2 in PBS for 30 min on day 56, 58, 60 and 62, and sacrificed on day 64 for T-cell cytokine analysis. Control mice were subjected to aerosol inhalation alone.

FIG. 26 depicts the kinetics of Der p 2-specific humoral response in mice. Mice were primed by subcutaneous injection with a low dose (white squares, 10 μg) or a high dose (black squares, 50 μg) of Der p 2, followed by a boost with a low dose of Der p 2 and aerosol inhalation. The Der p 2 specific IgE (A), IgG1 (B), and IgG2a (C) titers of mice primed with low dose or high dose of Der p 2 were determined by ELISA. Data are expressed as mean±SEM (n=8). *: p<0.05, two-tailed Student's t-test for independent samples.

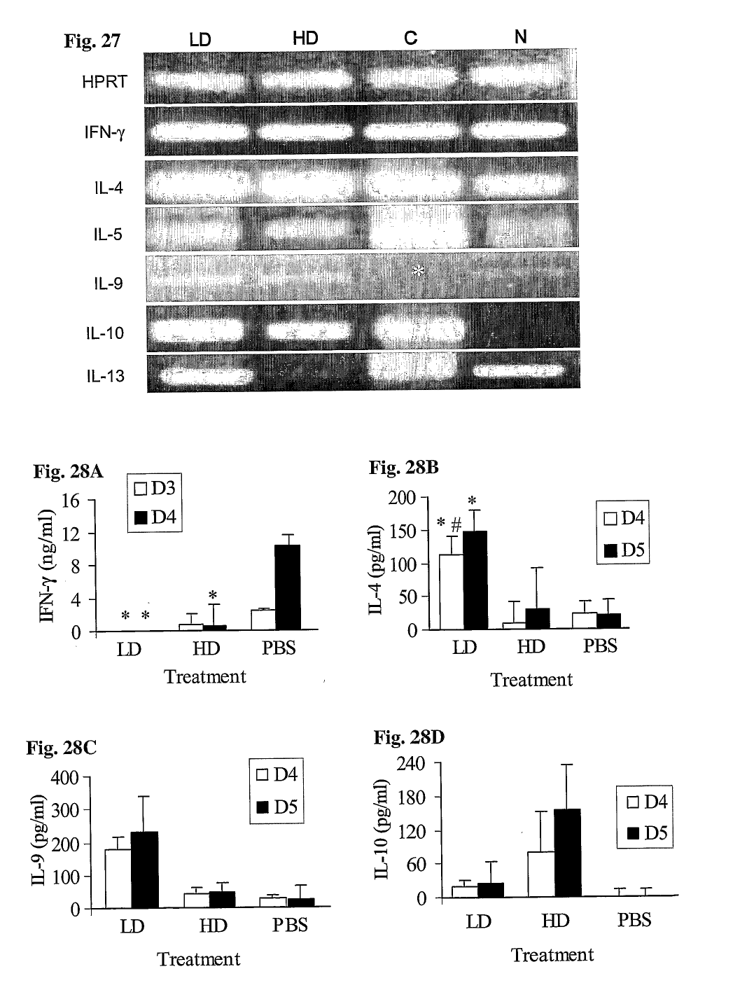

FIG. 27 depicts an RT-PCR analysis on cytokine profiles of splenic CD4⁺ T-cells (cf. also Tab. 1). Mice primed with low dose (LD, cf. top of the figure) and high dose (HD) of Der p 2, and control mice (C) were subjected to aerosolized Der p 2 exposures and sacrificed on day 64. Splenocytes were cultured with Der p 2 for 10 days. Purified CD4⁺ T-cells of Der p 2 cultured splenocytes were stimulated with anti-CD3 and anti-CD28 for 24 hr, and total RNA were isolated for the analysis of cytokine expression. Purified CD4⁺ T cells from age-matched naive mice (N) were included for comparison. Each band shows the amplified cytokine kit from pooled spleens of eight mice. (*: not carried out)

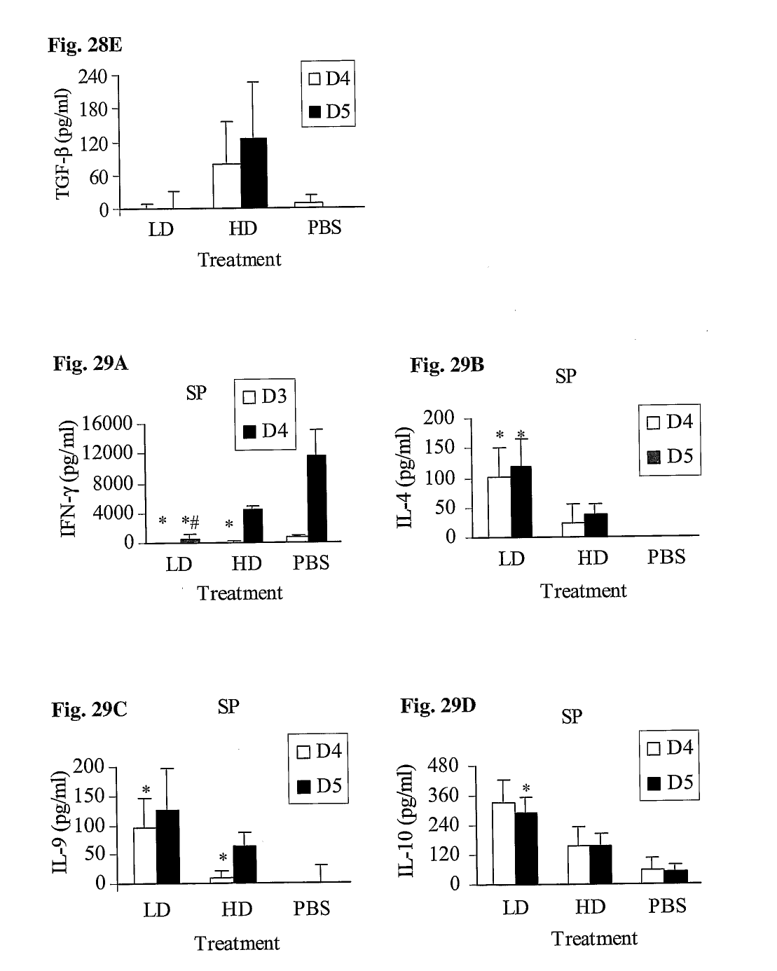

FIG. 28 depicts cytokine profiles of lymph nodes in culture. Mice were primed with low dose (LD, 10 μg) or high dose (HD, 50 μg) of rDer p2 protein, or PBS on day 0, 4, 8 and sacrifice on day 10. Lymph nodes were harvested and cultured for 3 to 5 days in the presence of rDer p 2 protein. Culture supernatants were collected and analyzed for IFN-γ (A), IL-4 (B), IL-9 (C), IL-10 (D), and TGF-β (E) formation by ELISA. Results shown are representative of 2 independent experiments. Mean±s.e.m. (n=4). * comparison with PBS, # comparison with HD, p<0.05, two-tailed Student's t-test for independent samples.

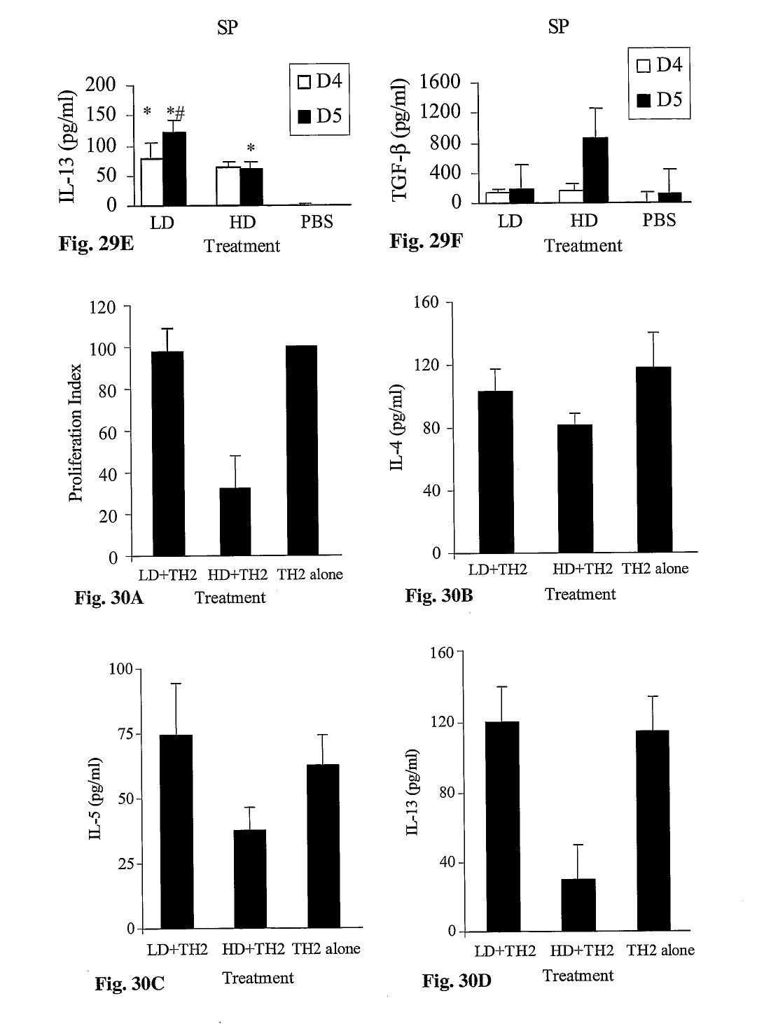

FIG. 29 shows cytokine profiles of SP cultures. Mice were primed by subcutaneous injections with low dose (LD, 10 μg) or high dose (HD, 50 μg) of Der p 2, or with PBS on day 0, 4 and 8. SPs were harvested on day 10 and cultured with 10 μg/ml of rDer p2 protein. Supernatants were collected on day 3-5 and analyzed for IFN-γ (A), IL-4 (B), IL-9 (C), IL-10 (D), IL-13 (E) and TGF-β (F) formation by ELISA. Results shown are representative of two independent experiments, mean±s.e.m. (n=4); * comparison with PBS, p<0.05; # comparison with HD, p<0.05, two-tailed Student's t-test.

FIG. 30 depicts the proliferation and cytokine response of antigen-specific TH2 cells upon co-culture with CD4⁺CD25⁺ cells. Antigen-specific TH2 cells were derived from splenocyte cultures of mice, patched with 50 μg of rDer p2 protein. TH2 cells were cultured with splenic CD4⁺CD25⁺ T cells of mice primed with LD (LD+TH2) or HD (HD+TH2) of rDer p2 protein, or alone (TH2 alone) in the presence of rDer p 2 protein for 5 days and assayed for the proliferation response. Proliferation ratio is expressed as the index of TH2 cells alone (a). Supernatant was harvested at t=72 hrs and assayed for IL-4, IL-5 and IL-13 production (b,c,d). Results showed the average of 3 independent experiments, mean±s.e.m.. * comparison with HD; # comparison with TH2 alone. Student t test, p<0.05.

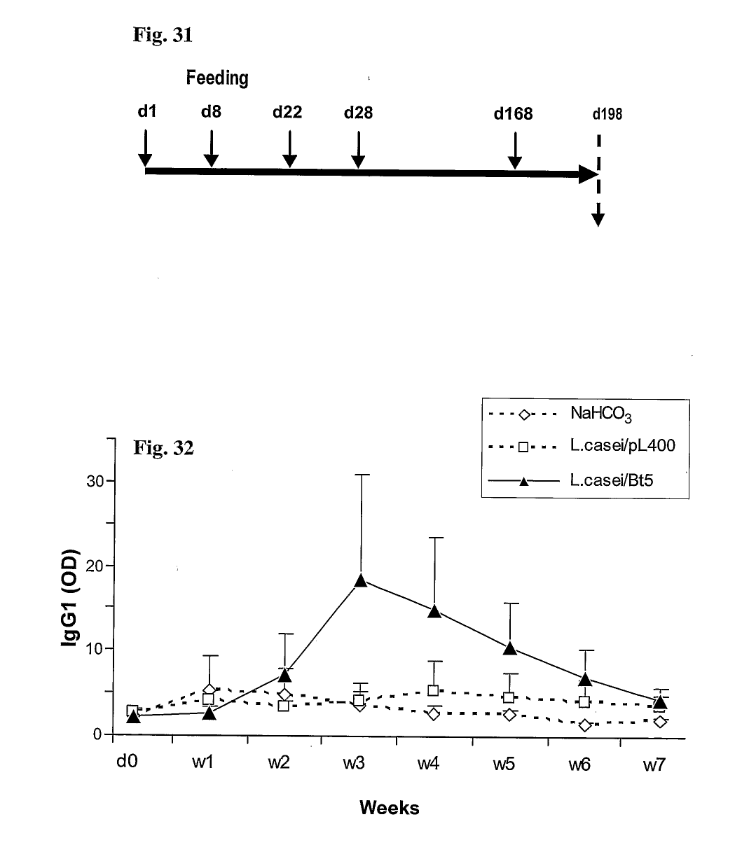

FIG. 31 depicts a regimen used in animal studies with recombinant L. casei Shirota expressing the Blo t 5 allergen. Three groups of Balbc/J mice were examined: NaHCO₃ (n=4), L. casei/pL400 (n=4) and L. casei Shirota/Blo t 5 (L. casei/Bt 5; n=4). Mice were fed with 1×10⁹ cfu/mouse each day for four consecutive days per week (indicated by arrows). Total feeding each mouse received were 20 doses of 1×10⁹ cfu. Mice were bled weekly up to 7 weeks and Blo t 5-specific serum immunoglobulins were assayed by ELISA. Mice were sacrificed on day 198 and T-cells from Peyer's patches and spleen were obtained for cytokine analysis.

FIG. 32 depicts the data of Blo t 5-specific serum immunoglobulins obtained by ELISA. There were significant levels of Blo t 5 specific IgG1 detected, whereas no significant levels of IgE and IgG2a were detected.

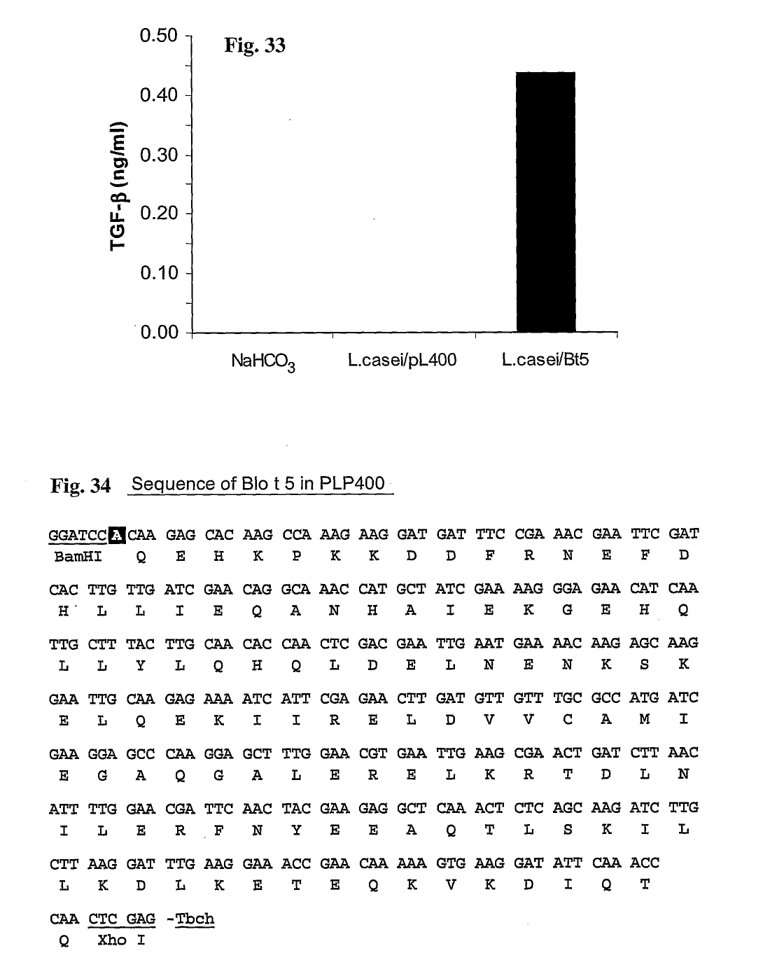

FIG. 33 depicts the cytokine analysis of sacrificed mice (cf. FIG. 34). Significant levels of regulatory cytokine TGF-β were detected in T cells from Peyer's patches of the L. casei Shirota/Blo t 5 fed mice only, indicating that the live recombinant L casei Shirota/Blo t 5 actively induced production of T regulatory cytokines by T cells in Peyer's patches.

FIG. 34 depicts the nucleic acid sequence (upper line) and the amino acid sequence (lower line, one letter code) of the allergen Blo t 5, used in examples illustrating the present invention, in the expression vector pLP400. The additional base “A” at the 5′ end (inverted) was introduced to shift the gene inframe with the vector's indigenous start codon.

FIG. 35 depicts the nucleic acid sequence (upper line) and the amino acid sequence (lower line, one letter code) of the allergen Der p 2, used in examples illustrating the present invention, in the expression vector pLP500. The additional base “A” at the 5′ end (inverted) was introduced to shift the gene inframe with the vector's indigenous start codon. The sequence included the anchor sequence of L. casei (“anchor”).

Table I depicts TH2 cytokine profiles of splenic CD4⁺ T-cells by real-time PCR. Mice primed with low dose and high dose of Der p 2, and control mice were subjected to aerosolized Der p 2 exposures and sacrificed on day 64. Splenocytes were cultured with Der p 2 for 10 days. Purified CD4⁺ T-cells of Der p 2 cultured splenocytes were stimulated with anti-CD3 and anti-CD28 for 24 hr, and total RNA were isolated for the analysis of cytokine expression. Purified CD4⁺ T cells from age-matched naive mice (N) were included for comparison. Representative data of duplicate experiments are shown. *:Cytokine ratio is determined by dividing the value of cytokine/HPRT of the experimental group (sample) with the naive group (calibrator).

### EXAMPLES

Mice used in the following illustrative examples were C57BL/6 mice, 3-4 weeks of age, purchased and housed in the Animal Holding Unit in National University of Singapore. The IACUC on animal welfare approved all animal protocols used in this study. Stocks of wildtype and recombinant L. casei Shirota strain or L. rhamnosus GG used in the following examples were kept in aliquots containing 50% glycerol and stored at −70° C.

Statistical comparisons were performed by analysis of means using Student's t-test. All values are shown as mean±standard deviation (SD). A value of p<0.05 was regarded as significant.

### Example 1

### Construction of Recombinant L. Casei Expressing Enhanced Green Fluorescence Protein (eGFP)

The eGFP gene was amplified by polymerase chain reaction (PCR) using Expand High fidelity DNA polymerease (Boehringer) and synthetic primers BamHI-eGFP/f [5′-CCC CCG GATi CCA gtg agc aag ggc gag gag ctg-3′, SEQ ID NO: 3] and eGFP-xhoSac/r [5′-CCC CCC ctc gag CTT GTA CAG CTC GTC CAT GCC GAG-3′, SEQ ID NO: 4]. The resultant PCR kits containing a Bam HI site at the 5′ end and a Xho I/Sac I site at the 3′ end was subsequently subcloned into the Bam HI and Sac I site of pTUAT (cf. FIG. 1). The Bam HI/Nhe I fragment containing eGFP-uidA-Thch was exchanged with the Bam HI/Nhe I fragment expression-secretion vectors of the pLP500 (cf. FIG. 1).

The uidA gene was then removed by digestion with Xho I, resulting in the expression construct pLP500-eGFP.

### Example 2

### Cloning of Der P2 Gene and Blo t 5 Gene into Lactobacillus/E. Coli Shuttle Vector

A 441 bp fragment of Der p2 cDNA was amplified by PCR using Expand High fidelity DNA polymerase (Boehringer) and synthetic primers, Dp2Bam/f [5′-CCCCCGGATCCAGATCAAGTCGATGTCAAAGATTGTGC-3′, SEQ ID NO: 5] and Dp2xhoSac/r [5′-CCCCCCGAGCTCCTCGAGATCGCGGATTTTAGCATGAGTAGC-3′, SEQ ID NO: 6]. The resultant PCR kit of the Der p 2 gene containing a Bam HI and a Xho I/Sac I site, at the 5′- and 3′-end respectively, had the sequence: 5′-GGATCC A GAT CAA GTC GAT GTC AAA GAT TGT GCC AAT CAT GAA ATC AAA AAA GTT TTG GTA CCA GGA TGC CAT GGT TCA GAA CCA TGT ATC ATT CAT CGT GGT AAA CCA TTC CAA TTG GAA GCC GTT TTC GAA GCC AAC CAA AAC ACA AAA ACC GCT AAA ATT GAA ATC AAA GCC TCA ATC GAT GGT TTA GAA GTT GAT GTT CCC CGT ATC GAT CCA AAT GCA TGC CAT TAC ATG AAA TGC CCA TTG GTT AAA GGA CAA CAA TAT GAT ATT AAA TAT ACA TGG AAT GTT CCG AAA ATT GCA CCA AAA TCT GAA AAT GTT GTC GTC ACT GTT AAA GTT ATG GGT GAT GAT GGT GTT TTG GCC TGT GCT ATT GCT ACT CAT GCT AAA ATC CGC GAT CTC GAG (SEQ ID NO: 1). The PCR kit was subcloned into the Bam HI and Sac I site of an intermediate pTUAT vector (FIG. 1B). Subsequently the Bam HI/Nhe I fragment containing Der p2-uidA-anchor-Tbch was exchanged with the Bam HI/Nhe I fragment of a Lactobacillus/E. coli shuttle vector pLP500 (FIG. 1A). The uidA gene was then removed by digestion with Xho I and re-ligated, resulting in generation of pLP500/Dp2-anchor expression construct which was subsequently verified by nucleotide sequencing.

A corresponding approach using the same genetic engineering technology by means of suitable materials known in the art was carried out for Blo t 5, using Lactobacillus/E. coli shuttle vector pLP400. The respective sequence of the Blo t 5 gene in the vector pLP400 encoding a Blo t 5 protein that lacks the 17 N-terminal amino acids was: 5′-GGATCC A CAA GAG CAC AAG CCA AAG AAG GAT GAT TTC CGA AAC GAA TTC GAT CAC TTG TTG ATC GAA CAG GCA AAC CAT GCT ATC GAA AAG GGA GAA CAT CAA TTG CTT TAC TTG CAA CAC CAA CTC GAC GAA TTG AAT GAA AAC AAG AGC AAG GAA TTG CAA GAG AAA ATC ATT CGA GAA CTT GAT GTT GTT TGC GCC ATG ATC GAA GGA GCC CAA GGA GCT TTG GAA CGT GAA TTG AAG CGA ACT GAT CTT AAC ATT TTG GAA CGA TTC AAC TAC GAA GAG GCT CAA ACT CTC AGC AAG ATC TTG CTT AAG GAT TTG AAG GAA ACC GAA CAA AAA GTG AAG GAT ATT CAA ACC CAA CTC GAG (SEQ ID NO: 2). The PCR fragment was subcloned into the Bam HI and Sac I site of an intermediate pTUAT vector (FIG. 1B). Subsequently the Bam HI/Nhe I fragment containing Blo t 5-uidA-Tbch was exchanged with the Bam HI/Nhe I fragment of a Lactobacillus/E. coli shuttle vector pLP400 (FIG. 2). The uidA gene was then removed by digestion with Xho I and re-ligated, resulting in generation of pLP400/Bt 5-anchor expression construct which was subsequently verified by nucleotide sequencing.

All plasmid DNA and constructs were maintained and propagated in E. coli host cells.

### Example 3

### Confocal Analysis of L. Casei Shirota-eGFP

L. casei Shirota cells containing the pL500-eGFP construct (L. casei Shirota-eGFP) and pL500 vector were respectively cultured in MRS medium (Difco Laboratories Detroit) containing 5 μg/ml erythromycin, at 37° C. in a 5.0% CO₂ incubator. When the culture reached OD₆₉₀ₙₘ of 0.6 and 1.8, a total of 0.5 ml was harvested and washed twice in PBS (pH 7.4). Cells were resuspended in 1 ml of PBS and analyzed under confocal microscopy. L. casei Shirota containing the pL500 vector served as a negative control.

### Example 4

### Translocation of L. casei Shirota-eGFP in Mice Peyer's Patches

To examine the in-vivo translocation L. casei Shirota-eGFP to Peyer's patches in mice, 6-weeks-old Balbc/J mice were orally administered with 1×10⁹ colony forming units (cfu) of L. casei Shirota-GFP or L. casei Shirota-pL500 per mouse for four consecutive days. On the fifth day, the mice were sacrificed and Peyer's patches tissue of individual mice were obtained for cryostat tissue section for immunohistochemistry and transmission electron microscopy.

Peyer's patches embedded in OCT and quick-frozen in liquid Nitrogen. Cryostat tissue sections of 2 um in thickness were placed on silanized slides and fixed in 100% acetone (+0.02% H₂O₂) for 10 min at 4° C. The sections were air dried and subsequently washed three times in PBST (0.05% Tween 20). Fixed sections were then blocked in Ultra V Block (Lab Vision Corp, Fremont Calif., USA) for 5 min at room temperature. The blocking solution was removed and respective or combination of antibodies [APC conjugated anti-mouse THY-1.2, phycoerythrin (PE)-conjugated anti-mouse CD19 (BD Biosciences)] diluted 1:100 in PBST containing 1% BSA was added and incubated overnight at 4° C. in a moist chamber. The following day, the sections were washed three times in PBST (5-10 min each). The slides were mounted in Fluor Save™ Reagent (Calbiochem) and observed under confocal microscopy.

### Example 5

### Transmission Electron Microscopy (TEM)

TEM of Peyer's patches tissues from orally administered mice were carried out to show translocation of intact L. casei Shirota and not the eGFP protein. Briefly, tissue sections of Peyer's patches from mice fed with L. casei Shirota-eGFP (4 times over a period of 4 days) were fixed in 2.5% of glutaraldehyde overnight at 4° C. These samples were post-fixed with 1% osmium tetroxide in cacodylate buffer at room temperature, stepwise dehydrated in increasing concentrations of ethanol, followed by a final dehydration in 100% propylene oxide. Samples were incubated in a 1:1 mixture of propylene oxide:epoxy resin and finally embedded in epoxy resin. Ultra-thin sections were mounted on copper grids, stained with uranyl acetate and lead citrate and observed on a transmission electron microscope.

### Example 6

### Electroporation and Heterologous Expression of Der p2 in L. Casei Shirota

Initially, L. casei Shirota was cultured in MRS medium (Difco Laboratories Detroit) at 37° C. in a 5.0% CO₂ incubator. Competent cells of L. casei were prepared from cells at mid-log phase (OD₆₉₀ₙₘ=0.6), washed in pre-chilled wash buffer (50 mM KH₂PO₄—K₂HPO₄ (pH 7.4); 0.3 M Sucrose, 1 mM MgCl₂) and resuspended in chilled electroporation buffer (952 mM Sucrose, 3.5 mM MgCl₂). Plasmid DNA pLP500 or pLP500/Dp2-anchor (1 μg) was added to 100 μl cells suspension and transferred to a 0.2 cm cuvette for electroporation using the Gene Pulser II, BioRad (conditions: 2.5 kV potential; 25 μF capacity, 200 ohm resistance). After pulsing, 900 μl MRS medium was added and cells incubated for 3 h before plated on MRS agar containing erythromycin (5 μg/ml).

Extraction of plasmid DNA from transformed L. casei Shirota and heterologous expression in L. casei Shirota under constitutive promoter of L-(+)-lactate dehydrogenase gene was performed as previously described by Maassen et al. (Vaccine. (1999) 17, 17, 2117-2128). A total of 5 mls of transformed bacteria culture was harvested and pellet washed once in PBS (pH 7.4). The bacteria pellet was digested in PBS buffer containing lysozyme (Sigma) for 1 hr on ice and thereafter DNA plasmid extraction was carried out using the Wizard DNA Miniprep kit (Promega).

For heterologous expression study, an overnight culture of Lc/Dp2 or Lc/V was used to innoculate MRS broth (1:700 dilutions) containing 5 μg/ml erythromycin and 1% glucose (w/v) in a 50 ml Falcon tube. Cultures were grown overnight and cells were harvested at OD₆₉₀ₙₘ of >1.0 and washed once in PBS. Cells were resuspended in lysis buffer (PBS containing 1% Tween 20 and 1 mM PMSF), sonicated (10 amplitude microns, 30 s on/30 s off, 2 mins) and the soluble fraction were separated on a SDS-PAGE for Western immuno-blot assay using Der p2 monoclonal antibody for detection.

### Example 7

### Co-Culture of Mouse T-Cells from Spleen and Mesenteric Lymph Nodes with Wildtype L. casei Shirota

Cytokine profiles were determined for mouse T-cells after co-cultured with wildtype L. casei Shirota. Approximately 1×10³ (mesenteric or sorted CD3⁺ cells from spleen) or 3×10³ (total spleen) were added per well for co-culture. A fresh culture of L. casei Shirota was grown in MRS broth (Difco) at 37° C. in 0.5% CO₂ incubator until OD₆₉₀ₙₘ reached 0.6. An aliquot of the culture was washed twice in PBS, once in RPMI 1640 and finally resuspended in T-cell culture media. A culture aliquot was washed twice in PBS, once in RPMI 1640 and finally resuspended in T-cell culture media. Co-culture were carried out in two sets of duplicate wells (in a final volume of 200 μl/well) on a 96-well culture plate and at ratio of T-cells:L. casei Shirota of 1:0; 1:0.5; 1:1; 1:2; 1:5. Culture supernatants were obtained at 16 h and 24 h post co-culture and assayed for TGF-β cytokine level using ELISA.

### Example 8

### Preparation of Lc/V and Lc/Dp2 for Feeding

A stock of heat-killed Lc/V and Lc/Dp2 required for the entire feeding experiment was prepared. A fresh culture of Lc/V or Lc/Dp2 was grown in MRS broth (Difco) at 37° C. in 0.5% CO₂ incubator and quantitated by spectrophotometer, based on the optical density (OD) 1.0 at 690 nm equivalent to 5×10⁸ cfu/ml of culture. The required amount of Lc/V or Lc/Dp2 cultures was centrifuged at 3,500 rpm for 10-15 min and cell pellet washed twice in PBS (pH7.0) followed by a final wash in 0.2 M NaHCO₃ (pH 8.4). The cells were subsequently resuspended in 0.2 M NaHCO₃ (pH 8.4) buffer to a final concentration of 109 cfu/100 ul and aliquoted in 400 μl per micro reaction tube (Eppendorf). The bacteria were then heat-killed at 95° C. for 30 min in a Thermomixer (Eppendorf) and subsequently stored frozen at −70° C. until further use. The viability of cells was tested by culturing on an MRS plate.

### Example 9

### Detection of Der p2-Specific Immunoglobulin Responses

The levels of Der p 2-specific IgE and IgG1 were determined by ELISA. Briefly, mouse sera were incubated in duplicate with Der p 2 (2 μg/ml) coated wells for overnight at 4° C. Biotin-conjugated monoclonal rat anti-mouse IgE (R19-15) and anti-mouse IgG1 (G1-1.5) were used for detection and followed by addition of ExtrAvidin-alkaline phosphatase. Signals were developed by addition of p-Nitrophenylphosphate (PNPP) substrate. ELISA index unit was defined as the OD₄₀₅ₙₘ reading corresponding to the reading of 1 ng/ml of purified mouse IgE or IgG1 in a sandwich ELISA with anti-mouse Igκ as the capture antibody in the same plate. All the antibodies used were purchased from Pharmingen (Pharmingen, San Diego, Calif.).

### Example 10

### T-Cell Cytokine Profiling and Proliferation Assay

T-cell cultures were carried out in RPMI 1640 medium supplemented with 10% heat-inactivated bovine calf serum (StemCell Technologies Inc.), 2 mM L-glutamine, 1 mM sodium pyruvate, 100 U/ml penicillin and 100 μg/ml streptomycin (Hyclone Laboratories, Logan, Utah) and 5.5×10⁻² mM 2-mercaptoethanol (Life technology, Grand Island, N.Y.).

Briefly, single-cell suspensions were prepared from spleen and red blood cell lysis was performed. Spleenocytes (30×10⁶ cells/well) were cultured in 6-wells plate containing 10 ml of supplemented RPMI 1640, in the presence of 10 μg/ml of Der p2 for three days. On the third day, 5 ml of culture media from each well were replaced with fresh culture media containing IL-2 (10 U/ml) and cells were maintained as such, with replacement of media at every 2 days for a total nine days of culture. At day nine, Der p2-specific T-cells were purified by Ficoll and cells reactivated in duplicate on 96-well round-bottomed plates (Costar, Corning, N.Y.), each well contain 1×10⁵ purified T-cells, 3×105 mytomycin treated APC and 10 μg/ml Der p2 in a final volume of 200 μl/well. After 48 h of culture, supernatant were collected and stored at −20° C. Mesenteric lymph nodes (MLNs) cells were cultured in wells coated with 5 μg/ml anti-CD3 (clone: 145 2C11) and anti-CD28 (clone: 37 [symbol] 51) (both clones from BD Pharmingen, Oxford, UK). After 72 h, supernatants were collected and stored at −20° C. Cytokine profiling was performed in duplicate by ELISA using antibodies pairs (Pharmingen) to assay for the presence of IL-4, IL-5, IL-13, TNF-α, TGF-β and IL-10 cytokines. Purified antibodies to mouse IL-4, IL-5, IL-10, IL-13, TNF-α, and TGF-β antibodies were coated on 96-well plate at 2 μg/ml. Biotinylated polyclonal antibodies to the respective mouse IL-4, IL-5, IL-10, IL-13, TNF-α, and TGF-β antibodies were used as recommended by the supplier. Recombinant mouse cytokines were used as standards in the ELISA assay. All antibodies and recombinant cytokines were from BD Biosciences PharMingen (San Diego, Calif.) unless stated otherwise.

The T-cell proliferation assay was performed using Peyer's patch cells (1×10⁵ cells/well) co-culture with APCs (3×10⁵ cells/well) with or without 5 μg/ml or 10 μg/ml rDer p2, for 72 hrs. Approximately 18 hr before harvest, cells were pulsed with 1 μCi of [3H]-thymidine (NEN Life Science, Boston, Mass.). Cells were harvested to a glass fiber filter (Skatron instruments AS, Lier, Norway). After adding the scintillation fluid (Amersham Biosciences Corp), proliferation was measured by the liquid scintillation counter (Beckman Coulter, Inc. Fullerton, Calif.). The proliferation index is expressed as ratio of thymidine incorporation by cells in the presence to that in the absence of rDer p 2.

### Example 11

### BALF Analysis and Lung Histology

Mice were injected i.p. with a lethal dose of a mixture containing 1.25 mg/ml midazolam, 2.5 mg/ml fluanisone and 0.079 mg/ml fentanyl citrate. The trachea were exposed and cannulated by tracheostomy (20G cannula) and the lung was lavaged with 0.8 ml of ice-cold Hank's balanced salt solution (HBSS without calcium and magnesium) for three times and the fluid volume pooled. Total cell count and duplicate cytospin for differential cell count of BALF were performed. Briefly the volume of BAL samples were determined, the samples were centrifuged (700 g for 5 min at RT), resuspended at 1×10⁵ cells in 100 μl (PBS/10% FCS) and cytospins were made by cytocentrifugation (300 g for 3 min) onto poly (1-lysine)-coated slides (Cytospin 5 Thermo Shandon Inc., Pittsburgh, Pa.). Slides were air-dried, fixed in methanol and stained using standard procedures (Merck, UK). Differential cell counts were performed in duplicate on coded slides for 500 cells from each sample. Levels of IL-5, IL-13, TNF-α, TGF-β and eotaxin were determined by ELISA.

Lungs were removed, washed in PBS and fixed in 10% formalin. Lungs were embedded in paraffin and sections (2 um thick) were assessed for general morphology and cellular infiltration using haematoxylin and eosin (H&E).

### Example 12

### Prophylactic Regimen in an Asthma Mouse Model

The present example illustrates the use of orally administered heat-killed recombinant L. casei Shirota expressing Der p 2 (Lc/Dp2) in combination with s.c. boosting of Der p 2 in a prophylactic regimen (cf. FIG. 10). The present example is based on a prime-boost strategy using a Der p2 induced experimental asthma mouse model, generated via sensitization by skin patching and aerosol challenged with Der p2. The present example also illustrates, how the efficacy of orally administered heat-killed recombinant L. casei Shirota expressing Der p2 in combination with s.c. boosting of Der p 2 in a prime-boost strategy can be evaluated using a prophylactic regimen on the Der p 2-induced asthma model (cf. FIG. 10). The control groups consisted of mice fed with either NaHCO₃ buffer or heat-killed Lc/V. In the present example heat-killed instead of live recombinant lactobacilli were used due to the ease of lactobacilli preparation and to satisfy safety requirements on the use of live genetically modified organisms (GMOs). It should nevertheless be noted (cf. also above) that the lactobacillus may be of any desired activity.

Oral feeding was carried out using autoclaved gavage needles (Popper & sons, inc., NY). An aliquote of frozen heat-killed Lc/V or Lc/Dp2 described in Example 8 were thawn to room temperature and each mouse received one dose of 100 μl containing 109 heat-killed Lc/V or Lc/Dp2 or 100 μl of NaHCO₃ buffer per day, for three consecutive days in a week, for the entire duration of the study. At day 11 and 18, all mice were immunized subcutaneously with Der p2 (50 μg/mouse). All mice were then sensitized three times by epicutaneous patching (at day 22, 36 and 50). Briefly, the foreskin of each mice were exposed epicutaneously with a small patch of gauze containing 50 μg of Der p2 allergen for three consecutive days. A month after the third sensitization, mice were challenged twice by aerosol with Der p2 (1 mg/10 ml PBS). Approximately 24 h after challenge, the bronchoalveolar lavage fluid (BALF) were obtained for differential cell count and cytokine analysis, and the lung for histological analysis. Spleen and mesenteric lymph nodes (MLN) were obtained for cell culture. Blood samples, taken at day 0 and every week, were centrifuged at 2000 g and sera were collected and stored at −20° C. for determination of antibody levels.

The kinetics of Der p 2-specific serum IgE levels in the Lc/Dp2 fed mice was lower than the NaHCO₃ and Lc/V fed mice, indicating that feeding with Lc/Dp2 alone can suppress IgE production even after airways challenge (FIG. 11). There was no significant difference in the Der p 2-specific serum IgG1 level for all the three groups. In the case of animal studies a concurrent down-regulation of Th-2 cytokines production by T-lymphocytes in Lc/Dp2 fed animals, e.g. mice, could also be monitored. For this purpose, spleen and mesenteric lymph node (MLN) cells were obtained for culture in the presence of Der p 2 or anti-CD3/CD28, respectively. As illustrated in FIG. 12, spleen T-cells from Lc/Dp2 fed mice with s.c. immunizations produced lower levels of Th-2 (IL-4, IL-5, IL-13) and pro-inflammatory cytokines (TNF-α), with a concurrent increase in TGF-β and a reduction in Der p 2-specific T-cell proliferation, when compared to the control groups. An analysis of MLN cells indicates that both L.c/V and Lc/Dp2 fed mice produced significantly lower levels of IL-13, a non-significant reduction in IL-4, IL-5 and IL-10 (with levels similar to Ac control mice) and elevated levels of TGF-β compared to NaHCO₃ group (cf. FIG. 13).

It is hypothesized that feeding with Lc/Dp2, primed for a mixture of Th-2 and Der p 2-specific Tr cells, the later were further expanded by two high-dose s.c. immunizations of Der p 2. These Der p2-specific Tr cells may be capable of exerting an inhibitory or tolerogenic effect on existing Th-2 cells via regulatory cytokines such as TGF-β1. The present inventors observed that Th-2 circulating IL-10 cytokine levels in sera of Lc/Dp2 fed mice obtained after airways challenge were significantly reduced compared to control groups. The cytokine profile of BALF from Lc/Dp2 fed mice showed a decreased in IL-13, IL-5, TNF-α, eotaxin (chemokine for eosinophils) and a significant reduction in TGF-β1 (FIG. 14). Concurrently, these mice also showed reduced a BALF cell count (FIG. 15A), with level similar to that of Ac group. In addition, only the Lc/Dp2 fed mice exhibited a significant reduction in neutrophils counts (cf. FIG. 15B). Except for a non-significant increase in lymphocytes count in BALF from Lc/Dp2 fed mice, the eosinophils count was low and similar for all groups.

Lung tissues of mice from NaHCO₃ or Lc/V groups showed a different degree (moderate to servere) of airway pathology and inflammatory infiltrates surrounding the bronchoalveolar spaces (FIG. 15C). In contrast thereto, lung sections from Lc/Dp2 fed mice showed substantial reduction in lung inflammation, having a profile similar to that of Ac mice (cf. FIG. 15C).

Mice primed by oral administration of Lc/Dp2 in combination with two s.c. boosting with Der p2 protein showed overall down-regulation of allergic responses, which is thus an effective prophylactic regimen. It is probable that this prime-boost regimen is capable of inducing a subset of antigen-specific Tr-cells that exert tolerance and/or down-regulate Th-2 modulators at both B and T-cell levels as well as in the airways, thereby efficiently blocking the pathogenesis of allergic asthma and air-way remodeling in this mouse model. It has been reported that murine Tr cells such as the CD25⁺CD4⁺, Tr1 and Th3 play a critical role in the down-regulation of asthma and allergy. The mechanism of induction and the role of Tr cells and regulatory cytokines involved will be further illustrated.

### Example 13

### Therapeutic Regimen in an Asthma Mouse Model

The present example illustrates the efficacy of orally administered heat-killed recombinant Lc/Dp2 or wildtype Lc/V in a therapeutic regimen, in combination with an immunotherapy by subcutaneous immunization of Der p 2 protein on a Der p 2-induced asthma mouse model (FIG. 16). The control group used for the depicted data consisted of mice fed with NaHCO₃ buffer. In this regimen, mice were pre-sensitized by epicutaneous patching and subsequently fed with either heat-killed Lc/V or Lc/Dp2 for five consecutive weeks. All mice received two subcutaneous immunizations at the last two weeks of feeding, thus mimicking the subcutaneous immunization employed in immunotherapy.

Briefly, C57BL/6 mice were pre-sensitized by epicutaneous patching (at day 0, 14 and 28) with Der p 2 (50 μg/mouse). Briefly, C57BL/6 mice were pre-sensitized by epicutaneous patching (at day 0, 14 and 28) with Der p2 (50 μg/mouse), as described in Example 12. At day 33, the profile of Der p 2-specific sera IgE and IgG1 were determined by ELISA and based on IgE levels, the mice were subsequently divided into three groups of six mice each. Mice were fed orally with either NaHCO₃ buffer, heat-killed L. casei Shirota/pLP500 (Lc/V, wildtype control) or L. casei Shirota/Derp2 (Lc/Dp2); oral feeding was carried out using autoclaved gavage needles (Popper & sons, inc., NY). An aliquote of frozen heat-killed Lc/V or Lc/Dp2 described previously were thawed to room temperature and each mouse received one dose of 100 μl containing 109 heat-killed Lc/V or Lc/Dp2 or 100 μl of NaHCO₃ buffer per day, for three consecutive days in a week, for 5 weeks, starting at day 35. At day 55 and 62, mice were immunized with two subcutaneous injections of Der p 2 (50 μg/mouse) and subsequently challenged twice by aerosol with Der p 2 allergen (1 mg/10 ml PBS mice were subsequently challenged twice by aerosol with Der p 2 (1 mg/10 ml PBS). Approximately 24 h after the last challenge, the BALF were obtained for differential cell count and cytokine analysis, and lung for histological studies.

As FIG. 17 shows, mice in all three groups show a decrease in Der p 2-specific IgE one week after the start of feeding. Although the IgE level was elevated after the first subcutaneous immunization with high dose Der p 2 allergen at day 62, the IgE level dropped significantly for Lc/V and Lc/Dp2 fed mice compared to control mice before and after two consecutive aerosol challenges (at day 69 and 77) (cf. FIG. 17B). Contrary, the Der p 2-specific sera IgG1 was significantly elevated for these two groups compared to control mice at day 62 and 69, after subcutaneous immunizations and remained unchanged even after airways challenged (FIG. 17C and FIG. 17D).

To determine whether there is down-regulation of Th-2 cytokines production by T-lymphocytes in Lc/V and Lc/Dp2 fed mice compared to control group, spleen T-cells and mesenteric lymph nodes cells were obtained for culture in the presence of Der p 2 or anti-CD3/CD28, respectively. Der p 2-specific spleen T-cells from both Lc/Dp2 and Lc/V fed mice showed significant decrease in Th-2 cytokines (IL-5, IL-13, IL-10) and a non-significant decrease in IL-4 and TNF-α compared to the NaHCO₃ group (FIG. 18). Interestingly, there was non-significant decrease in TGF-β1 production for both the groups. Similar to spleen T-cells, the mesenteric lymph nodes (MLNs) cells from both the Lc/Dp2 and Lc/V groups exhibited non-significant decrease in Th-2 and pro-inflammatory cytokines (IL-5, IL-4, IL-13, IL-10 and TNF-α) production (FIG. 19). Contrary, the TGF-β1 production in MLNs cells was elevated for both the LcDp2 and Lc/V group compared to NaHCO₃ group (FIG. 19F).

The cytokine profile of BALF from both Lc/V and Lc/Dp2 groups compared to the NaHCO₃ group exhibited a non-significant decrease in both Th-2 and pro-inflammatory cytokines (IL-5, IL-13, IL-4, IFN-γ, TNF-α, TGF-β) and eotaxin, a chemokine for eosinophils recruitment. Both groups have levels similar to that of Ac control group (cf. FIG. 20). Both the Lc/V and the Lc/Dp2 group showed a similar number of total infiltrating cells compared to the aerosol control group (Ac), being non-significantly lower than observed in the NaHCO₃ group (cf. FIG. 21). Differential cell analyses indicated a reduction in neutrophils and eosinophils for both of these groups, while lymphocytes and macrophages counts were unaffected. Only the Lc/Dp2 group exhibit slight reduction in monocytes count in the broncholalveolar lavage fluid (BALF) compared to the other groups.

Lung tissues from two representative mice out of six in each group were shown (cf. FIG. 21). Haematoxylin & Eosin (H&E) staining of lung sections from two aerosol control mice (G and H) showed a background of minimal airway inflammation in lung parenchyma with minimal inflammatory infiltrates in the bronchiolar spaces. However lung tissues of mice from NaHCO₃ group exhibited different degree (moderate to severe) of airway inflammatory infiltrating cells surrounding the airways and bronchiolar spaces (A and B). Comparatively, both the Lc/V (c-d) and Lc/Dp2 (E-F) fed mice showed a greater reduction in inflammatory infiltrates, having profile similar to that of Ac mice.

### Example 14

### Effect of Oral Administration of Live L. Casei Shirota/Dp2 on Mice Presensitized with Der P2

While it is understood that the forgoing examples may likewise be performed using live recombinant lactobacilli instead of heat-inactivated lactobacilli, the present example illustrates the use of live recombinant L. casei Shirota expressing Der p 2.

C57/B 6 mice were bled weekly to determine their Der p 2-specific IgE and IgG1 titers. All assays were measured by ELISA (cf. FIG. 32). Mice were patched epicutanously with 50 μg of recombinant yeast-derived Der p 2 allergen in 100 μl of PBS for 3 days. They were patched for three times at d1, d14 and d28. After resting for 14 days, only IgE responders were divided equally into two groups (Lb vs buffer) for treatment studies.

Approximately 10⁹ cells of live recombinant Der p 2 (Lc/Dr p2) in 501 of bicarbonate buffer were fed to each of the six mice in the Lb group. Cells were washed twice with bicarbonate buffer before feeding. Only sodium bicarbonate buffer was given to control mice. Feeding was carried out daily and lasted for a total of 4 weeks.

Both groups were challenged (aerosol) 10 days after last feeding. Mice from the same group were placed in a chamber. 1 mg of recombinant yeast-derived Der p 2 in 10 ml of PBS was nebulized into the chamber and inhaled by the mice for 30 minutes

C57BL/6 mice were sensitized by epicutaneous patching with Der p 2 allergen (50 μg/mouse) at day 0, 14 and 28. The Der p 2-specific IgE levels were assayed at day 42 and responders were divided into two groups (n=6) according to their respective IgE titers. Oral feeding was carried out daily for 1 month, one group were fed with NaHCO₃ control and the other with 1 dose (1×10⁹ cfu)/mouse of recombinant L. casei Shirota/Dp2. At day 80, the mice were aerosol challenged once before sacrificing at day 82 and spleen obtained for T-cell culture (cf. FIG. 22).

The immune response of the two groups of mice was analysed based on systemic IgG1 and IgE production and the cytokine profiles of spleen T-cells. Pre-sensitized mice when fed with L. casei Shirota/Dp2 can efficiently lowered the Der p 2-specific serum IgE and IgG1 levels. As shown in FIG. 23, the L. casei Shirota/Dp2 fed mice showed a 41% attenuation of the Der p2 specific serum IgE approximately 7 days post feeding compared to NaHCO₃ control group which showed only 27% attenuation of IgE (cf. FIG. 23A). In addition, the Der p 2-specific serum IgG1 was significantly attenuated the L. casei Shirota/Dp2 fed mice compared to the NaHCO₃ control group (cf. FIG. 23B).

However the spleen T-cells profile of L. casei Shirota/Dp2 fed group was similar to the NaHCO₃ control group in terms of TH1 and TH2 cytokine productions (cf. FIG. 24). In addition, mice fed with L. casei Shirota/Dp2 showed an increase in T-regulatory cytokines (IL-10 and TGF-β) production (cf. FIG. 23.C). In this therapeutic model, the profile of airway inflammation in these mice was not examined.

### Example 15

### Subcutaneous Priming of High Dose Der P 2 Led to Attenuation of IgE and Tr-Associated Cytokines Production in Mice Challenged with Der P 2

This example illustrates the measurement of the dosage effect of Der p 2 allergen on the regulation of antibody production without the application of adjuvant in mice.

Groups of 8 female mice (6 to 8 weeks old) were administered three times at four days intervals by s.c. injection with low dose (LD) [10 μg/mouse] or high dose (HD)[50 μg/mouse] of yeast recombinant Der p 2 protein (cf. FIG. 1). A boost with LD of Der p 2 was given to the immunized mice on day 28. In the aerosol challenge, mice were kept unrestrained in dessicator chamber and given continuous flow of aerosol Der p 2 generated by an ultrasonic nebulizer (model UltraNEB 99, DeVilbiss Health Care, Somerset, Pa.). Four dosages of 0.1 mg/ml of Der p 2 in PBS were given for 30 mins at two days interval. Control mice were subjected to aerosol Der p 2 inhalation alone. Sera were collected and analyzed for Der p 2-specific antibodies (cf. FIG. 28).

### (a) ELISA for Der p 2-Specific Antibodies

The level of Der p 2-specific IgG1, IgE and IgG2a antibodies were measured by ELISA. ELISA plates (Costar, Corning, N.Y., USA) were incubated with recombinant Der p 2 at 5 μg/ml in coating buffer (0.1 M NaHCO₃, pH 8.3) at 4° C. overnight. All reagents were used in volumes of 50 μl/well unless stated otherwise. After incubation, plates were washed three times with PBS/0.05% Tween 20 and blocked with 100 μl of blocking buffer (1% BSA in PBS/0.05% Tween 20) for 1 hr at room temperature. The plates were incubated overnight at 4° C. with serially diluted sera. Plates were washed and incubated with biotinylated monoclonal rat anti-mouse IgG1 (G1-1.5), IgG2a (R35-92) or IgE (R19-15) (Serotec, Oxford, England) at 250 μg/ml for 1 hr at room temperature. Plates were washed and incubated with alkaline-phosphatase-conjugated ExtrAvidin (Sigma Chemical Co, St Louis, Mo.) (1:2000 dilution). Plates were washed six times and developed using Sigma Fast pNitrophenyl phosphatase substrate. After 1 hr incubation, the absorption was measured at 405 nm using ELISA plate reader (Tecan G.m.b.H, Austria.). A similar protocol was engaged to generate a standard curve except for the following changes. Anti-mouse Igκ light chain Ab (187.1) (Pharmingen, San Diego, Calif.) was coated in duplicate wells, and mouse recombinant IgG1 (107.3), IgG2a (G155-178) or IgE (C38.2) (Phanningen) were serially diluted in 2-folds starting from 50 ng/ml. Antibody titers were compared to the standards. ELISA unit (EU) was defined as the OD₄₀₅ₙₘ reading corresponding to the reading of 1 ng/ml of detected antibody in a sandwich ELISA with anti-mouse Igκ as the capture antibody in the same plate.

Repetitive s.c. injections with low dose (LD) Der p 2 allergen were found to stimulate high IgE production (4.0±0.7 EU) that drastically increased (>4-fold) one week after boosting (FIG. 26A, white squares). IgG1 production remained persistently low (3280±550 EU) until day 64, where a rise occurred most likely due to exposure to aerosolize Der p 2 (FIG. 26B, white squares). IgG2a levels remained low (104±67 EU, FIG. 26C, white squares). On the contrary, mice primed with high dose (HD) of Der p 2 allergen had a low basal IgE level (1.30±0.33 EU, FIG. 26A, black squares). This group of mice showed persistently higher IgG1 production (8130±1000 EU, (FIG. 26B, black squares) that rose greatly after boosting (44800±4250 EU). Their IgG2a levels were also higher than that in mice primed with LD, particularly after boosting or inhalation challenged (340±260 EU, FIG. 26C, black squares).

### (b) Splenic T-cell Culture

Treated mice were sacrificed by cervical dislocation on day 64. Spleens were excised from the mice and depleted of erythrocytes by using RBC lysis buffer (0.53% ammonium chloride). Cells were cultured with 10 μg/ml of Der p 2 allergen at 2×10⁶ cells/ml in RPMI-1640 medium supplemented with 10% of heat-inactivated fetal calf serum (FBS) [Hyclone Laboratories, Logan, Utah], 2 mM L-glutamine (Hyclone Laboratories), 1 mM sodium pyruvate (Gibco BRL), 5.5×10⁻² mM of β-mercaptoethanol (Life Technology, Grand Island, N.Y.), antibiotics (100 U/ml Penicillin and 100 μg/ml Streptomycin) (Hyclone Laboratories) in 5% CO₂ incubator at 37° C. Mouse recombinant IL-2 (R & D systems, Minneapolis, Minn., USA) was 10 U/ml was added on day 3, 5, 7. Viable cells were recovered by using ficoll gradient centrifugation (Ficoll pague plus, Pharmacia) on day 10.

### (c) CD4⁺ T-cells Enrichment and Stimulation

CD4⁺ T-Cells were enriched by AutoMacs (Miltenyi Biotec, Bergisch Gladbach, Germany) according to the manufacturers' instructions, using anti-mouse CD4 biotin-conjugated antibody (GK1.5) and streptavidin-conjugated microbeads (Miltenyi Biotec). Before undergoing the enrichment protocol, spleen cells of naïve mice were blocked with 5% FCS/PBS and anti-mouse Fcγ III/II receptor (CD16/CD32) [2,4G2], and depleted of natural killer cells and dendritic cells by using anti-Pan NK biotin-conjugated Ab (DX5), anti-CD11c microbeads and streptavidin-conjugated microbeads. The cell purity was determined by flow cytometry analysis using FACScan flow cytometry and CellQuest software (Becton Dickinson). At least 95% of the cells were CD4⁺ T cells for all groups. Purified CD4⁺ T cells were cultured in 96-well plate at 3×105 cells/200 μl and stimulated for 24 hr with anti-mouse CD36 Ab (5 μg/ml, 145-2C11) and anti-mouse CD28 Ab (2 μg/ml, 37.51)

### (d) cDNA Generation, Amplification and Real-Time PCR

Cells were lysed and total RNA was isolated using the RNeasy Min Kit (Qiagen Inc, CA) according to the manufacturer instructions. cDNA was generated from 2 μg of total RNA using 1 μg of 15mer poly d(T) oligonucleotides and 20 units of Moloney-Murine Leukaemia Virus (M-MVL) Reverse Transcriptase (Promega, Madison, USA) as recommended.

HPRT was normalized in each sample so as to standardize the amount of cDNA sample used in each PCR. The cDNA was added to a reaction mixture containing 10× PCR buffer, 10 μmol of cytokine primers, 0.5 mM dNTP and 2.5 units of Taq DNA polymerase. Each sample of 25 μl final volume was incubated in a DNA thermal cycler (Perkin Elmer Gene Amp PCR system 9700, PE applied biosystems, USA) for a total of 30 cycles. Each cycle consists of 30 sec at 94° C., 30 sec at 58° C. and 1 min at 72° C. 1 min. A starting of 5 mins incubation at 94° C. and a final extension of 10 mins at 72° C. was included in each reaction.

Real-time PCR using SYBR Green technology in LightCycler was carried out amplifying cDNA samples, negative control (water) and a series of diluted standard. Naïve mice sample was used as standard to create the fit coefficients file for each cytokine. Reactions were performed using Fast start DNA Master SYBR Green I (Roche Diagnostics, Switzerland) in accordance to the manufacturers' instructions. The amplification program was: denaturation 10 min at 95° C., quantitation 40 cycles of 5 sec at 95° C., 5 sec at 58° C. and 12 sec at 72° C., melting 15 sec at 58° C., cooling 30 sec at 40° C. The cytokine relative ratios were calculated with efficiency correction based on a non-linear regression fit performed automatically by the Relative Quantification Software (Roche Molecular Biochemicals, Germany).

The following oligonucleotides were used for PCR analysis:

(SEQ ID NO: 7)

HPRT: Sense 5′ GTTGGATACAGGCCAGACTTTGTTG 3′

(SEQ ID NO: 8)

Anti-sense 5′ GAGGGTAGGCTGGCCTATGGG 3′;

(SEQ ID NO: 9)

IFN-γ: Sense 5′ CATTGAAAGCCTAGAAAAGTCTG 3′

(SEQ ID NO: 10)

Anti-sense 5′ CTCATGAATGCATCCTTTTTCG 3′;

(SEQ ID NO: 11)

IL-4 Sense 5′ CATCGGCATTTTGAACGAGGTCA 3′;

(SEQ ID NO: 12)

Anti-sense 5′ CTTATCGATGAATCCAGGCATCG 3′;

(SEQ ID NO: 13)

IL-5 Sense 5′ GAAAGAGACCTTGACACAGCTG 3′;

(SEQ ID NO: 14)

Anti-sense 5′ GAACTCTTGCAGGTAATCCAGG 3′;

(SEQ ID NO: 15)

IL-9: Sense 5′ ATGTTGGTGACATACATCCTTGC 3′;

(SEQ ID NO: 16)

Anti-sense 5′ CGGCTTTTCTGCCTTTGCATCTC;

(SEQ ID NO: 17)

IL-10: Sense 5′ CCAGTTTTACCTGGTAGAAGTGATG 3′;

(SEQ ID NO: 18)

Anti-sense 5′ TGTCTAGGGTCCTGGAGTCCAGCAGACT 3′;

(SEQ ID NO: 19)

IL-12 Sense 5′ ATGGCCATGTGGGAGCTGGAG 3′;

(SEQ ID NO: 20)

Anti-sense 5′ TTTGGTGCTTCACACTTCAGG 3′;

(SEQ ID NO: 21)

IL-13: Sense 5′ ATGGCCATGTGGGAGCTGGAG 3′;

(SEQ ID NO: 22)

Anti-sense 5′ TTTGGTGCTTCACACTTCAGG 3′:

### (e) Immunization Regimen for Study of CD4⁺CD25⁺ Regulatory T-Cells

Mice were primed by subcutaneous injection (s.c.) with low dose (LD) [10 μg/mouse] or high dose (HD) [50 μg/mouse] of yeast recombinant Der p 2 protein (rDer p 2) on day 0, 4 and 8. A boost with LD of Der p 2 was given on day 28 and Der p2-specific immune response was assayed by ELISA. In another study, rDer p 2-primed mice were sacrificed on day 21. Lymph nodes and spleens were obtained for cytokine profiling of T-cell cultures. Controls mice were s.c. injected with 100 μl of PBS. The rDer p 2-epicutaneous patched mice were generated as previously described (Wang L F et al 1996). Briefly, 50 μg of rDer p 2 in 100 μl of PBS was first applied to 1 cm² gauze on the patches, which was then applied to the shaved skin and secured with an elastic bandage. The patching was performed on day 0 and 14. Each patch was applied for 4 days and removed. Mice were sacrificed on day 21 and antigen-specific TH2 cells were established.

Control mice receiving only aerosol challenge with Der p 2 gave high expression levels of Ag-specific TH2 cytokines specifically IL-5, IL-10 and IL-13. However the HD primed mice showed significant suppression of IL-13, IL-5 and IL-10 expression, when compared with mice primed with LD or control group. In addition, the LD primed mice had exclusively high expression of IL-9 expression compared to HD primed mice and control. Thus the initial HD priming ameliorated the effects of inhaling Der p 2 by suppressing the expression of TH2-associated cytokines.

### (f) Enrichment of CD4⁺CD25⁺ Regulatory T-Cells

Splenic regulatory T-cells are enriched by using CD4⁺CD25⁺ Regulatory T-Cell Isolation Kit (Miltenyi Biotec, Bergisch Gladbach, Germany) and sorted using AutoMacs (Becton Dickinson) according to the manufacturers' instructions. A portion of the isolated cells were incubated with FITC-conjugated anti-mouse CD4 antibodies, Per-CP-conjugated anti-mouse CD3ε antibodies, and PE-conjugated anti-mouse CD25 for purity check. The cell purity was determined by flow cytometry analysis using FACScan flow cytometry and CellQuest software (Becton Dickinson), and at least 95% of isolated cells were CD4⁺CD25⁺ T-cells

### (g) Splenocyte Cultures and Antigen Presenting Cells (APCs) Preparation

Splenocytes were cultured in complete RPMI-1640 medium supplemented with 10% heat-inactivated bovine calf serum (StemCell Technologies), 1 mM sodium pyruvate (Hyclone Laboratories), 2 mM L-glutamine, antibiotics (100 U/ml penicillin and 100 μg/ml streptomycin), 5.5×10⁻² mM 2-β mercaptoethanol (Life Technology), and maintained at 37° C. in 5% CO₂ incubator. Splenocytes were cultured in 96-well (4×10⁵ cells/well) with 10 μg/ml of Der p 2 protein for 3-5 days and supernatant harvested were frozen at −20° C. Antigen-specific TH2 cells were established from epicutaneous patched mice by culturing the splenocytes in 6-well plates (2×10⁷ cells/well) with rDer p2. The cells were supplemented with fresh medium containing 10 U/ml of recombination mouse IL-2 (rIL-2) (R & D systems) on day 3, 5 and 7. At day 10, TH2 cells were harvested and purified by Ficoll-Pague plus (Amersham Biosciences) centrifugation. APCs were derived from the mitomycin C-treated splenocytes of naive mice. Briefly, mitomycin C (Roche Diagnostic GmbH, Mannheim, Germany) was added to the cells at a final concentration of 50 μg/ml and incubated in the dark at 37° C. for 20 min. The cells were washed 3 times with 30 mls of 1× HBSS and suspended in RPMI-1640 medium.

### (h) Cytokine Profiling and Cell Proliferation Assay

Antigen-specific TH2 cells and CD4^{+CD}25⁺ T-cells were cultured at 1×10⁵ cells/well with or without 10 μg/ml of rDer p 2. APCs were used at 3×10⁵ cells/well. Supernatants were harvested at day 3 and assayed for IL-4, IL-5 and IL-13 production. In the proliferation study, cells were incubated for 5 days and pulsed with 1 μCi of [³H]-thymidine (NEN Life Science, Boston, Mass.) at the last 18 hr. Cells were harvested to a glass fiber filter (Skatron instruments AS, Lier, Norway). After adding the scintillation fluid (Amersham Biosciences Corp) and proliferation was measured by the liquid scintillation counter (Beckman Coulter, Inc. Fullerton, Calif.). The proliferation index is expressed as the ratio of TH2 cells alone.

### (i) Cytokine ELISA

Purified antibodies to mouse IFN-γ (RA-6A2), IL-4 (BVD4-1D11), IL-5 (TRFK5), IL-9 (D8402E8), IL-10 (JES052A5) (R & D systems, Minneapolis, Minn., USA), IL-13 (38213) (R & D systems) and TGF-β (A75-2.1) antibodies were coated on 96-well plate at 2 μg/ml. Biotinylated polyclonal antibodies to mouse IFN-γ (XMG1.2), IL-4 (BVD6-24G2), IL-5 (TRFK4), IL-9 (D9302C12), IL-10 (R & D systems), IL-13 (R & D systems) and TGF-β (A75-3.1) antibodies were used as recommended. Recombinant mouse cytokines were used as standards in the ELISA assay. All antibodies and recombinant cytokines were from BD Biosciences PharMingen (San Diego, Calif.) unless stated otherwise.

FIG. 27 depicts the quantification of cytokine mRNA expression in Der p 2-primed mice that were challenged with aerosolized Der p 2. IL-12 was not detected and there was no difference in the expression levels of IL-4 and IFN-γ between the groups of mice. There were, however, differences in their expression of other TH2 cytokines especially effector cytokines.

The expression levels of these cytokines were further evaluated using real-time PCR (cf. Table I). The melting curve analysis of each amplified kit showed distinctive, sharp peak that was not observed in water control (data not shown). Table I illustrates the calibrator (untreated)-normalized amplified cytokine kit/HPRT ratio. There was no distinctive difference in IL-4 expression in all groups of mice. Control mice subjected to aerosol Der p 2 inhalation gave high expression levels of Ag-specific TH2 cytokines specifically IL-5, IL-10 and IL-13. HD primed mice showed significant suppression of IL-13 expression (at least 100-fold lower), IL-5 and IL-10 expression (˜7-fold less), when compared with mice primed with LD or control group. This implies that the initial HD priming ameliorated the effects of inhaling Der p 2 by suppressing the expression of TH2-associated cytokines. LD primed mice had exclusively high expression of IL-9 expression (>100-fold) compared to HD primed mice and control. Accordingly, TH2 cytokines are associated with the allergen dosage immunisation.

FIGS. 27 and 28 show cytokine profiles of lymph nodes and spleen from mice primed with high or low dose rDer p 2 protein. Mice were primed with low dose (LD, 10 μg) or high dose (HD, 50 μg) of rDer p 2 protein, or PBS on day 0, 4, 8 and sacrifice on day 10 (supra). Lymph nodes and spleens were harvested and cultured for 3 to 5 days in the presence of rDer p 2 protein. Both lymph nodes and spleen and from mice primed with low dose showed increase in TH2 cytokines (IL-4, IL-13 and IL-9) and decrease in TH1 cytokine (IFN-γ) production compared to high dose primed mice. On the other hand, the high dose primed mice exhibited higher TGF-β production in both lymph nodes and spleen.

FIG. 30 depicts the suppressive effect of CD4⁺CD25⁺ regulatory T-cells from mice primed with high dose rDer p 2 protein. Splenic CD4⁺CD25⁺ T cells of mice primed with LD or HD rDer p 2 protein were cocultured with antigen-specific TH2 cells in the presence of rDer p 2 protein for 5 days and assayed for both proliferation and cytokine response. As shown in FIG. 30 (A-D), splenic CD4⁺CD25⁺ T cells from mice primed with HD rDer p 2 protein were able to exert suppression both on the proliferation of TH2 cells and production of TH2 cytokines, IL-4, IL-5 and IL-13. Splenic CD4⁺CD25⁺ T cells from mice primed with LD rDer p 2 protein were unable to exert similar suppressive effects.

### Example 16

### Studies of the Immune Responses Primed by Recombinant L. Casei Shirota/Blo t 5 in Mice

Mice were fed with 1×10⁹ cfu/mouse each day for four consecutive days per week (FIG. 31) Total feeding each mouse received were 20 doses of 1×10⁹ cfu. Mice were bled weekly up to 7 weeks and Blo t 5-specific serum immunoglobulins were assayed by ELISA. There were significant levels of Blo t 5-specific IgG1 detected but there were no significant levels of IgE and IgG2a were detected. (FIG. 32). Mice were sacrificed on day 198 and T-cells from Peyer's patches and spleen were obtained for cytokine analysis. Significant levels of regulatory cytokine TGF-β were detected in T cells from Peyer's patches of the L casei Shirota/Blo t 5 fed mice only, indicating that the live recombinant L casei Shirota I Blo t 5 actively induced production of T regulatory cytokines by T cells in Peyer's patches (cf. FIG. 33).

The listing or discussion of a previously published document in this specification should not necessarily be taken as an acknowledgement that the document is part of the state of the art or is common general knowledge. All documents listed are hereby incorporated herein by reference.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by a preferred embodiment, modification and variation of the invention herein disclosed may be resorted to by those skilled in the art, and that such modifications and variations are considered to be within the scope of this invention.

The invention has been described broadly and generic herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, the skilled artisan will recognize that the invention is also thereby described in terms of any individual member or subgroup of members of the Markush group.

TABLE 1

| | Cytokine ratio* | | | | |
|---|---|---|---|---|---|
| Treatment | IL-4 | IL-5 | IL-9 | IL-10 | IL-13 |
| Low dose | 1.1 | 10.0 | 158.3 | 40.4 | 156.3 |
| High dose | 1.6 | 3.9 | 5.0 | 20.5 | 4.8 |
| Control | 1.0 | 34.4 | 1.6 | 51.4 | 103.2 |
| Naive | 1.0 | 1.0 | 1.0 | 1.0 | 1.0 |

1-85. (canceled)

86. A recombinant lactobacillus comprising a heterologous nucleic acid sequence encoding at least an immunogenic fragment of the mite allergens Der p 2 or Blo t 5, or an immunogenic homolog thereof, wherein said heterologous nucleic acid sequence encoding an immunogenic homolog of Der p 2 or an immunogenic fragment thereof has a nucleic acid sequence of at least 70% identity to the nucleic acid sequence of SEQ ID NO: 1.

87. The recombinant lactobacillus of claim 86, wherein said lactobacillus is selected from the group consisting of Lactobacillus casei, Lactobacillus acidophilus, Lactobacillus fermentum, Lactobacillus gasseri, Lactobacillus pentosus, Lactobacillus plantarum, Lactobacillus sporogenes, Lactobacillus brevis, Lactobacillus delbrueckii, Lactobacillus salivarius, Lactobacillus hilgardii, Lactobacillus lactis, Lactobacillus rhamnosus, Lactobacillus johnsonii, Lactobacillus leishmanis, Lactobacillus jensenii, Lactobacillus reuteri, Lactobacillus sakei, Lactobacillus cellobiosus, Lactobacillus crispatus, Lactobacillus curvatus, Lactobacillus caucasicus, and Lactobacillus helveticus.

88. The recombinant lactobacillus of claim 87, wherein said lactobacillus is Lactobacillus rhamnosus GG or Lactobacillus casei Shirota.

89. The recombinant lactobacillus of claim 86, wherein said immunogenic homolog encoded by said heterologous nucleic acid sequence has at least 80% sequence identity to the amino acid sequence of a respective at least immunogenic fragment of said mite allergen.

90. The recombinant lactobacillus of claim 89, wherein said mite allergen is Der p 2 or Blo t 5 and said lactobacillus is Lactobacillus casei Shirota.

91. The recombinant lactobacillus of claim 86, wherein said mite allergen is Der p 2 and wherein said mite allergen is encoded by the sequence of SEQ ID NO: 1.

92. The recombinant lactobacillus of claim 86, wherein said mite allergen is Blo t 5 and wherein said at least immunogenic fragment of said mite allergen is encoded by the sequence of SEQ ID NO: 2.

93. The recombinant lactobacillus of claim 86, wherein said mite allergen is Blo t 5 and wherein said heterologous nucleic acid sequence encodes an immunogenic homolog of Blo t 5, or an immunogenic fragment thereof, the immunogenic homolog having a nucleic acid sequence of at least 70% identity to the nucleic acid sequence of SEQ ID NO: 2.

94. The recombinant lactobacillus of claim 86, wherein said sequence encoding at least an immunogenic fragment of said mite allergen or an immunogenic homolog thereof is comprised in a heterologous nucleic acid molecule.

95. The recombinant lactobacillus of claim 94, wherein said heterologous nucleic acid molecule is an expression vector.

96. The recombinant lactobacillus of claim 95, wherein said expression vector is selected from the group consisting of pLP400, pLP500, pSIP308 and pSIP412.

97. A pharmaceutical composition comprising a recombinant lactobacillus according to claim 86.

98. The pharmaceutical composition of claim 97, further comprising a pharmaceutically acceptable carrier or diluent.

99. The pharmaceutical composition of claim 97, further comprising at least one of a corticosteroid, an antihistamine, a leukotriene modifying agent, a mast cell stabilizer, a decongestant and a β2-adrenoceptor agonist.

100. The pharmaceutical composition of claim 97, further comprising at least an immunogenic fragment of an allergen, or an immunogenic homolog thereof.

101. The pharmaceutical composition of claim 100, wherein said allergen is a mite allergen.

102. The pharmaceutical composition of claim 101, wherein the mite allergen is a dust mite allergen.

103. The pharmaceutical composition of claim 101, wherein said mite allergen is selected from the group consisting of Der p 1, proper p 1, Der p 2, Der p 3, Der p 4, Der p 5, Der p 7, Der p 8, Der p 9, Der p 10, Der p 11, Der p 14, Der p 15, Der p 18, Der f 1, Der f 2, Der f 3, Der f 4, Der f 5, Der f 6, Der f 7, Der f 10, Der f 11, Der f 15, Der f 16, Der f 18, Der m 1, Eur m 1, Eur m 2, Her f 2, Blo t 1, Blo t 3, Blo t 5, Blo t 12, Fel d 1, Mag 1, Mag 3, Tyr p 2, Lep d 1, Lep d2, Lep d5, Lep d7, Lep d 10, and Lep d 13.

104. The pharmaceutical composition of claim 101, wherein said mite allergen comprises at least one common epitope with the at least immunogenic fragment of a mite allergen, or immunogenic homolog thereof, expressed by said recombinant lactobacillus.

105. The pharmaceutical composition of claim 104, wherein the at least immunogenic fragment of a mite allergen, or immunogenic homolog thereof, is the at least immunogenic fragment of a mite allergen, or immunogenic homolog thereof, expressed by said recombinant lactobacillus.

106. A pharmaceutical kit comprising in two separate parts
(a) a pharmaceutical composition as defined in claim 97, and
(b) a pharmaceutical composition comprising at least an immunogenic fragment of an allergen, or an immunogenic homolog thereof.

107. A method of modulating the immune response to an allergen in a mammal, said method comprising administering a pharmaceutical composition as defined in claim 97.

108. The method of claim 107, wherein said mammal is a human.

109. The method of claim 107, wherein said allergen is a mite allergen.

110. The method of claim 109, wherein said mite allergen is a dust mite allergen.

111. The method of claim 107 in the treatment or prophylaxis of an allergic disease.

112. The method of claim 111, wherein said allergic disease is a mite allergy.

113. The method of claim 112, wherein said allergic disease is selected from the group consisting of asthma, rhinitis, atopic dermatitis, and urticaria.

114. The method of claim 107, wherein the pharmaceutical composition is administered orally or sublingually.

115. The method of claim 107, comprising repeatedly administering said pharmaceutical composition that comprises recombinant lactobacillus.

116. The method of claim 107, wherein said pharmaceutical composition further comprises at least an immunogenic fragment of an allergen, or an immunogenic homolog thereof.

117. The method of claim 107, further comprising administering a pharmaceutical composition that comprises at least an immunogenic fragment of an allergen, or an immunogenic homolog thereof.

118. The method of claim 117, wherein said pharmaceutical composition comprising a recombinant lactobacillus and said pharmaceutical composition comprising said at least immunogenic fragment of an allergen, or immunogenic homolog thereof, are comprised in a pharmaceutical kit.

119. The method of claim 117, wherein the allergen, of which at least an immunogenic fragment, or an immunogenic homolog thereof, is comprised in said pharmaceutical composition, is a mite allergen.

120. The method of claim 117, wherein said at least immunogenic fragment of an allergen, or immunogenic homolog thereof, comprises at least one common epitope with the at least immunogenic fragment of an allergen, or an immunogenic homolog thereof, expressed by said recombinant lactobacillus.

121. The method of claim 120, wherein said at least immunogenic fragment of an allergen, or immunogenic homolog thereof, is the at least immunogenic fragment of an allergen, or immunogenic homolog thereof, expressed by said recombinant lactobacillus.

122. The method of claim 117, wherein said at least immunogenic fragment of an allergen, or immunogenic homolog thereof, is obtained by any one of enrichment, purification and isolation from a recombinant organism.

123. The method of claim 117, wherein said pharmaceutical composition comprising said at least immunogenic fragment of an allergen, or immunogenic homolog thereof, is administered in a manner selected from the group consisting of sublingually, subcutaneously, intradermally, transdermally, epicutaneously and any combination thereof.

124. The method of claim 117, wherein said pharmaceutical composition comprising at least an immunogenic fragment of an allergen, or an immunogenic homolog thereof is administered subcutaneously, and
wherein the pharmaceutical composition according to claim 97 is administered orally.

125. The method of claim 117, wherein said pharmaceutical composition comprising said recombinant lactobacillus and said pharmaceutical composition comprising said at least immunogenic fragment of an allergen, or immunogenic homolog thereof are administered sequentially.

126. The method of claim 125, comprising:
(a) providing a pharmaceutical composition according to claim 97,
(b) administering the pharmaceutical composition,
(c) providing a pharmaceutical composition comprising at least an immunogenic fragment of an allergen, or immunogenic homolog thereof, and
(d) administering the pharmaceutical composition comprising at least an immunogenic fragment of an allergen, or immunogenic homolog thereof.

127. The method of claim 117, wherein said pharmaceutical composition comprising at least an immunogenic fragment of an allergen is administered first, and the pharmaceutical composition according to claim 97 is administered thereafter.

128. The method of claim 117, wherein said method is immunotherapy.

129. The method of claim 117, wherein said pharmaceutical composition comprising said at least immunogenic fragment of an allergen, or immunogenic homolog thereof, is administered repeatedly.
